(19) United States
(12) Patent Application Publication
(10) Pub. No.: US 2017/0239468 A1
(43) Pub. Date: Aug. 24, 2017

(54) ELECTROTRANSPORT DRUG DELIVERY DEVICES AND METHODS OF OPERATION

(71) Applicants: John LEMKE, Pleasanton, CA (US); Scot SATRE, Brentwood, CA (US); Corinna X. CHEN, Oakland, CA (US); Brian W. READ, Brier, WA (US); Zita S. NETZEL, Los Altos, CA (US); David SEWARD, Seattle, WA (US); Bradley E. WHITE, Lebanon, OH (US); Paul HAYTER, Mountain View, CA (US); Jason E. DOUGHERTY, Seattle, WA (US)

(72) Inventors: John LEMKE, Pleasanton, CA (US); Scot SATRE, Brentwood, CA (US); Corinna X. CHEN, Oakland, CA (US); Brian W. READ, Brier, WA (US); Zita S. NETZEL, Los Altos, CA (US); David SEWARD, Seattle, WA (US); Bradley E. WHITE, Lebanon, OH (US); Paul HAYTER, Mountain View, CA (US); Jason E. DOUGHERTY, Seattle, WA (US)

(21) Appl. No.: 15/589,754

(22) Filed: May 8, 2017

Related U.S. Application Data
(63) Continuation-in-part of application No. 14/296,085, filed on Jun. 4, 2014.
Application No. 14/296,085 is a continuation-in-part of application No. 13/249,975, filed on Sep. 30, 2011, now Pat. No. 8,781,571.
Application No. 13/249,975 is a continuation-in-part of application No. 15/181,166, filed on Jun. 13, 2016.
Application No. 15/181,166 is a continuation of application No. 14/002,909, filed on Jan. 6, 2014, now Pat. No. 9,364,656.
Application No. 14/002,909 is a continuation of application No. 13/250,031, filed on Sep. 30, 2011, now Pat. No. 8,301,238.
Application No. 13/250,031 is a continuation-in-part of application No. 14/815,676, filed on Jul. 31, 2015, now Pat. No. 9,645,179.
Application No. 14/815,676 is a continuation of application No. 13/866,371, filed on Apr. 19, 2013, now Pat. No. 9,095,706.
Application No. 13/866,371 is a division of application No. 13/476,960, filed on May 21, 2012, now Pat. No. 8,428,708.
Application No. 13/476,960 is a continuation-in-part of application No. 14/406,969, filed on Dec. 10, 2014.
Application No. 14/406,969 is a continuation of application No. 13/493,314, filed on Jun. 11, 2012, now Pat. No. 8,428,709.
(60) Provisional application No. 61/470,340, filed on Mar. 31, 2011.
(60) Provisional application No. 61/470,352, filed on Mar. 31, 2011.

(51) Int. Cl. A61N 1/32; A61K 31/4468; B29C 65/48; B29C 65/56; A61N 1/08; A61M 37/00

(52) U.S. Cl. CPC A61N 1/325; A61N 1/327; A61N 1/08; A61M 37/00; B29C 65/48; B29C 65/56; A61K 31/4468; A61M 2207/00; A61M 2205/18; A61M 2205/502; A61M 2205/50; A61M 2205/587; A61M 2205/581; A61M 2205/702; A61M 2205/273; A61M 2205/3317; A61M 2037/0007; H01H 13/14

(57) ABSTRACT

A switch-operated therapeutic agent delivery device. Embodiments of the operated therapeutic agent delivery device my include a switch that can be operated by a user, a device controller connected to the switch through a switch input where the device can actuate the device when certain predetermined conditions are met, following performance of both a digital switch validation test and an analog switch validation test. The switch operated therapeutic agent delivery device may have two parts, which are assembled by a user prior to use. These devices may be configured to determine if a current is present between the anode and cathode when drug is not intended to be delivered by the device. These devices may indirectly control and/or monitor the applied current without directly measuring from the cathode of the patient terminal.

### CROSS REFERENCE TO RELATED APPLICATION

This application is a continuation-in-part of U.S. patent application Ser. No. 14/296,085, filed Jun. 4, 2014, which is a continuation-in-part to U.S. patent application Ser. No. 13/249,975, filed Sep. 30, 2011, now U.S. Pat. No. 8,781,571, which claims the benefit under 35 U.S.C. §119 of U.S. Provisional Patent Application No. 61/470,340, filed Mar. 31, 2011, each of which is herein incorporated by reference in its entirety.

This application is also a continuation-in-part of U.S. patent application Ser. No. 15/181,166, filed Jun. 13, 2016, which is a continuation of U.S. patent application Ser. No. 14/002,909, filed Jan. 6, 2014, now U.S. Pat. No. 9,364,656, which is a national stage filing under 35 U.S.C. §371 of PCT/US2012/028400, filed on Mar. 9, 2012, which claims priority to U.S. Provisional Application No. 61/470,352, filed Mar. 31, 2011, and also to U.S. patent application Ser. No. 13/250,031, filed Sep. 30, 2011, now U.S. Pat. No. 8,301,238, each of which is herein incorporated by reference in its entirety.

This application is a continuation-in-part of U.S. patent application Ser. No. 14/815,676, filed Jul. 31, 2015, which is a continuation of U.S. patent application Ser. No. 13/866,371, filed Apr. 19, 2013, now U.S. Pat. No. 9,095,706, which is a divisional of U.S. patent application Ser. No. 13/476,960, filed May 21, 2012, now U.S. Pat. No. 8,428,708, each of which is herein incorporated by reference in its entirety.

This application is a continuation-in-part of U.S. patent application Ser. No. 14/406,969, filed Dec. 10, 2014, which is a national stage filing under 35 U.S.C. §371 of PCT/US2013/029114, filed Mar. 5, 2013, which is a continuation of U.S. patent application Ser. No. 13/493,314, filed Jun. 11, 2012, now U.S. Pat. No. 8,428,709, each of which is herein incorporated by reference in its entirety.

### FIELD

The present invention relates generally to electrotransport drug delivery devices and methods of operation and use. These drug delivery devices may have improved safety. In particular, the invention is directed to drug delivery devices including automated self-testing.

### BACKGROUND

A switch-operated therapeutic agent delivery device can provide single or multiple doses of a therapeutic agent to a patient by activating a switch. Upon activation, such a device delivers a therapeutic agent to a patient. A patient-controlled device offers the patient the ability to self-administer a therapeutic agent as the need arises. For example, the therapeutic agent can be an analgesic agent that a patient can administer whenever sufficient pain is felt.

One means of patient controlled analgesia is patient controlled intravenous infusion, which is carried out by an infusion pump, which is pre-programmed to respond to the instructions of a patient within certain pre-determined dosing parameters. Such intravenous infusion pumps are commonly used for control of postoperative pain. The patient initiates infusion of a dose of analgesic, which is typically a narcotic, by signaling a control unit. The unit receives the signal and, if certain conditions are met, begins infusion of the drug through a needle that has been inserted into one of the patient's veins.

Another form of patient controlled analgesia is electrotransport (e.g., iontophoresis, also referred to as iontophoretic drug delivery). In electrotransport drug delivery, a therapeutic agent is actively transported into the body by electric current. Examples of electrotransport include iontophoresis, electroosmosis and electroporation. Iontophoresis delivery devices typically comprise at least two electrodes connected to reservoirs, a voltage source, and a controller that controls delivery of the therapeutic agent by applying the voltage across the pair of electrodes. Usually at least one of the reservoirs contains a charged therapeutic agent (drug), while at least one reservoir contains a counter-ion and no therapeutic agent. The therapeutic agent, which is a charged species, is driven from the reservoir containing the therapeutic agent and into and across the skin into the patient to whom the reservoirs are attached.

In addition to therapeutic agent, the reservoirs may contain other charged and uncharged species. For example, the reservoirs are often hydrogels, which contain water as a necessary constituent. The reservoirs may also contain electrolytes, preservatives, antibacterial agents, and other charged and uncharged species.

For safety reasons, it is essential that any patient-controlled drug delivery device, and particularly an electrotransport device delivering a therapeutic agent (e.g., an opoid analgesic such as fentanyl) be tightly regulated to prevent the inadvertent delivery of agent to a patient. For example, short circuits in the device may result in erroneous, additional delivery of drug. Since patient-activated dosing systems must include a dose switch that is selected, e.g., pushed, by a patient to deliver a dose, one particularly vulnerable aspect is this switch. A short circuit in the dose switch circuit could be interpreted by control logic (e.g., processor) of the device as valid dose switch presses, and potentially cause the system to deliver a dose even without a valid patient request. Such short circuits could be caused by contamination or corrosion.

Described herein are methods and apparatuses (e.g., system and devices) that validate the integrity of a dose switch circuit and signal characteristics prior to initiating a dose. In particular, the apparatuses and methods described herein perform validation before each dose initiation, and the validation process (e.g., measurements used to determine if the switch is properly functioning) do not interfere with normal operation, including in particular actual presses of the dose switch. The apparatus and methods described herein are demonstrably reliable to a high degree of certainty. These apparatus and methods may therefore address the issues raised above.

The delivery of active pharmaceutical agents through the skin provides many advantages, including comfort, convenience, and non-invasiveness. This technology may also avoid gastrointestinal irritation and the variable rates of absorption and metabolism, including first pass effects, encountered in oral delivery. Transdermal delivery can also provide a high degree of control over blood concentrations of any particular active agent.

Transdermal delivery of active agents may involve the use of electrical current to actively transport the active agent into the body through intact skin by electrotransport. Electrotransport techniques may include iontophoresis, electroosmosis, and electroporation. Electrotransport devices, such as iontophoretic devices are known in the art. See, e.g., U.S. Pat. No. 6,216,033 B1 (Southam, et al.) One electrode, which may be referred to as the active or donor electrode, is the electrode from which the active agent is delivered into the body. The other electrode, which may be referred to as the counter or return electrode, serves to close the electrical circuit through the body. In conjunction with the patient's body tissue, e.g., skin, the circuit is completed by connection of the electrodes to a source of electrical energy, and usually to circuitry capable of controlling the current passing through the device. If the substance to be driven into the body is ionic and is positively charged, then the positive electrode (the anode) will be the active electrode and the negative electrode (the cathode) will serve as the counter electrode. If the ionic substance to be delivered is negatively charged, then the cathodic electrode will be the active electrode and the anodic electrode will be the counter electrode.

A switch operated therapeutic agent delivery device can provide single or multiple doses of a therapeutic agent to a patient by activating a switch. Upon activation, such a device delivers a therapeutic agent to a patient. A patient-controlled device offers the patient the ability to self-administer a therapeutic agent as the need arises. For example, the therapeutic agent can be an analgesic agent that a patient can administer whenever sufficient pain is felt.

There have been suggestions to provide different parts of an electrotransport system separately and connect them together for use. For example, it has been suggested that such connected-together systems might provide advantages for reusable controller circuit. In reusable systems, the drug-containing units are disconnected from the controller when the drug becomes depleted and a fresh drug-containing unit is then connected to the controller again. Examples of electrotransport devices having parts being connected together before use include those described in U.S. Pat. No. 5,320,597 (Sage, Jr. et al); U.S. Pat. No. 4,731,926 (Sibalis), U.S. Pat. No. 5,358,483 (Sibalis), U.S. Pat. No. 5,135,479 (Sibalis et al.), UK Patent Publication GB2239803 (Devane et al), U.S. Pat. No. 5,919,155 (Lattin et al.), U.S. Pat. No. 5,445,609 (Lattin et al.), U.S. Pat. No. 5,603,693 (Frenkel et al.), WO1996036394 (Lattin et al.), and U.S. 2008/0234628 A1 (Dent et al.).

There remain issues to be resolved and problems to be overcome in the art of electrotransport of therapeutic agents. The methods and apparatuses described herein may address these issues.

The consequences of delivering an inappropriate dosage (e.g., too much or too little) of a drug can be life threatening, thus it is of critical importance that drug delivery systems be extremely accurate. Drug delivery systems that are configured to deliver medication to patients must be configured to prevent even unlikely accidental delivery events. In particular, drug delivery systems that electrically deliver drug to a patient, including transdermal or other electroransport drug delivery devices, should ideally prevent accidentally providing drug to the patient.

The term “electrotransport” as used herein refers generally to the delivery of an agent (e.g., a drug) through a biological membrane, such as skin, mucous membrane, or nails. The delivery is induced or aided by application of an electrical potential. For example, a beneficial therapeutic agent may be introduced into the systemic circulation of a human body by electrotransport delivery through the skin. A widely used electrotransport process, electromigration (also called iontophoresis), involves the electrically induced transport of charged ions. Another type of electrotransport, electro-osmosis, involves the flow of a liquid. The liquid contains the agent to be delivered, under the influence of an electric field. Still another type of electrotransport process, electroporation, involves the formation of transiently-existing pores in a biological membrane by the application of an electric field. An agent can be delivered through the pores either passively (i.e., without electrical assistance) or actively (i.e., under the influence of an electric potential). However, in any given electrotransport process, more than one of these processes may be occurring simultaneously to a certain extent. Accordingly, the term “electrotransport”, as used herein, should be given its broadest possible interpretation so that it includes the electrically induced or enhanced transport of at least one agent, which may be charged, uncharged, or a mixture thereof, regardless of the specific mechanism or mechanisms by which the agent is transported.

In general, electrotransport devices use at least two electrodes that are in electrical contact with some portion of the skin, nails, mucous membrane, or other body surface. One electrode, commonly called the “donor” or “active” electrode, is the electrode from which the agent is delivered into the body. The other electrode, typically termed the “counter” or “return” electrode, serves to close the electrical circuit through the body. For example, if the agent to be delivered is positively charged, i.e., a cation, then the anode is the active or donor electrode, while the cathode serves to complete the circuit. Alternatively, if an agent is negatively charged, i.e., an anion, the cathode is the donor electrode. Additionally, both the anode and cathode may be considered donor electrodes if both anionic and cationic agent ions, or if uncharged dissolved agents, are to be delivered.

Furthermore, electrotransport delivery systems generally require at least one reservoir or source of the agent to be delivered to the body. Examples of such donor reservoirs include a pouch or cavity, a porous sponge or pad, and a hydrophilic polymer or a gel matrix. Such donor reservoirs are electrically connected to, and positioned between, the anode or cathode and the body surface, to provide a fixed or renewable source of one or more agents or drugs. Electrotransport devices also have an electrical power source such as one or more batteries. Typically, one pole of the power source is electrically connected to the donor electrode, while the opposite pole is electrically connected to the counter electrode. In addition, some electrotransport devices have an electrical controller that controls the current applied through the electrodes, thereby regulating the rate of agent delivery. Passive flux control membranes, adhesives for maintaining device contact with a body surface, insulating members, and impermeable backing members are some other potential components of an electrotransport device that may be used.

Small, self-contained electrotransport drug delivery devices adapted to be worn on the skin for extended periods of time have been proposed. See, e.g., U.S. Pat. No. 6,171,294, U.S. Pat. No. 6,881,208, U.S. Pat. No. 5,843,014, U.S. Pat. No. 6,181,963, U.S. Pat. No. 7,027,859, U.S. Pat. No. 6,975,902, and U.S. Pat. No. 6,216,033. These electrotransport agent delivery devices typically utilize an electrical circuit to electrically connect the power source (e.g., a battery) and the electrodes. The electrical components in such miniaturized iontophoretic drug delivery devices are also preferably miniaturized, and may be in the form of either integrated circuits (i.e., microchips) or small printed circuits. Electronic components, such as batteries, resistors, pulse generators, capacitors, etc., are electrically connected to form an electronic circuit that controls the amplitude, polarity, timing waveform shape, etc., of the electric current supplied by the power source. Other examples of small, self-contained electrotransport delivery devices are disclosed in U.S. Pat. No. 5,224,927; U.S. Pat. No. 5,203,768; U.S. Pat. No. 5,224,928; and U.S. Pat. No. 5,246,418.

One concern, particularly with small self-contained electrotransport delivery devices which are manufactured with the drug to be delivered already in them, is the potential for unintended delivery of drug because of electrical energy applied from an outside source, or because of an internal short. Any current or potential difference between the anode and cathode of the device may result in delivery of drug by a device contacting the skin, even if the device is not activated or in an off state. For example, drug may unintentionally be delivered if a current is applied through the devices or to a subject wearing a device, even if the device is in an off mode (even powered off). This risk, while hopefully unlikely, has not previously been addressed by electrotransport drug delivery devices.

Although an electrotransport device may include control circuitry and/or modules (e.g., software, firmware, hardware, etc.) configured specifically to regulate the current (and therefore the dosage of drug) applied when the device is “on,” such devices do not typically monitor the devices when they are in an “off” state.

Described herein are methods, devices and systems for monitoring and controlling electrotransport drug delivery devices to detect and/or prevent delivery of drug by the device when it is in an off mode or state. In particular, described herein are devices, systems and methods that confirm that voltage or current is not applied between the electrodes (anode and cathode) of the device when it is in an “off” state or mode.

In some variations it may be beneficial to control and monitor the applied current without directly monitoring the second patient terminal (e.g., cathode). This configuration allows separation of the control aspect of the circuit from the risk management aspect of the circuitry.

For example, also described herein are methods, devices and systems for monitoring and controlling electrotransport drug delivery devices including indirectly monitoring and controlling the circuit not directly connected to the patient terminal (e.g., cathode) using a switching element.

### SUMMARY OF THE DISCLOSURE

The present invention addresses a need in the art of patient-controlled drug administration devices, especially those devices that are subject to humidity and other contaminants during storage and use, such as iontophoresis devices. The inventors have identified contaminants present in storage and use of iontophoresis devices, as being particularly problematic, as they can cause the device to malfunction. For example, in electrotransport, such as iontophoresis—and on-demand drug delivery in general—faulty circuitry can be especially problematic, as it can, in some instances, cause the device to fail to deliver a full dose, to deliver more than the desired dose, to deliver one or more doses during storage, to deliver one or more doses in the absence of a patient instruction, etc. The potential for contamination of electronic circuitry is especially present in iontophoretic drug delivery systems, as the reservoirs employed contain water as well as other charged and uncharged species—such as charged therapeutic agent, electrolytes, preservatives and antibacterial agents—which can contaminate circuitry, such as activation switches, circuit leads, circuit traces, etc. (Other drug delivery methods, such as patient-activated pumps, can present similar potential for contamination, especially with environmental humidity and airborne contaminants.) In combination with voltages and currents applied to the circuitry during drug delivery (and in some cases storage), contaminants can cause current leaks, short circuits (“shorts”, including intermittent shorts) and other spurious signals that can interfere with the proper operation of the device. Other causes of circuit malfunction can also be introduced during manufacturing or in the use environment. The inventors have identified a particular part of the circuitry—the activation switch, as a point that is in some cases especially vulnerable to contamination and malfunction. The inventors have further identified the activation switch as a part of the circuitry that is a focal point for detecting and averting potential and actual circuit faults before they negatively impact device performance, and ultimately, patient health.

Embodiments of the device and methods described herein address the issues raised above by providing means to actively seek out and detect circuit faults and precursors to faults. The means employed involve performing active checks of the device circuitry while the device is powered on, e.g. before, during or after drug delivery. Some embodiments of the device and methods described herein provide for active detection of circuit faults and/or precursors to faults after any button push or after any event that mimics a button push, such as a spurious voltage. Some embodiments provide for active detection of circuit faults or precursors to faults, for instance, between button pushes in an activation sequence, during drug delivery, and between drug delivery sequences (i.e. after one dose has been delivered and before commencement of delivery of another dose).

In some embodiments, the active testing during use of the device is in addition to testing during or following device manufacturing.

Thus there is described herein are therapeutic agent delivery devices, such as electrotransport device (e.g. an iontophoresis device), which may include a housing and components adapted for containing and delivering the therapeutic agent to a patient, a processor for controlling delivery of the therapeutic agent to the patient, and circuitry and/or control logic for detecting one or more faults and/or precursors to faults during device operation, and for disabling the device upon detection of a fault or a precursor to a fault. In some embodiments, the device is an iontophoresis device or other electrotransport device. In some embodiments, the device further comprises an alarm for alerting a patient and/or caregiver that the device has detected a fault and/or precursor to a fault. In some embodiments, the device further comprises an alarm for alerting a patient and/or caregiver that the device is being disabled. In some embodiments, the either or both alarms are at least one of: an audible tone (or tones), at least one visual indicator, or a combination of two or more thereof. In some embodiments, the means for containing and delivering therapeutic agent to the patient includes one or more therapeutic agent reservoirs connected to one or more electrodes for applying a current to the reservoirs and actively transporting therapeutic agent across an outer surface of a patient, such as the skin. In some embodiments, the means for detecting a fault or a precursor to a fault is configured to detect a fault in a switch, such as an activation switch, or other circuit component, such as a trace, a connector, a power supply, an integrated circuit, a lead, a chip, a resistor, a capacitor, an inductor or other circuit component. In some embodiments the means for controlling delivery of the therapeutic agent comprises a pre-programmed or programmable integrated circuit controller, such as an ASIC.

In some embodiments, the circuitry described herein is incorporated into a device for delivery of a therapeutic agent (drug) to a patient. In some embodiments, the device is a patient-activated drug delivery device. In some embodiments, the device is an electrotransport drug delivery device. In some embodiments, the drug delivery device is an iontophoretic drug delivery device. In some embodiments, the drug to be delivered is an opioid analgesic. In some embodiments, the opioid analgesic is a pharmaceutically acceptable salt of fentanyl or sufentanil, such as fentanyl hydrochloride.

In some embodiments, the methods described herein are executed by a device processor, which may include or be referred to as a controller, especially a controller of a device for delivery of a therapeutic agent (drug) to a patient. In some embodiments, the methods are carried out by the controller during one or more stages of drug delivery—e.g., during the period of time between pushes of an activation button, during delivery of the drug, between delivery sequences, etc. In some preferred embodiments, the testing is carried out after any button push or anything that appears to be a button push. In particularly preferred embodiments, the methods are under active control of the controller, meaning that the controller initiates detection of faults and precursors to faults in the circuitry, e.g. after a button push or anything that appears to be a button push. In some embodiments, upon detection of a fault or precursor to a fault, the controller takes appropriate action, such as setting a fault detection flag, logging the fault in memory for retrieval at a later time, setting a user warning (such as an indicator light and/or audible tone), and/or disabling the device. In this regard, methods for disabling a device upon detection of a fault are described in U.S. Pat. No. 7,027,859 to McNichols et al., which is incorporated herein in its entirety; in particular column 6, line 65 through column 11, line 35 are specifically incorporated by reference as teaching various ways to disable a circuit.

Described herein are switch operated devices, such as a drug delivery device (e.g., a drug delivery pump or iontophoresis device) comprising: (a) a device switch configured to be operated by a user, which provides a switch signal to a switch input of a device controller when operated by a user; (b) the device controller, having said switch input operatively connected to the switch, and configured to receive the switch signal from the switch, the device controller being configured to actuate the device when the switch signal meets certain predetermined conditions and to control and receive signals from a switch integrity test subcircuit; and (c) the switch integrity test subcircuit, which is configured to detect a fault or a precursor to a fault in the switch and provide a fault signal to the controller. When the controller receives a fault signal from the switch integrity test subcircuit, it executes a switch fault subroutine when a fault or a precursor to a fault is detected. In some embodiments, the switch integrity test subcircuit is configured to check for and detect a fault or a precursor to a fault in the switch. In some embodiments, the switch integrity test subcircuit is configured to test for and detect at least one fault or precursor to a fault such as contamination, short circuits, (including intermittent short circuits), compromised circuit components (including malfunctioning resistors, integrated circuit pins, and/or capacitors), etc.

In some embodiments, the switch integrity test subcircuit is configured to test for and detect a voltage (or change in voltage) between the switch input and ground or some intermediate voltage above ground, a short between the switch input and a voltage pull up or some intermediate voltage below the pull up voltage. In some preferred embodiments the switch integrity test subcircuit is configured to test for and detect a voltage (or change in voltage) between the switch input and some intermediate voltage above ground (a low voltage, V_{L}) and/or a short between the switch input and a some intermediate voltage below the pull up voltage (high voltage V_{H}). Thus, the switch integrity test subcircuit is able to detect a non-determinant signal that indicates contamination (e.g. moisture and/or particulates), corrosion, a damaged circuit resistor, a damaged integrated circuit pin, etc. In some embodiments, the switch fault subroutine includes at least one of: activating a user alert feature, logging detection of faults or precursors to faults, deactivating the device, or one or more combinations thereof. In some embodiments, the controller is configured to measure a voltage or a rate of change of voltage at the switch input and execute the switch fault subroutine when the voltage or rate of change of voltage at the switch input fails to meet one or more predetermined parameters. In some embodiments, the device is an iontophoresis delivery device comprising first and second electrodes and reservoirs, at least one of the reservoirs containing therapeutic agent to be delivered by iontophoresis. In some embodiments, the predetermined conditions for actuating the device include the user activating the switch at least two times within a predetermined period of time. In some embodiments, the switch input is pulled up to a high voltage when the switch is open and the switch input is a low voltage when the switch is closed.

Some embodiments described herein provide a method of switch fault detection in a switch operated device, said device comprising: (a) a device switch connected to a switch input of a device controller; (b) the device controller comprising said switch input; and (c) a switch integrity test subcircuit, said method comprising said controller: (i) activating the switch integrity test subcircuit; (ii) detecting a voltage condition at the switch input; and (iii) activating a switch fault subroutine if the voltage condition at the switch input fails to meet one or more predetermined conditions. In some embodiments, the steps of activating the switch integrity test subcircuit and detecting a voltage condition at the switch input are executed continuously or periodically throughout use of the device. In some embodiments, the switch fault subroutine includes, for example, activating a user alert feature, logging detection of faults or precursors to faults, deactivating the device, or one or more combinations thereof. In some embodiments, the voltage condition is a voltage, a change in voltage or both. In some embodiments, the controller detects the voltage at the switch input under conditions in which the voltage should be zero or nearly zero if the switch integrity is within operating norms, and activates the switch fault subroutine if the voltage is significantly higher than zero. In some embodiments, the controller detects the voltage at the switch input under conditions in which the voltage should be equal to a pull up voltage or nearly equal to the pull up voltage if the switch integrity is within operating norms, and activates the switch fault subroutine if the voltage is significantly lower than the pull up voltage. In some embodiments, the controller detects a change in voltage at the switch input under conditions in which the voltage is expected to fall to zero or nearly to zero after within a predetermined period if the switch integrity is within operating norms, and activates the switch fault subroutine if the voltage fails to fall to zero or nearly to zero within the predetermined period. In some embodiments, the controller detects a change in voltage at the switch input under conditions where, the voltage should rise to a pull up voltage or nearly to the pull up voltage within a predetermined period if the switch integrity is within operating norms, and activates the switch fault subroutine if the voltage fails to rise to the pull up voltage or nearly to the pull up voltage within the predetermined period.

Some embodiments described herein provide a switch operated iontophoresis therapeutic agent delivery device, comprising: (a) a power source; (b) first and second electrodes and reservoirs, at least one of the reservoirs containing the therapeutic agent; (c) a device switch, which provides a switch signal to a switch input of a device controller when operated by a user, the device controller, having said switch input operatively connected to the switch, whereby the controller receives the switch signal from the switch, the device controller being operatively connected to a power source that provides power to the first and second electrodes for delivering therapeutic agent to a patient; and (d) a switch integrity test subcircuit, which is configured to detect a fault in the switch and cause the controller to execute a switch fault subroutine when a fault is detected. In some embodiments, the therapeutic agent is an opioid analgesic as described herein, such as fentanyl or sufentanil or a pharmaceutically acceptable salt, analog or derivative thereof.

A method of switch fault detection in a user operated iontophoresis therapeutic agent delivery device, said device comprising: (a) a power source; (b) first and second electrodes and reservoirs, at least one of the reservoirs containing the therapeutic agent; (c) a device switch connected to a switch input of a device controller; (d) the device controller comprising said switch input and configured to control power to the first and second electrodes, thereby controlling delivery of the therapeutic agent; and (e) a switch integrity test subcircuit, said method comprising said controller: (i) activating the switch integrity test subcircuit; detecting a voltage condition at the switch input; and (ii) activating a switch fault subroutine if the voltage condition at the switch input fails to meet one or more predetermined conditions. In some embodiments, the switch fault subroutine includes, for example, activating a user alert, deactivating the device, or both.

Also described herein are methods of validating the operation of a switch including a user-activated to deliver a dose of a drug from a drug delivery device. Any of the drug delivery devices described herein may be transdermal drug delivery devices. A method of validating the operation of a switch (e.g., a user-activated switch) to deliver a dose of drug from a (e.g., transdermal) drug delivery device may include: monitoring the switch to determine a release event; performing a digital validation of the switch following the release event; performing an analog validation of the switch following the release event; and initiating a failure mode for the drug delivery device if the analog validation of the switch fails.

In general, the methods of validating the operation of a switch and apparatus configured to validate the operation of a switch may include button sampling when monitoring the switch. For example, monitoring the switch may generally include sequentially sampling a switch input, storing a window of sequential samples, and comparing a plurality of more recent sequential samples to a plurality of older sequential samples within the stored window of samples to detect the release event. Sequential sampling may refer to periodically sampling an input to the switch (e.g., the low or high side of the switch) at regular intervals, e.g., every 1 ms, 2 ms, 3 ms, 4 ms, 5 ms, 6 ms, 7 ms, 8 ms, 9 ms, 10 ms, etc. The plurality of more recent sequential samples may refer to 2 or more, 3 or more, 4 or more, 5 or more, etc., samples taken sequentially in time. The window of stored sequential samples may be a circular buffer, storing a rolling window of samples (e.g., any appropriate number of samples may be stored, with the most recent sample replacing the oldest sample in a continuous manner). Thus, in general, a group of newer sequential samples may be compared to a group of older sequential samples and if the state change is made (e.g., when the older samples all indicate the switch is closed, and the newer samples all indicate the switch is open, a release event may be confirmed. For example, monitoring the switch to determine a release event may include sequentially sampling a switch input, storing a window of sequential samples, and comparing three or more recent sequential samples to three or more older sequential samples within the stored window of samples to detect the release event, e.g., when the three or more recent samples indicate an open switch and the three or more older samples indicate a closed switch. The older samples and the more recent samples are generally non-overlapping.

In general, the failure mode, as discussed above, may include suspending operation of the device, shutting the device off, or restarting the device. For example, the failure mode may include preventing delivery of drug by the device, including (but not limited to) turning off the drug delivery device, and/or locking (e.g., inactivating) the drug delivery device.

In general, both digital and analog validation tests may be performed on the switch, typically during a period when the switch is reliable predicted to be in the “open” (inactivated) state. The inactivated state is known most reliably immediately or shortly (e.g. within micro- to mili-seconds) following user activation, as it may be impossible for a user to more quickly activate the switch immediately after one (or better yet, a series) of “pushes” or other activating input. Thus, in variations in which the user pushes a button (activates the switch) multiple times, e.g., twice, within a predetermined activation period (e.g., two quick ‘clicks’ in succession), during the period (e.g., between about 8 μsec and 500 msec, between about 8 μsec and 400 msec, between about 8 μsec and 300 msec, between about 8 μsec and 200 msec; less than about 500 msec, less than about 400 msec, less than about 300 msec, less than about 200 msec, less than about 150 msec, less than about 100 msec, etc.) it is unlikely that the user would validly activate the switch, and therefore the state of the switch should be in the open state. Thus, both the analog and digital validation may be performed within this period, which may be referred to as a test period or test window.

Analog validation of the switch typically means determining the actual voltage value of one or both sides of the switch and comparing them to one or more thresholds to confirm that they are within acceptable parameters. For example, performing the analog validation of the switch may comprise performing an analog validation of the switch if the digital validation passes. Either or both digital and analog validation may include performing the analog validation using a dose switch circuit. The dosing switch circuit may be part of the processor/controller.

In general, method or apparatus may perform the digital validation and analog validation sequentially or in parallel. For example, the digital validation step may be performed before the analog validation step; the analog validation step may be performed only if the digital validation passes (e.g., does not fail digital validation); the drug delivery apparatus may be re-started (e.g., the button sampling process may be re-started) if the digital validation of the switch fails.

The digital validation generally includes a comparison of the logical values of digital validation lines from one or both sides of the switch to expected values based on the inputs from the power source (e.g., battery) to the switch. For example, digital validation may “fail” (e.g., failing the digital validation) if a secondary digital input on a first side of the switch does not match a primary digital input on the first side of the switch, or a secondary digital input on a second side of the switch does not match a primary digital input on the second side of the switch. The primary digital input may be a first input line connected to the battery and the high side of the switch and the secondary digital input may be a second input line connected to the patter and the low side of the switch. The secondary digital input line may be a first digital test input line also connected on the high side of the switch. Similarly, the analog validation may be performed using a first and second analog input line; the first analog test input line may be on the high side of the switch and the second analog test input line may be on the low side of the switch.

Performing the digital validation may include failing the digital validation if a secondary digital input on a high side of the switch is low or if a secondary digital input on a low side of the switch is high.

Performing the analog validation may include failing the analog validation if a measurement of a high side voltage is less than a first predetermined fraction (e.g., 90%, 85%, 80%, 75%, 70%, 65%, etc.) of a battery voltage for the drug delivery device, or a measurement of a low side voltage is greater than a second predetermined fraction (e.g., 90%, 85%, 80%, 75%, 70%, 65%, etc.) of the battery voltage. For example, performing the analog validation may include failing the analog validation if a measurement of a high side voltage is less about 0.8 times a battery voltage for the drug delivery device, or a measurement of a low side voltage is greater than about 0.2 times the battery voltage. Performing the analog validation may include sequentially measuring a high side voltage and a low side voltage using an analog to digital converter (ADC) and failing the analog validation if the high side voltage is below a first predetermined threshold or the low side voltage is above a second predetermined threshold.

As mentioned, digital validation of the switch may be performed before the analog validation of the switch. Alternatively, analog validation of the switch may be performed before the digital validation of the switch.

In general, a release event may include a second release of the switch within a predetermined time period. For example, a release event may comprise a second release of the switch within less than about 400 msec, 300 msec, 200 msec, 100 msec, etc.

For example, a method of validating operation of a switch, wherein the switch is user-activated to deliver a dose of a drug from a drug delivery device, may include: monitoring the switch to determine a release event; performing a digital validation of the switch following the release event using a dose switch circuit and failing the digital validation if a secondary digital input on a high side of the switch is low or if a secondary digital input on a low side of the switch is high; performing an analog validation of the switch if the digital validation passes and failing the analog validation if a measurement of a high side voltage is less than a first predetermined fraction of a battery voltage for the drug delivery device or if a measurement of a low side voltage is greater than a second predetermined fraction of the battery voltage; and initiating a failure mode for the drug delivery device if the analog validation of the switch fails.

Any of the drug delivery devices described herein may be adapted to validate the operation of a user-selectable activation switch to deliver a dose of drug. For example a drug delivery device may include: a battery having a battery voltage; a switch configured to be activated by a user to deliver a dose of drug, the switch having a low voltage side and a high voltage side; a first input line on the high side and a second input line on the low side, wherein the first and second input lines are connected to the battery; a first analog test input line on the high side and a second analog test input line on the low side; a first digital test input line on the high side and a second digital test input line on the low side; and a controller configured to perform a digital validation of the switch following a release event of the switch and to perform an analog validation of the switch following the release event, wherein the controller is further configured to initiate a failure mode for the drug delivery device if the analog validation of the switch fails.

In general, any of these devices may include a circular buffer configured to store a plurality of sequential samples from an input line on the low voltage side of the switch, wherein the newest sample replaces the oldest sample.

Further, the controller may be configured determine a release event on the switch by being configured to sequentially sample an input line on the high voltage side of the switch, store a window of sequential samples, and compare a plurality of more recent sequential samples to a plurality of older sequential samples within the stored window of samples to detect the release event.

The first and second analog test input lines may be connected to the controller, and further wherein the controller configured to fail the analog validation if a voltage on the first analog test line is below a first predetermined fraction of the battery voltage or if a voltage on the second analog test line is greater than a second predetermined fraction of the battery voltage. For example, the first and second analog test input lines may be connected to the controller, and further wherein the controller configured to fail the analog validation if a voltage on the first analog test line is less about 0.8 times the battery voltage or if a voltage on the second analog test line is greater than about 0.2 time the battery voltage.

The first and second digital test input lines may be connected to the controller, wherein the controller is configured to fail the digital validation if a value of the first digital test input line does not match a value of the first input line or if a value of the second digital test input line does not match a value of the second input line. For example, the first and second digital test input lines may be connected to the controller, wherein the controller is configured to fail the digital validation if the first digital input line is low or if the second digital input line is high.

The controller may be configured to perform the analog validation of the switch and the digital validation of the switch following a second release of the switch within less than about 500 msec (e.g., less than about 400 msec, less than about 300 msec, less than about 200 msec, less than about 100 msec, etc.).

For example, a drug delivery device adapted to validate the operation of a user-selectable activation switch to deliver a dose of drug may include: a battery having a battery voltage; a switch configured to be activated by a user to deliver a dose of drug, the switch having a low voltage side and a high voltage side; a first input line on the high side and a second input line on the low side, wherein the first and second input lines are connected to the battery; a first analog test input line on the high side and a second analog test input line on the low side, wherein the first and second analog test inputs lines are connected to a controller; and a first digital test input line on the high side and a second digital test input line on the low side, wherein the first and second digital test input lines are connected to the controller; wherein the controller is configured to perform a digital validation of the switch, following a second release of the switch within a predetermined time period, and to perform an analog validation of the switch following the second release of the switch within the predetermined time period, further wherein the controller is configured to fail the analog validation if a voltage on the first analog test line is below a first predetermined fraction of the battery voltage or if a voltage on the second analog test line is greater than a second predetermined fraction of the battery voltage, and to fail the digital validation if the first digital input line is low or if the second digital input line is high; and wherein the controller initiates a failure mode for the drug delivery device if the analog validation of the switch fails.

The present disclosure also describes a two-part electrotransport therapeutic agent delivery device, such as an iontophoresis device, in which the two parts of the device are provided separately and assembled to form a unitary, powered-on device at the point of use—e.g. just prior to use. One part of the device, which may be referred to herein as the electrical module, holds essentially all of the circuitry, as well as the power source (e.g. battery), for the device; and the other part, which may be referred to herein as the reservoir module, contains the therapeutic agent to be delivered along with electrodes and hydrogels necessary to deliver the therapeutic agent to a patient. The device is configured such that the power source is kept electrically isolated from the rest of the circuitry in the electrical module until the electrical module is combined with the reservoir module. The combination of the modules occurs in a single action by a user, along with connection of the battery into the circuitry. Thus, embodiments provided herein permit the combination of the electrical module and the reservoir module, whereby in a single action the two modules form a single unit and the battery is introduced into the circuitry, thereby powering on the device, in a single action by the user.

The present invention addresses various needs, and provides various advantages, in the art of patient-controlled drug administration devices, especially those devices that are subject to humidity and other contaminants during storage and use, such as iontophoresis devices. Electrical components, especially those that have electrical charges applied to them, are especially vulnerable to corrosion, particularly when they are exposed to humidity and/or contaminants, such as ions and particulate contaminants. By keeping the electrical circuitry isolated from the hydrogels in the reservoir module prior to use, the device described herein reduces the tendency of electronic circuitry to be corroded by humidity emitted from the hydrogels. In embodiments of the device described herein, not only is the electrical circuitry maintained in isolation from the water-containing reservoir module prior to use, thereby reducing water contamination of the circuitry, the battery itself is maintained in electronic isolation from the electronic circuitry prior to combination of the two modules. Thus, unlike previously devised electrotransport devices, which generally comprised a battery that was maintained in the electrical circuitry, embodiments of the device provided herein keep the battery out of the circuit until the two modules are combined, which prevents battery drain prior to use and prevents the circuitry from being subjected to electrostatic charges that can accelerate, or even cause, corrosion. In embodiments of the device provided herein, the two modules are combined (e.g. snapped) together and the battery is connected into the circuit in a single action by a user, such as a health care professional. In embodiments described herein, connection of the battery into the circuit turns the device “on” in the same single action. In some embodiments, once the device has been powered on, a controller or similar device runs one or more power-on checks to ensure that the device is in proper operating condition, and at least in some embodiments, signals a user that the device is ready for use. In certain embodiments, the controller or similar device is configured to detect an error state, such as a signal that indicates that the device is corroded, or an indication that the device has been previously used. In some such embodiments, the device then signals the user that an error has been detected (e.g. through a visual display or an audible alarm) and/or powers down. In some such embodiments, e.g. when the device is intended for a single use, once the device is powered down (e.g. by separating the two modules) the device will not again be operative.

In one aspect of the device described herein, the two parts (modules) are combined to form a single unit and the battery is connected into the circuitry, from which it has been previously electrically isolated, in a single action. Thus, there is no need to power the device on through some separate action, such as actuating a separate switch mechanism or removing a tab. Once the two modules are combined to for a single unit, the device is powered on and is enabled to perform the various functions that are required of it, such as running self diagnostics, receiving an activation signal from a user (e.g. a healthcare professional or patient) to effect drug delivery, and optionally powering off (e.g. at the end of its predetermined useful lifetime and/or upon detection of an error or other appropriate signal.)

In one aspect of the device described herein, the device is intended for single use. The device is configured to ensure that the electronic circuitry cannot be re-used, that is, the two modules may not be separated from one another and then rejoined to form an operative device, nor can the electrical module be combined with a different reservoir module to form an operative device. Such configuration includes single use (one way) couplers (e.g. single use snaps), electronic logic that detects and prevents an attempt to use the circuitry more than once (e.g. hardware, software, firmware, memory, etc., or a combination of two or more thereof), or various combinations thereof. In some embodiments, the device includes both mechanical and electrical means to prevent re-use.

In some embodiments, the device also includes one or more keying features designed to assist the user in combining the modules in a single configuration, which is the only operative configuration. Such keying features may include different sized couplers, variously shaped complementary external features of the modules, and visual alignment cues, or combinations of two or more thereof, which ensure that the user combines the two modules in the single, operative configuration only.

Some embodiments described herein provide an electrotransport drug delivery device comprising an electrical module and a reservoir module, the electrical module and the reservoir module being configured to be combined to form a unitary, activated drug delivery device prior to use, wherein: (a) the electrical module comprises: (i) circuitry; (ii) electrical outputs for connecting the circuitry to input connectors on the reservoir module when the electrical module is combined with the reservoir module; (iii) one or more power-on contacts between the circuitry and the battery; and (iv) a battery, which is isolated from the circuitry by the one or more power-on contacts while at least one of the power-on contacts remains open, and which is connected into the circuitry when each of the one or more power-on contacts is closed by one or more battery contact actuators on the reservoir module when the electrical module and the reservoir module are combined; and (b) the reservoir module comprises: (i) electrical inputs for electrically connecting the circuitry in the electrical module to at least a pair of active electrodes in the reservoir module when the electrical module is combined with the reservoir module; and (ii) one or more battery contact actuators, each of which is configured to close a corresponding power-on contact when the electrical module is combined with the drug reservoir, such that when each of the power-on contacts is closed by a power-on actuator, the battery is connected into the circuitry and the device is powered on. In some embodiments, at least one seal is formed upon combining the electrical module and the reservoir module. In some embodiments, at least one seal is maintained at each power-on contact before, during, and/or after the electrical module is combined with the reservoir module. In some embodiments, at least one seal is a flexible polymer cover over the power-on contact, which is configured to be deformed by an actuator when the electrical module is combined with the reservoir module, whereby the actuator mechanically acts through the seal to close the power-on contact. In some embodiments, at least one seal is maintained at each electrical output before, during, and after the electrical module is combined with the reservoir module. In some embodiments, at least one seal is water- or particulate-tight. In some embodiments, at least one seal is water-tight and particulate-tight. In some embodiments, the electrical outputs are configured to flex while continuously applying a force on the electrical inputs of the reservoir module to ensure good electrical connection between the two. In some embodiments, at least one surface of the electrical inputs is substantially planar. In some embodiments, the electrical module and the reservoir module are separately manufactured, packaged and/or shipped. In some embodiments, the electrical module and the reservoir module are configured to be combined to form a powered on drug delivery device just prior to attachment to a patient. In some embodiments, the device comprises one or more couplers on the reservoir module or the electrical module, each of which couples with a corresponding coupler receptor on the electrical module or reservoir module, respectively, to prevent the unitary drug delivery device from being easily separated. In some embodiments, each coupler is a snap, which is mechanically biased to snap into a corresponding snap receptor. In some embodiments, each snap is a one-way snap. In some embodiments, the device comprises two or more couplers and two or more corresponding coupler receptors. In some embodiments, at least two of the two or more couplers and two or more corresponding coupler receivers are of different sizes, whereby a first coupler can be inserted only into a first coupler receiver, thereby ensuring that the device can be assembled in only one configuration. In some embodiments, each coupler is biased so that once each coupler is engaged with its corresponding receptor, the device cannot be disassembled without breaking or deforming at least one of the couplers so that it is no longer operable. In some embodiments, the power-on contact is configured to be actuated by the battery contact actuator, thereby connecting the battery to the circuit, simultaneously, or substantially simultaneously, with coupling of the coupler and the coupler receptor. In some embodiments, one or more of the couplers and/or coupler receptors are water- and/or particulate-tight. In some embodiments, at least one water- and/or particulate-tight seal is formed between at least one coupler and at least one coupler receptor when they are coupled. In some embodiments, the battery contact actuator is a member, such as a post, that protrudes from the reservoir module and depresses a receptacle on the electrical module, the receptacle being in mechanical communication with the power-on contact such that the battery is connected into the circuit when the battery contact actuator depresses the receptacle. In some embodiments, the battery contact actuator is a post and the receptacle is a deformable member. In some embodiments, the deformable member is indented, flush or domed. In some embodiments, the device includes at least two power-on contacts and at least two corresponding battery contact actuators. In some embodiments, the battery is housed in a compartment that protrudes from the electrical module, which compartment has an outer shape that is configured to a corresponding indentation in the reservoir module such that the battery compartment fits snugly within the indentation in only one configuration when the electrical module and the reservoir module are combined to form the unitary device. In some embodiments, the electrical inputs on the reservoir module are flat or substantially flat electrically conductive metal, such as copper, brass, nickel, stainless steel, gold, silver or a combination thereof. In some embodiments, one or more of the electrical outputs includes one or more bumps protruding from electrical outputs. In some embodiments, the bumps are on one or more hats (described herein) protruding from the electrical module. In some embodiments, the hats are biased to maintain positive contact between the electrical outputs on the electrical module and the electrical inputs on the reservoir module. In some embodiments, the bias is provided by one or more springs or elastic members. In some embodiments, the bias is provided by one or more coil springs, beam springs or elastic members. In some embodiments, the device comprises one or more sealing members for providing a seal around the electrical inputs and outputs when the electrical module and the reservoir module are combined to form the unitary device. In some embodiments, the seal is a ring seal. In some embodiments, the seal is water- and/or particulate-tight. In some embodiments, the reservoir module is sealed in a container configured to be removed prior to combining the electrical module with the reservoir module to form the unitary device. In some embodiments, the container is a water- and/or particulate-tight pouch. In some embodiments, the electrical module further comprises a controller. In some embodiments, the controller is configured to execute a power-on check when the battery is connected into the circuitry. In some embodiments, the power-on check includes a battery test, an ASIC test, a power source test, an LCD check. In some embodiments, the device is configured to increment a logic flag when the electrical module is combined with the reservoir module, and wherein the device is configured such that, if the logic flag has met or exceeded a predetermined value, the device will either not power on or will power off if it has already powered on. In some embodiments, the device is configured to record an error code if the logic flag has met or exceeded a predetermined value. In some embodiments, the circuitry comprises a printed circuit board. In some embodiments, the one or more power-on contacts are configured to remove the battery from the circuitry if the electrical module and the reservoir module are separated after they have been combined. In some embodiments, the electrical module is configured to flex while maintaining a seal. In some embodiments, the seal is water- and/or particulate-tight. In some embodiments, the device further comprises an activation switch. In some embodiments, the device further comprises a liquid crystal diode (LCD) display, a light emitting diode (LED) display, an audio transducer, or a combination of two or more thereof.

Some embodiments described herein provide a method of drug delivery comprising: (a) combining an electrical module and a reservoir module to form a unitary powered-on drug delivery device, wherein: (i) the electrical module comprises: (1) circuitry; (2) electrical outputs for connecting the circuitry to input connectors on the reservoir module when the electrical module is combined with the reservoir module; (3) at least one power-on contact between the circuitry and the battery; and (4) a battery, which is isolated from the circuitry by the power-on contact until the power-on contact is actuated by a battery contact actuator on the reservoir module, and which is connected into the circuitry when the power-on contact is actuated by the battery contact actuator on the reservoir module when the electrical module and the reservoir module are combined; and (ii) the reservoir module comprises: (1) electrical inputs for electrically connecting the circuitry in the electrical module to at least a pair of active electrodes in the reservoir module when the electrical module is combined with the reservoir module; and (2) at least one battery contact actuator, which is configured to actuate said power-on contact when the controller module is combined with the drug delivery module, thereby connecting the battery into the circuitry; (b) applying the unitary device to a patient; and (c) activating the device to effect delivery of the drug to the patient.

Some embodiments described herein provide a process of manufacturing a drug delivery device, comprising: (a) assembling an electrical module comprising: (i) circuitry; (ii) electrical outputs for connecting the circuitry to input connectors on the reservoir module when the electrical module is combined with the reservoir module; (iii) at least one power-on contact between the circuitry and the battery; and (iv) a battery, which is isolated from the circuitry by the power-on contact until the power-on contact is actuated by a battery contact actuator on the reservoir module, and which is connected into the circuitry when the power-on contact is actuated by the battery contact actuator on the reservoir module when the electrical module and the reservoir module are combined; and (b) assembling a reservoir module comprising: (i) electrical inputs for electrically connecting the circuitry in the electrical module to at least a pair of active electrodes in the reservoir module when the electrical module is combined with the reservoir module; and (ii) at least one battery contact actuator, which is configured to actuate said power-on contact when the controller module is combined with the drug delivery module, thereby connecting the battery into the circuitry; and (c) packaging the electrical module and the reservoir module. In some embodiments, the process comprises sealing the reservoir module in a water- and/or particulate-tight pouch.

Also described herein are devices and methods including self-testing to prevent delivery of drug from an electrotransport drug delivery device when the device is not activated or in an off state.

For example, described herein are electrotransport drug delivery devices that prevent unwanted delivery of drug while in an off state. The device may include: an anode; a cathode; an activation circuit configured to apply current between the anode and cathode to deliver a drug by electrotransport when the device is in an on state and not in the off state; and an off-current module that is configured to automatically and periodically determine if there is a current flowing between the anode and cathode when the activation circuit is in the off state while powered on.

In general, the anode and/or cathode may connect to a source of the drug to be delivered, such as an analgesic like fentanyl and sufantanil within a gel matrix. The device may include a controller/processor or other electronic components (including software, hardware and/or firmware) forming the activation circuit and/or off-current module. In some variations the off-current module is integrated with other control systems (sub-systems) forming the device.

As used herein, a module, such as the off-current module, may include hardware, software, and/or firmware configured to perform the specified function (e.g., determine if a current is flowing between the anode and cathode). The module may include a combination of these, and may be a separate or separable region of the device or it may make use of shared components of the device (e.g., a microcontroller, resistive elements, etc.). For example, an off-current module comprises firmware, software and/or hardware configured to determine if there is a potential difference between the anode and the cathode when the activation circuit is in the off state while powered on. A module, such as the off-current module may include executable logic that operates on elements (e.g., a microcontroller) of the device. For example, the off-current module may include off-current monitoring logic controlling monitoring for the presence of a current (or indicator of current such as electrical potential, inductive or capacitive changes, etc.) between the anode and cathode when the device is otherwise in an off state.

In some variations of the device, systems and methods described herein the off-current module operates to monitor for and/or act upon identifying a current between the anode and cathode when the device is powered on but in an off state. Examples of off-states are provided below, but may include a ready state, a standby state, or the like, and may include any state during which the device is not in a dosing state and is not intended to deliver drug. The dosing state may be referred to as an on state and may indicate that the device is delivering drug. The off state described herein may occur when the device is otherwise powered on. In some variations, the off state includes the powered off state, while in some variations the off state does not include the powered off state, but only includes off states when the device is powered on.

In general, the off-current module may be configured to detect current flow between the anode and cathode in an off state either directly or indirectly. For example, in some variations the off-current module determines that current is flowing between the anode and cathode by monitoring for a voltage or a potential difference between the anode and cathode in the off state. For example, in some variations, the off-current module comprises software, firmware and/or hardware configured to determine if there is a change in capacitance between the anode and cathode when the activation circuit is in the off state while powered on. In one example an off-current module comprises software, firmware and/or hardware configured to determine if there is a change in inductance between the anode and cathode when the activation circuit is in the off state while powered on. Thus, current may be inferred to be flowing between the anode and cathode by monitoring indirectly for presence of or changes in potential difference (e.g., voltage), capacitance, inductance, or the like, between the anode and cathode of the device.

In general, the off-current module may indicate that current is flowing between the anode and cathode only when the detected current (or an indicator of current such as potential difference, inductance, capacitance, etc.) is above a threshold value. The threshold value is typically above the noise threshold for the device/system. This threshold may be predetermined. For example, in some variations the off-current module may comprise a sensing circuit that independently determines an anode voltage and a cathode voltage and compares the potential difference between the anode voltage and cathode voltage to a threshold value. For example, an off-current module may be configured to indicate that there is a current flowing between the anode and cathode when the activation circuit is in the off state while powered on where the current flowing is above an Output Current Off Threshold. Any appropriate Output Current Off Threshold may be used, e.g., about 1 μA, 3 μA, 5 μA, 9 μA, 10 μA, 15 μA, 25 μA, 30 μA, 50 μA, 100 μA, etc. In some variations the Output Current Off Threshold is about 9 μA.

An electrotransport device may include a switch connected between a reference voltage source and a sense resistor, so that the off-current module is configured to close the switch periodically to determine the potential difference between the anode voltage and cathode voltage.

Thus, in some variations the off-current module may be configured to determine if there is a potential difference between the anode and the cathode before the device allows current to travel through the anode and cathode. For example, the off-current module, be detecting if there is a current flowing between the anode and cathode even when the device is otherwise “off”, may trigger an alert that there is a leak current. In some variations the alert may include a shut-down of the device, and/or a visible (e.g., indicator light) or audible (e.g., beeping, buzzing, etc.) notification.

The off-current module may be configured to monitor at any periodic and/or automatic interval. For example, the off-current module may be configured to determine if there is a current flowing between the anode and cathode when the activation circuit is in the off state at least once per minute, once per 10 ms, once per 100 ms, once per 500 ms, once per 1 min, once per 2 min, once per 3 min, once per 4 min, once per 5 min, once per 10 min, once per 15 min, etc. For example, the off-current module may be configured to determine if there is a current flowing between the anode and cathode when the activation circuit is in the off state between at least once every 10 ms and once every 10 minutes.

In some variations an off-current module may be configured to wait some length of time (e.g., at least 10 ms) before determining if there is a current flowing between the anode and cathode when the activation circuit is in the off state. This length of time may be at least 4 ms, at least 10 ms, at least 15 ms, at least 30 ms, etc.

In some variations the electrotransport devices described herein have a two-part structure. The two-part structure may include: an electrical module including the activation circuit and the off-current module; and a reservoir module including the anode and the cathode and a source of drug to be delivered; wherein the electrical module and reservoir module are configured to be combined prior to application to a patient. In some variations, the off-current module may not be enabled until the electrical module and reservoir module are combined.

As mentioned above, in some variations, the off-current module may be configured to indicate that there is a current flowing between the anode and cathode when the activation circuit is in the off state while powered on, where the current flowing is above an Output Current Off Threshold. For example, the Output Current Off Threshold may be about 9 μA.

Also described herein are electrotransport drug delivery devices that prevent unwanted delivery of drug while in an off state. The device may include: a reservoir module including: an anode, a cathode and a source of drug; an electrical module including: an activation circuit configured to apply current between the anode and cathode to deliver a drug by electrotransport when the device is in an on state and not in the off state; and an off-current module, the module configured to automatically and periodically determine if there is a current flowing between the anode and the cathode greater than an Output Current Off Threshold of 9 μA when the activation circuit is in the off state while powered on; wherein the reservoir module and the electrical module are configured to be combined before being applied to a patient.

Methods of automatically and periodically confirming that drug will not be delivered by an electrotransport drug delivery device when the device is in an off state are also described herein. For example, a method of automatically and periodically confirming that drug will not be delivered by an electrotransport drug delivery device when the device is in an off state while powered on may include the steps of: determining if there is a current flowing between an anode and a cathode of the electrotransport drug delivery device when the electrotransport drug delivery device is in an off state while powered on, wherein the electrotransport drug delivery device includes an activation circuit that is configured to apply current between the anode and the cathode to deliver a drug when the device is in an on state and not in the off state; and triggering an indicator if there is a current flowing between the anode and cathode that is greater than an Output Current Off Threshold when the electrotransport drug delivery device is in an off state while powered on. The method may also include repeating the determining step periodically while the activation circuit is in an off state. In some variations the method also includes repeating the determining step at least once every 10 minutes while the activation circuit is in an off state and the device is powered on.

As mentioned above, any appropriate Output Current Off Threshold may be used. For example, an Output Current Off Threshold may be about 9 μA. The step of determining if there is a current flowing between the anode and cathode of the electrotransport drug delivery device may include independently determining an anode voltage and a cathode voltage and comparing the potential difference between the anode voltage and cathode voltage to the threshold value. Any appropriate threshold (e.g., above noise) value may be used. For example, a threshold value may be about 2.5 V. In some variations the threshold value is about 0.85 V.

In some variations, the step of determining if there is a current flowing between the anode and cathode of the electrotransport drug delivery device may include independently connecting a reference voltage source and a sense resistor with each of the anode and cathode to determine the potential difference between the anode voltage and cathode voltage.

Any of the methods described herein may also include activating the activation circuit to enter the on state and applying current between the anode and the cathode after determining that no current above the Output Current Off Threshold is flowing between the anode and cathode while the electrotransport drug delivery device is in the off state.

In any of the devices, systems and methods described herein, the electrotransport device may trigger an indicator and/or modify the state of the device when a current is detected or inferred, between the anode and cathode while the device is in the off state. For example, in some variations, the device may trigger an indicator comprising a visible, audible and/or tactile alert or alarm. For example, an indicator may include illuminating a light and/or sounding an alarm on the device. In some variations, the system may transmit (e.g., electronically, wirelessly, etc.) a signal to another device such as a computer, handheld device, server, and/or monitoring station indicating the alarm status of the device.

In any of the variations described herein, the device, system or method may be configured so that when the off-current module senses or infers a current is flowing between the anode and cathode while the device is in the off state (e.g., while the device is otherwise powered on), the triggering of an indicator may include switching the device to an end of life state, e.g., such as performing a device shutdown. Thus, when the off-current module determines or infers that current is flowing between the anode and cathode when the device is not supposed to be delivering drug, the device (e.g., the off-current module) may prevent further unwanted drug delivery.

In general, when the device or system (or methods of operating them) is described as detecting current flowing between the anode and cathode of the device when the device is not supposed to be delivering drug (e.g., when an off-current module detects current flow between the anode and cathode in an off state) this may be interpreted in some variations as determining if there is a current above some threshold flowing between the anode and cathode. As described above, depending on the way in which the off-current module detects or infers current flow between the anode and cathode, this threshold may be a current threshold, a potential difference (i.e., voltage) threshold, an inductive threshold, a capacitive threshold, or the like. The threshold may be predetermined (preset) in the device.

Also described herein are devices and methods for controlling the application of current and/or voltage to deliver drug from patient contacts of an electrotransport drug delivery device by indirectly controlling and/or monitoring the applied current without directly measuring from the cathode of the patient terminal. In particular, described herein are electrotransport drug delivery systems including constant current delivery systems having a feedback current and/or voltage control module that is isolated from the patient contacts (e.g., anodes and cathodes). In some variations the feedback module is isolated by a transistor from the patient contacts; feedback current and/or voltage control measurements are performed at the transistor rather than at the patient contact (e.g., cathode).

For example, described herein are electrotransport drug delivery systems having a constant current supply. In some variations the system include: a power source; a first patient contact connected to a power source; a second patient contact connected to a current control transistor; and a sensing circuit for measuring voltage at the transistor, wherein the second patient contact is connected to the sensing circuit only through the current control transistor so that the second patient contact is electrically isolated from the sensing circuit. In some variations, the first patient contact may also be connected indirectly to the power source.

The current control transistor may be controlled by an amplifier receiving input from a microcontroller. Any appropriate transistor may be used. For example, the transistor may be a FET or a bipolar transistor. In variations in which the current control transistor is a FET, the second patient contact may be connected to the drain of the transistor.

In some variations, the sensing circuit is configured to compare the voltage at the transistor to a threshold voltage. The sensing circuit may provide input to a feedback circuit. In some variations, this feedback circuit may provide an alarm based on the comparison between the voltage at the transistor (e.g., at the gate of the transistor when the drain is patient-contacting) and the threshold voltage to indicate constant current cannot be maintained. The feedback circuit may automatically control the power source based on the comparison between the voltage at the transistor and the threshold voltage to maintain constant current while minimizing power consumption. For example, in some variations, the current may be maintained at about 170 μA.

Also described herein are electrotransport drug delivery systems having a constant current supply, the system comprising: a power source; a first patient contact connected to the power source; a second patient contact connected to a transistor (e.g., a drain of a transistor); a current control feedback circuit for providing a control signal to the transistor when the connection between the first patient contact and the second patient contact is closed; wherein the transistor is connected to the second patient contact; and a sensing circuit for measuring a voltage applied at the transistor when the connection is closed; wherein the second patient contact is connected to the current control feedback circuit and sensing circuit only though the transistor. For example, the second patient contact may be connected to the drain of the transistor, which is separate from the feedback/sensing circuit that may be connected to the gate of the transistor.

As mentioned above, the transistor may be any appropriate transistor, including a bipolar transistor and/or a field-effect transistor (FET). For example, if the transistor is a FET, the second patient contact may be connected to a drain of the transistor, and the control signal may comprise a voltage applied to a gate of the transistor. In some variations the transistor is a bipolar transistor, and the second patient contact is connected to a collector, while the control signal comprises a current applied to the base of the bipolar transistor. In general, the control signal may be a voltage and/or a current applied to the transistor.

In some variations, the control signal provided to the transistor may be controlled by an amplifier receiving input from a microcontroller.

The feedback circuit may control the voltage applied to the power source. For example, in some variations, the feedback circuit compares the transistor (e.g., gate) voltage to a reference voltage. The feedback circuit controls the power source based on the comparison between the transistor gate voltage and the reference voltage. The feedback circuit may provide a power source sufficient to deliver a constant current. For example, the feedback circuit may provide a power source sufficient to deliver a constant current of about 170 μA. The feedback circuit may include a digital to analogue converter for providing a constant current.

In general, the sensing circuit may be isolated (e.g., electrically isolated) from the first and second patient contacts by the transistor. The transistor may be located between the second patient contact and a sense resistor.

The first patient contact may be an anode and the second patient contact may be a cathode. The connection between the first patient contact and the second patient contact is typically configured to be closed (e.g., connected) by a patient's skin.

Also described herein are methods for operating an electrotransport drug delivery system including a constant current supply, the method comprising: contacting a patient's skin with an anode and cathode to form a connection between the anode and cathode; applying an anode voltage to the anode; providing a control signal to a transistor (e.g., gate) connected to the cathode (e.g., at the drain); detecting a voltage at the transistor, wherein the cathode is isolated from the voltage detection by the transistor; comparing the transistor voltage to a threshold voltage; and controlling the anode voltage applied to the anode based on the comparison between the transistor voltage and the threshold voltage.

The methods may include the use of any appropriate transistor. For example, the transistor may be a FET and the control signal comprises a voltage applied to a gate of the transistor. The anode voltage may be applied to the anode in response to an input. The control signal applied to the transistor may be provided to the transistor by an amplifier, the amplifier isolated from the anode and the cathode by the transistor. As mentioned above, any appropriate control signal may be used, in particular an electrical voltage and/or a current.

In any of these variations, the current provided from the transistor is a constant current. For example, the provided current may be controlled to be about 170 μA.

In some variations, the method includes adjusting the voltage applied to the anode based on the comparison of the transistor voltage to the threshold voltage.

### INCORPORATION BY REFERENCE

All publications and patent applications mentioned in this specification are herein incorporated by reference in their entirety to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings, in which similar features may be identified with the same numbers, of which:

FIG. 1 illustrates an exemplary therapeutic agent delivery system;

FIG. 2 shows an embodiment of iontophoretic therapeutic agent delivery mechanism;

FIG. 3 shows an exemplary embodiment of a controller as connected to an activation switch;

FIG. 4 shows exemplary timing of an activation sequence;

FIG. 5 is an exemplary embodiment of a therapeutic agent delivery device having switch integrity testing;

FIG. 6 is an exemplary embodiment of a therapeutic agent delivery device with switch integrity testing;

FIG. 7 shows exemplary timing of an activation sequence with switch integrity testing;

FIG. 8 shows an equivalent circuit configuration of therapeutic agent delivery device 500 during a short interval switch grounding integrity test;

FIG. 9 shows signaling during the short interval switch grounding integrity test;

FIG. 10 shows an equivalent circuit configuration of therapeutic agent delivery device 500 during a short interval power switch integrity test;

FIG. 11 shows signaling during the short interval power switch integrity test;

FIG. 12 shows an equivalent circuit configuration of therapeutic agent delivery device 500 during a long interval analog switch grounding integrity test;

FIG. 13 shows signaling during the long interval analog switch grounding integrity test;

FIG. 14 shows an equivalent circuit configuration of therapeutic agent delivery device 500 during a long interval analog power switch integrity test;

FIG. 15 shows signaling during the long interval analog power switch integrity test;

FIG. 16 shows a flow chart of the dosing operation of an embodiment of a therapeutic agent delivery device with switch integrity testing; and

FIG. 17 shows an exemplary embodiment of a switch integrity testing process.

FIG. 18A shows a schematic illustration of one variation of a switch and control circuitry for performing both digital and analog validation.

FIG. 18B is a table describing connections of the nodes from the example in FIG. 18A.

FIGS. 19A, 19B and 19C illustrates variations of the timing of dose switch activation sequences for an apparatus or method in which both analog and digital switch validation is performed within the predetermined time period immediately following a second manual switch actuation. FIGS. 19A, 19B and 19C show analog switch validation followed by digital switch validation, digital switch validation followed by analog validation and concurrent analog and digital switch validation, respectively.

FIG. 20 illustrates an exemplary therapeutic agent delivery system in two parts.

FIG. 21 shows the exemplary system of FIG. 20 combined to form a single, unitary device.

FIG. 22 shows an exploded perspective view of a two-part device.

FIG. 23 shows an exploded perspective view of an exemplary reservoir module.

FIG. 24 is a cross-section perspective view of a reservoir contact.

FIG. 25 shows a bottom view of an electrical module and a top view of a reservoir module.

FIGS. 26A and 26B show cross-section views of a power-on connector when open (prior to actuation) and closed by a power-on post acting through a power-on receptacle.

FIG. 27 shows a cross-section view of an output from the electrical module making contact with an input connector on the reservoir module.

FIG. 28 is a circuit diagram for electronics within an electrical module of the device described herein.

FIG. 29 is a flow chart showing a power-on sequence of a device as described herein.

FIG. 30 is a second flow chart showing an alternative power-on sequence of a device as described herein.

FIG. 31A is a block diagram of an exemplary potential difference detection system including a controller, an electrotransport drug delivery circuit, a sensing circuit, an anode and a cathode.

FIG. 31B is a flow diagram of a method of an exemplary automated self-test of an electrotransport drug delivery system configured as an off-current (or anode/cathode voltage difference) test.

FIG. 32A illustrates an exemplary therapeutic agent delivery system in two parts as already shown in FIG. 20.

FIG. 32B shows the exemplary system of FIG. 31A combined to form a single, unitary device.

FIG. 33 shows an exploded perspective view of a two-part device.

FIG. 34 shows an exploded perspective view of an exemplary reservoir module.

FIG. 35 is a cross-section perspective view of a reservoir contact.

FIG. 36 shows a bottom view of an electrical module and a top view of a reservoir module.

FIGS. 37A and 37B show cross-section views of a power-on connector when open (prior to actuation) and closed by a power-on post acting through a power-on receptacle.

FIG. 38 shows a cross-section view of an output from the electrical module making contact with an input connector on the reservoir module.

FIG. 39 is a circuit diagram for electronics within an electrical module of the device described herein.

FIG. 40 is a flow chart showing a power-on sequence of a device as described herein.

FIG. 41 is a second flow chart showing an alternative power-on sequence of a device as described herein.

FIG. 42 is a diagram showing the user mode diagram for one exemplary embodiment of a system including an off-current self-test module.

FIG. 43 shows an example of a software block diagram for the example of FIG. 42.

FIG. 44 illustrates one variation a procedure for system initialization.

FIG. 45 shows a software state chart for the Example of FIG. 42.

FIG. 46 is an exemplary diagram of a current control circuit for one variation of a drug delivery device.

FIG. 47 shows a Dosing Mode Flow Diagram.

FIG. 48 shows a Dose Initiation Flow Diagram.

FIG. 49 shows a Dose Control Flow Diagram.

FIG. 50 shows a Dose Completion Flow Diagram.

FIG. 51 shows Table 1, indicating one variation of the sequencing of self-testing (the mode diagram of FIG. 42 may correspond with this table).

FIG. 52 is a schematic of a prior art iontophoretic transdermal drug delivery system.

FIG. 53 is a block diagram of an exemplary electrotransport drug delivery circuit for use with an electrotransport drug delivery system including a controller, a drug delivery circuit, a feedback circuit, an anode and a cathode.

FIG. 54 is a schematic diagram of the feedback circuit of FIG. 53.

FIG. 55 is a schematic diagram of the electrotransport drug delivery circuit of FIG. 53.

FIG. 56 is a flow diagram of a method of operation of an exemplary electrotransport drug delivery circuit.

### DETAILED DESCRIPTION

Generally described herein are iontophortic drug delivery apparatuses (e.g., systems and devices) and methods of using them. In particular, described herein are fault-resistant and/or fault-detecting apparatuses. Also described herein are two-part electrotransport therapeutic agent delivery device, such as an iontophoresis device, in which the two parts of the device are provided separately and assembled to form a unitary, powered-on device at the point of use—that is to say just prior to use. The apparatuses described herein permit the combination of the electrical module and the reservoir module, whereby in a single action the two modules form a single unit and the battery is introduced into the circuitry, thereby powering on the device, in a single action by the user. Also described herein are systems and devices that include an anode and cathode for the electrotransport of a drug or drugs into the patient (e.g., through the skin or other membrane) and a controller for controlling the delivery (e.g., turning the delivery on or off); any of these apparatuses may also include an off-current module for monitoring the anode and cathode when the activation circuit is in the off state while still powered on to determine if there is a potential and/or current (above a threshold value) between the anode and cathode when the controller for device has otherwise turned the device “off” so that it should not be delivering drug to the patient. Also described herein are devices that include control logic and/or circuitry for regulating the application of current by the device. For example, a feedback circuit may be controlled or regulated by a controller and be part of (or separate from) the drug delivery circuit. The controller and circuit may include hardware, software, firmware, or some combination thereof (including control logic). Any of the variations or features (including portions, subcombinations and combinations thereof) may be be combined with any of the other variations or features described herein.

For example, as just mentioned, described herein provide circuitry and methods for actively detecting faults and precursors to faults in devices, such as drug delivery devices, and more particularly iontophoretic drug delivery devices.

In some embodiments, there is provided a switch operated device, such as a drug delivery device (e.g. a drug delivery pump, electrotransport device or iontophoresis device). The device comprises (a) a device switch configured to be operated by a user, which provides a switch signal to a switch input of a device controller when operated by a user; (b) the device controller, having said switch input operatively connected to the switch, and configured to receive the switch signal from the switch, the device controller being configured to actuate the device when the switch signal meets certain predetermined conditions; and (c) a switch integrity test subcircuit, which is configured to detect a fault or a precursor to a fault in the switch, whereby the controller executes a switch fault subroutine when a fault or a precursor to a fault is detected. When the device is an iontophoretic drug delivery device, the device further comprises other circuitry components, such as electrodes, one or more drug also called active reservoirs and one or more counter ion reservoirs which are capable of delivering drug to a patent in response to patient input. An iontophoretic drug delivery device (iontophoresis devices) is illustrated below, though iontophoresis is well-characterized and is described in detail in U.S. Pat. No. 7,027,859, for example.

In some embodiments, the switch integrity test subcircuit is configured to check for and detect a fault or a precursor to a fault in the switch or connecting circuitry. In some preferred embodiments, the act of checking for a fault or precursor to a fault includes setting a circuit condition to evoke a response in the circuit (for example, change in voltage, change in current) which is expected to fall within predetermined parameters if the circuit and its components are free of faults or precursors to faults. In some embodiments, the switch integrity test subcircuit is configured to test for and detect at least one fault or precursor to a fault, such as a member of the group selected from the group consisting of contamination, shorts, (including intermittent short circuits), compromised circuit components (including malfunctioning resistors, integrated circuit pins or interfaces, and/or capacitors), etc. Among the advantages of the device and methods described herein, there may be mentioned the ability to detect and respond to precursors to faults before they manifest in such a manner as to cause the device to malfunction in a way to compromise patient comfort, safety and/or compliance. This aspect of device and methods is described in more detail herein, but includes the ability to actively test for and detect subtle deviations in circuit characteristics from predetermined normal circuit characteristics.

In some embodiments, the switch integrity test subcircuit is configured to test for and detect a voltage or change in voltage in between a short between the switch input and ground or some intermediate voltage above ground (low voltage, V_{L}), a short between the switch input and a voltage pull up or some intermediate voltage below a pull up voltage (high voltage, V_{H}). In some preferred embodiments, the switch integrity test subcircuit is configured to test for and detect a voltage or change in voltage in between a short between the switch input and some intermediate voltage above ground (low voltage, V_{L}) and/or a short between the switch input and intermediate voltage below a pull up voltage (high voltage, V_{H}) Thus, the switch integrity test subcircuit is configured to test for and detect a damaged circuit resistor, contamination (e.g., humidity, particulates), corrosion and/or a damaged integrated circuit pin or integrated circuit interfaces, etc. In particular embodiments, the switch integrity test subcircuit includes the controller and additional circuit components under control of the controller, which the controller is capable of placing in certain states to cause certain effects in the circuit. By detecting the effects that arise when the controller places the circuit components in those predetermined states, and comparing the effects to those which are considered normal for the device, the controller can detect faults and precursors to faults in the device circuitry. It is a particular advantage of the instant device and methods that precursors to faults may be detected before they have manifested in such a way that their effects would be experienced by a patient.

When the switch integrity test subcircuit detects a fault or a precursor to a fault, it provides a fault signal to the controller, which in turn executes a switch fault subroutine, which includes, for example, at least one of: activating a user alert feature, logging detection of faults or precursors to faults, deactivating the device, or one or more combinations thereof. The user alert feature can include a variety of means to alert a user that operation of the system is considered compromised. Since the device is configured, in some embodiments, to detect precursors to faults, the device may activate the user alert even before a fault has been detected that would cause an effect that would be experienced by the patient. The user alert may be an indicator light, such as a colored light emitting diode (LED), an audible tone (such as a repeating “beep”), a readable display (such as a liquid crystal display (LCD)), other user observable indicator (such as a text message, email, voicemail, or other electronic message sent to a device that is observable by the patient, the caregiver or both), or combinations of two or more thereof.

As used herein, unless otherwise defined or limited, the term “when” indicates that a subsequent event occurs at the same time as or at some time after a predicate event. For the sake of clarity, “switch integrity test subcircuit detects a fault or a precursor to a fault, it provides a fault signal to the controller, which in turn executes a switch fault subroutine . . . ” is intended to indicate that the subsequent act of executing the switch fault subroutine happens as a consequence of (e.g., at the time of, or at some time after) the predicate event of detection of the fault or precursor to the fault. The term “when” is intended to have analogous effect throughout this disclosure unless otherwise indicated.

In some embodiments, the controller can also log detection of faults or precursors to faults in memory, such as flash memory. In some such embodiments, the controller detects a certain type of fault, assigns it a fault code, and records the fault code in memory for retrieval at a later time. For instance, the controller may detect and record one of the following conditions: a low voltage at a point and under conditions where a high voltage would be expected for a normally operating circuit; a voltage at a point and under conditions that is higher or lower than the voltage that would be expected for a normally operating circuit; a voltage rise time that is longer or shorter than would be expected for a normally operating circuit; a voltage fall time that is longer or shorter than would be expected for a normally operating circuit; or combinations of two or more thereof.

In some embodiments, the switch fault subroutine includes deactivating the device. Methods of deactivating a device, e.g. by irreversibly decoupling the voltage supply from the drug delivery circuit, shorting a power cell to ground, fusing a fusible link in the circuit, etc., are known. In some embodiments, the circuitry and methods employed in U.S. Pat. No. 7,027,859, which incorporated herein by reference, especially those recited between line 65 of column 6 and line 12 of column 8 of U.S. Pat. No. 7,027,859 (and the accompanying figures) may be adapted to disable the circuit when the controller detects a voltage or current, or change thereof, that is outside of predetermined parameters.

In some preferred embodiments, devices and methods taught herein will be capable of performing two or more of the functions of activating a user alert feature (e.g. activating a light and/or audible sound), logging the detected fault or precursor to a fault, and/or deactivating a device. In some preferred embodiments, the devices and methods taught herein are capable of activating a user alert feature, deactivating the device and optionally logging the detected fault or precursor to a fault.

In some embodiments, the controller is configured to measure a voltage or a rate of change of voltage at the switch input and execute the switch fault subroutine when the voltage or rate of change of voltage at the switch input fails to meet one or more predetermined parameters. In some embodiments, the device is an iontophoresis delivery device comprising first and second electrodes and reservoirs, at least one of the reservoirs containing therapeutic agent to be delivered by iontophoresis. It is to be understood that the terms “higher” and “lower” are relative. Especially in embodiments in which the device is capable of detecting and responding to precursors to faults, the terms “higher” and “lower” may express deviations of as little as 10%, 5%, 2% or 1% of the expected values. For example, in terms of voltages, a voltage that is higher than expected may be greater than from 10-200 mV, 10-100 mV, 10-50 mV, 20-200 mV, 20-100 mV, 20-50 mV, 50-200 mV, 50-100 mV, or 100-200 mV higher than the nominal voltage expected at the point and under the conditions tested. In particular, the “higher” voltage may be greater than 10 mV, 20 mV, 50 mV, 75 mV, 100 mV, 125 mV, 150 mV, 175 mV, 200 mV or 250 mV than would be expected at the same point under the conditions tested. Also in terms of voltages, a voltage that is lower than expected may be at least from 10-200 mV, 10-100 mV, 10-50 mV, 20-200 mV, 20-100 mV, 20-50 mV, 50-200 mV, 50-100 mV, or 100-200 mV lower than the voltage expected at the point and under the conditions tested. In particular, the “lower” voltage may be at least 10 mV, 20 mV, 50 mV, 75 mV, 100 mV, 125 mV, 150 mV, 175 mV, 200 mV or 250 mV less than would be expected at the same point under the conditions tested. Voltage rise and fall times may be characterized in the amount of time necessary (e.g., measured in ms or μs) for a point under a condition tested to achieve an expected voltage state. In terms of rise or fall times, the difference in rise or fall time from the expected rise or fall time may be as little as 1 ms or as much as 20 ms, e.g. 1, 2, 5, 10, 12.5, 15 or 20 ms, depending upon the point tested under the particular conditions. Voltage and current rise times may also be characterized by measuring a change in voltage or current between two selected time points and comparing them to the change in voltage or current that would be expected for a normally operating circuit at the point and under the condition tested.

In some preferred embodiments, the device is capable of detecting subtle differences in circuit states—whether voltages, currents, changes in voltages or changes in currents. These subtle changes may indicate that the circuit board has been contaminated with one or more contaminants, is experiencing intermittent shorts between circuit components, has one or more compromised circuit components, or combinations thereof. Such embodiments permit the device to identify precursors to faults before they manifest as circuit faults that can affect delivery of a drug and in particular before they are noticed by, or affect, a patient.

In some embodiments, the predetermined conditions for actuating the device include the user activating the switch at least two times within a predetermined period of time. This feature permits the device to distinguish between purposeful activation of the switch by a user (patient or caregiver, preferably a patient) and spurious or accidental button pushes, e.g. those that occur during shipping or storage, those that occur from contamination, or those that may accidentally occur during placement of the device on the patient or during movement of the patient after the device has been applied to the patient. Activation of the switch by multiple button pushes or the like is described with reference to the figures herein. The time between button pushes—which is typically on the order of at least a few hundred milliseconds (ms)—affords one time window during which the device controller can actively test the switch circuit. In some embodiments, the device is configured such that the device will initiate drug delivery when it receives two distinct button pushes of a predetermined separation in time—e.g. on the order of 100-400 ms, preferably about 300 ms. During this period, which may be referred to as the test period, the controller can actively set certain circuit parameters (using the switch integrity test subcircuit), test voltages or changes in voltages at certain points and compare them to predetermined values that are indicative of what a normally operating circuit—i.e. a circuit that is not manifesting a fault or a precursor to a fault—would manifest. For example, the controller may set a switch input to a low state and remove a high supply voltage (V_{DD}), then check whether the switch input achieves a true low (expected) of 0 mV above the low supply voltage (V_{SS}, e.g., ground or some voltage above ground), or if it fails to achieve such a true low (indicating a fault or precursor to a fault) of at least 5 mV to at least 250 mV above V_{SS} (e.g. at least 5, 10, 15, 20, 25, 30, 35, 40, 45, 50, 75, 100, 125, 150, 200, 225 or 250 mV above V_{SS}). If a fault or precursor to a fault is detected, the device controller will then initiate a switch fault subroutine, as described elsewhere herein.

As used herein, V_{DD} refers to any predetermined high voltage (V_{D}), and need not be the highest voltage available from the power supply. Likewise, V_{SS} refers to any predetermined low voltage (V_{L}) and need not indicate “ground”. Among other advantages, one advantage of the device and method described herein is that intermediate voltages may be used to test switch integrity, which allows for detection of spurious voltages that indicate contaminants (e.g. humidity, particulates, corrosion, etc.) and other faults and precursors to faults. The precise values of V_{DD} and V_{SS} are selected by the artisan during device design.

In other exemplary embodiments, for example, the controller may set a switch input to a V_{DD} (e.g. a value of from 2 V to 15 V, such as 5 V or 10 V) and connect the switch input to V_{SS} (e.g. a value of 0 V to 1 V above ground), then check whether the switch input achieves V_{DD} (as expected), or if it fails to achieve V_{DD} (indicating a fault or precursor to a fault) by at least 5 mV to at least 250 mV lower than V_{DD} (e.g. at least 5, 10, 15, 20, 25, 30, 35, 40, 45, 50, 75, 100, 125, 150, 200, 225 or 250 mV lower than V_{DD}).

In some embodiments, the switch input is pulled up to V_{DD} when the switch is open and the switch input is V_{SS} when the switch is closed. Other configurations are possible. For example, with a change in the logic of the controller, the switch input could be biased to V_{SS}, meaning that upon a button push the switch input would be pulled high. The person of skill in the art will recognize that other configurations, including those requiring three, four or more sequential button pushes may be employed, though in general the inventors consider two to be sufficient for most purposes.

Some embodiments described herein provide a method of switch fault detection in a switch operated device, said device comprising: (a) a device switch connected to a switch input of a device controller; (b) the device controller comprising said switch input; and (c) a switch integrity test subcircuit, said method comprising said controller: (i) activating the switch integrity test subcircuit; (ii) detecting a voltage condition at the switch input; and (iii) activating a switch fault subroutine if the voltage condition at the switch input fails to meet one or more predetermined conditions. These methods may be carried out using for example those circuits and devices described herein.

In some embodiments, the steps of activating the switch integrity test subcircuit and detecting a voltage condition at the switch input are executed continuously or periodically throughout operation of the device. Without limitation, such a method may include digital or analog testing. Digital testing is relatively fast and is well-suited to performance during the test period between button pushes. Analog testing may be either fast or slow, depending upon how many data points are collected. Analog testing may be, and in some embodiments is, more sensitive and is well-adapted for detection of very subtle deviations from expected device parameters which are symptomatic of precursors to faults. Fast analog testing is well-suited for detection after any button bounce or anything (any voltage signal) that looks like (could be interpreted by the controller as) a button push. Analog testing is also well-suited for the period when drug is being delivered to a patient (that is after the second button press in the case where the device is activated by two distinct button presses) or even during the period between drug delivery intervals (that is when the device is still attached to the patient but is not currently delivering drug). In the latter case, the device may administer a very small amount of current for a brief period of time (e.g. 500 ms to 10 seconds, more preferably 500 ms to 5 seconds, even more preferably 500 ms to 1 second) during which time the controller carries out its active checking. As described herein, analog checking, whether between button pushes, during the dosing period or between dosing periods, is very sensitive and may detect subtle changes in circuit properties before they develop into full-fledged faults, thus permitting avoidance of untoward events before they can manifest. In some embodiments, testing may include a combination of digital and analog testing. In some preferred embodiments, a fast analog test is conducted after any button push (including detection by the controller of any voltage signal that it interprets as a button push) and/or a digital test is conducted after a second button push. In some preferred embodiments, a fast analog test is conducted after any button push (including detection by the controller of any voltage signal that it interprets as a button push) and a digital test is conducted after a second button push. In some embodiments, a slow analog test is conducted in addition to the digital test sometime after the second button push.

Some embodiments described herein provide a switch operated iontophoresis therapeutic agent delivery device, comprising: (a) a power source; (b) first and second electrodes and reservoirs, at least one of the reservoirs containing the therapeutic agent; (c) a device switch, which provides a switch signal to a switch input of a device controller when operated by a user; the device controller having said switch input operatively connected to the switch, whereby the controller receives the switch signal from the switch, the device controller being operatively connected to a power source that provides power to the first and second electrodes for delivering therapeutic agent to a patient; and (d) a switch integrity test subcircuit, which is configured to detect a fault in the switch and cause the controller to execute a switch fault subroutine when a fault is detected. In some embodiments, the therapeutic agent is fentanyl or sufentanil. For the sake of clarity, “fentanyl” includes pharmaceutically acceptable salts of fentanyl, such as fentanyl hydrochloride and “sufentanil” includes pharmaceutically acceptable salts of sufentanil.

Some embodiments described herein provide a method of switch fault detection in a user operated iontophoresis therapeutic agent delivery device, said device comprising: (a) a power source; (b) first and second electrodes and reservoirs, at least one of the reservoirs containing the therapeutic agent; (c) a device switch connected to a switch input of a device controller; (d) the device controller comprising said switch input and configured to control power to the first and second electrodes, thereby controlling delivery of the therapeutic agent; and (e) a switch integrity test subcircuit, said method comprising said controller: (i) activating the switch integrity test subcircuit; detecting a voltage condition at the switch input; and (ii) activating a switch fault subroutine if the voltage condition at the switch input fails to meet one or more predetermined conditions. In some embodiments, the switch fault subroutine includes activating a user alert, deactivating the device, or both.

The present invention relates generally to apparatus (e.g., electrical circuits) which are used to enhance the safety of electrophoretic drug delivery. Drugs having particular potential for use iontophoretic drug delivery include natural and synthetic narcotics. Representative of such substances are, without limitation, analgesic agents such as fentanyl, sufentanil, carfentanil, lofentanil, alfentanil, hydromorphone, oxycodone, propoxyphene, pentazocine, methadone, tilidine, butorphanol, buprenorphine, levorphanol, codeine, oxymorphone, meperidine, dihydrocodeinone and cocaine. In the context of iontophoresis, it is to be understood that when reference is made to a drug, unless otherwise stated, it is intended to include all pharmaceutically acceptable salts of the drug substance. For example, where reference is made to fentanyl, the inventors intend that term to include fentanyl salts that are suitable for delivery by iontophoresis, such as fentanyl hydrochloride. Other exemplary pharmaceutically acceptable salts will be apparent to the person having ordinary skill in the art.

For the sake of clarity, as used herein, the terms “therapeutic agent” and “drug” are used synonymously, and include both approved drugs and agents which, when administered to a subject, are expected to elicit a therapeutically beneficial effect. For the sake of further clarity, where a particular drug or therapeutic agent is recited, it is intended that that recitation include the therapeutically effective salts of those therapeutic agents.

Reference is now made to the figures, which illustrate particular exemplary embodiments of the device and methods taught herein. The person having skill in the art will recognize that modifications and various arrangements of the illustrated circuits and methods are within the scope of the instant disclosure and claims.

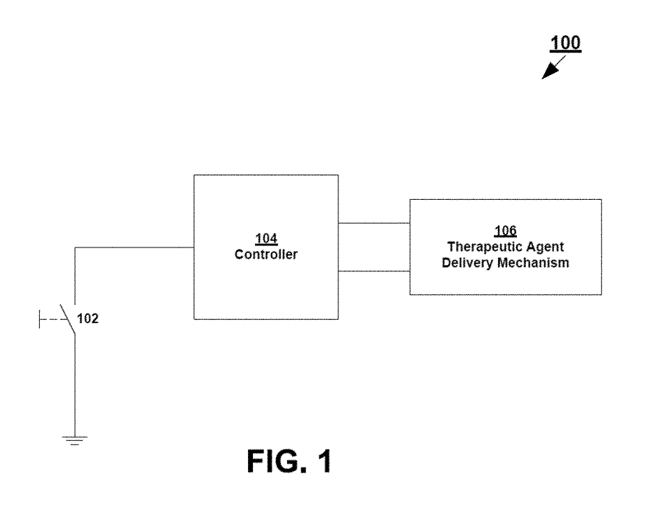

FIG. 1 illustrates an exemplary therapeutic agent delivery system. Therapeutic agent delivery system 100 comprises activation switch 102, controller 104 and therapeutic agent delivery mechanism 106. Activation switch 102 can be selected from a variety of switch types, such as push buttons switch, slide switches and rocker switches. In some embodiments, a push button switch is used. Though either a “momentary on” or “momentary off” push button switch can be used, for the sake of clarity, a momentary on push button switch is given in each example. Controller 104 controls the administration of drugs to the patient as to the specific rate and amount a drug is dispensed. It can also be used to regulate the dosing interval. For example, for a pain medication, the controller could allow a patient to receive a dose at most once in a predetermined time period, e.g. once every five minutes, ten minutes, 15 minutes, 20 minutes, one hour or two hours. Controller 104 can also comprise a power source, such as a battery, or can simply regulate a power source external to the controller. Typically, the power source controlled by controller 104 is used to drive the delivery of the therapeutic agent through therapeutic agent delivery mechanism 106. Controller 104 can be implemented in a number of ways known in the art. It can comprise a microprocessor and memory containing instructions. Alternatively, it can comprise an appropriately programmed field-programmable gate array (FPGA). It can be implemented in discreet logic or in an application specific integrated circuit (ASIC).

Therapeutic agent delivery mechanism 106 can be selected from a variety of dosing mechanisms including iontophoresis and IV-line pumps. In the former case, a small electric charge which is controlled by controller 104 is used to deliver a drug through a patient's skin. In the latter case, the controller 104 controls a pump which introduces the drug into an intravenous line. For the sake of clarity, the examples herein refer to an iontophoretic drug dispenser.

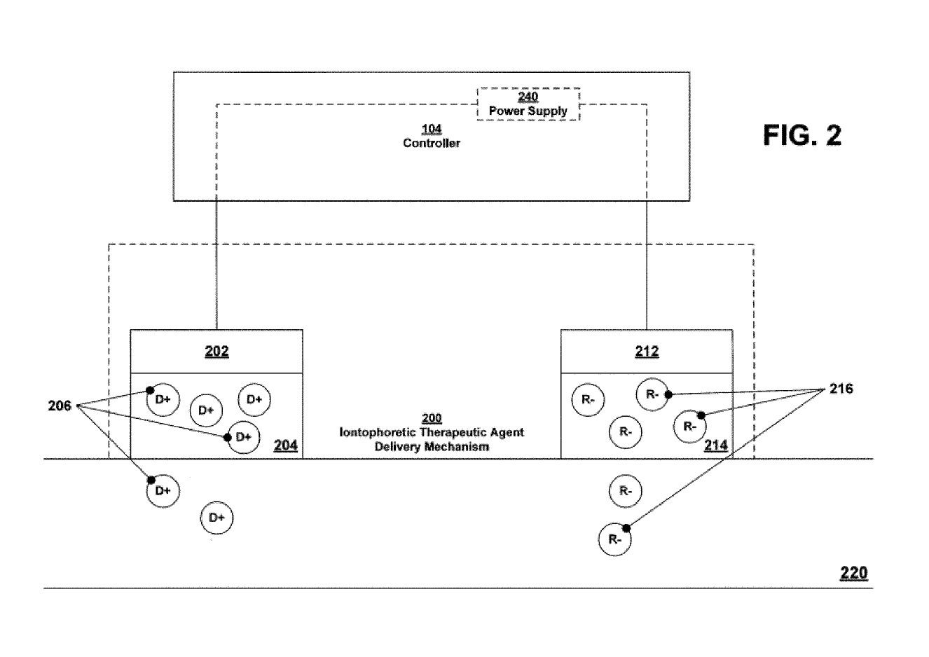

FIG. 2 shows an embodiment of iontophoretic therapeutic agent delivery mechanism. Iontophoretic therapeutic agent delivery mechanism 200 comprises active electrode 202, active reservoir 204, return electrode 212, counter ion reservoir 214. Active electrode 202 and return electrode 212 are electrically coupled to controller 104. Iontophoretic therapeutic delivery agent delivery mechanism 200 often takes the form of a patch which is attached to the skin of a patient (220). Active reservoir 204 contains ionic therapeutic agent 206, which can be a drug, medicament or other therapeutic agent as described herein and has the same polarity as the active electrode. Counter ion reservoir 214 contains counter ion agent 216, which is an ionic agent of the opposite polarity as the ionic therapeutic agent which can be saline or an electrolyte. In other embodiments, iontophoretic therapeutic delivery mechanism 200 can further comprise additional active and/or counter ion reservoirs.

When controller 104 applies a voltage across active electrode 202 and return electrode 212, the patient's body completes a circuit. The electric field generated in this fashion conducts ionic therapeutic agent 206 from active reservoir 204 into the patient. In this example, controller 104 comprises power supply 240 which can be a battery. In other embodiments controller 104 controls an external power source. Therapeutic agent delivery mechanism 200 often comprises a biocompatible material, such as textiles or polymers, which are well known in the art as well as an adhesive for attaching it to a patient's skin.

In some embodiments, controller 104 and iontophoretic therapeutic agent delivery mechanism 200 are assembled together at the time of application of the therapeutic agent. This packaging permits ready application and insures the integrity of the therapeutic agent, but can also introduce addition points of failure of the delivery device.

Therapeutic agent delivery system 100 is often used in circumstances which allow a patient to self-administer drug. For example, an analgesic agent (such as fentanyl or sufentanil, especially in form of a hydrochloride or other deliverable salt) may be self-administered using such a device. In such a circumstance, a patient can self-administer the analgesic agent whenever he feels pain, or whenever the patient's pain exceeds the patient's pain tolerance threshold. Numerous safeguards and safety features are incorporated into controller 104, in order to ensure the patient's safety. In order to ensure proper delivery in an iontophoretic therapeutic agent delivery system, the device may be configured to take into account the varying resistance of the patient's skin among other elements in the circuit. Thus, controller 104 can regulate the amount of current delivered to the patient in order to permit consistent delivery of the therapeutic agent, by monitoring the current (e.g., by measuring the voltage across a current sensing resistor) and adjusting the voltage up or down accordingly. Furthermore, if the condition of the voltage supply prevents proper operation (e.g., weak battery), the device can shut down.

In operation, it is often convenient for the patient who is not acquainted with the particulars of drug application, and who may also be in painful distress, to allow a button press to activate the delivery of the therapeutic agent. Controller 104 upon activation can administer a single dose at the prescribed rate. To prevent inadvertent dosing, controller 104 can require the patient to activate activation switch 102 twice within a predetermined interval. As previously described, a predetermined test period interval can be used to insure that a single switch activation attempt by the patient is not incorrectly interpreted as two switch activation attempts. As described herein, this test period interval provides one convenient period during which a device as described herein can detect and respond to a fault or a precursor to a fault, e.g. using an analog or digital fault checking method.

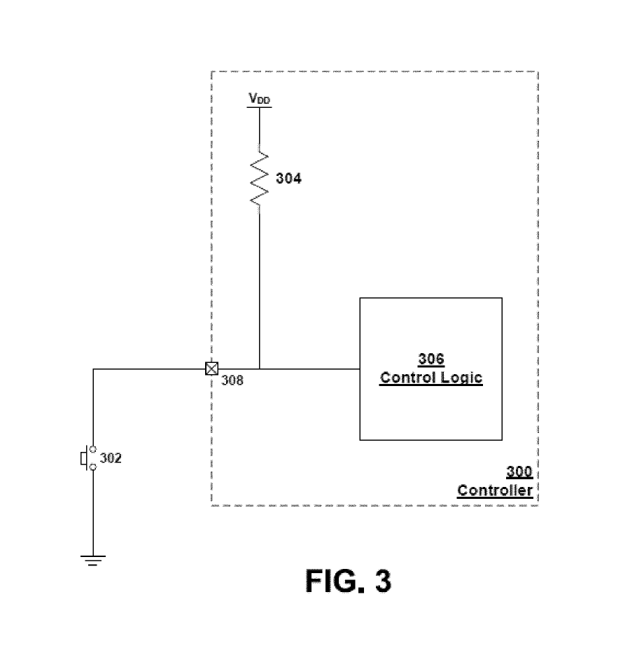

FIG. 3 shows an exemplary embodiment of a controller as connected to an activation switch. Activation switch 302 is shown as a push button momentary “on” switch and is coupled to the ground plane and to controller 300 through switch input 308. Controller 300 comprises pull up resistor 304 and control circuit 306. Pull up resistor 304 is coupled to a supply voltage V_{DD} and switch input 308. Control circuit 306 is also coupled to switch input 308. When activation switch 302 is open, pull up resistor 304 pulls the voltage at switch input 308 to the level of the supply voltage V_{DD}. When the activation switch 302 is closed, it pulls the voltage at switch input 308 down to ground.

Although for the sake of illustration reference is made here to V_{DD}, V_{SS} and “ground” it is to be understood that wherever reference is made to V_{DD}, unless otherwise specified, this is intended to include any predetermined logic level high (V_{H}). Likewise, wherever reference is made to V_{SS} or “ground”, it is intended, unless otherwise specified, to include any predetermined logic level low (V_{L}). In some preferred embodiments, the logic high level is an intermediate voltage below V_{DD} and/or the logic low level is some intermediate voltage above ground. In some preferred embodiments, in fact, the logic high level is an intermediate voltage below V_{DD} and the logic low level is some intermediate voltage above ground. For the sake of clarity, in some places herein the logic high may be referred to as V_{H} and the logic low may be referred to as V_{L}. The use of V_{H} below V_{DD} and/or V_{L} above ground (or V_{SS}) permits the detection of indeterminate voltage signals that arise out of contamination, corrosion or other faults and precursors to faults.

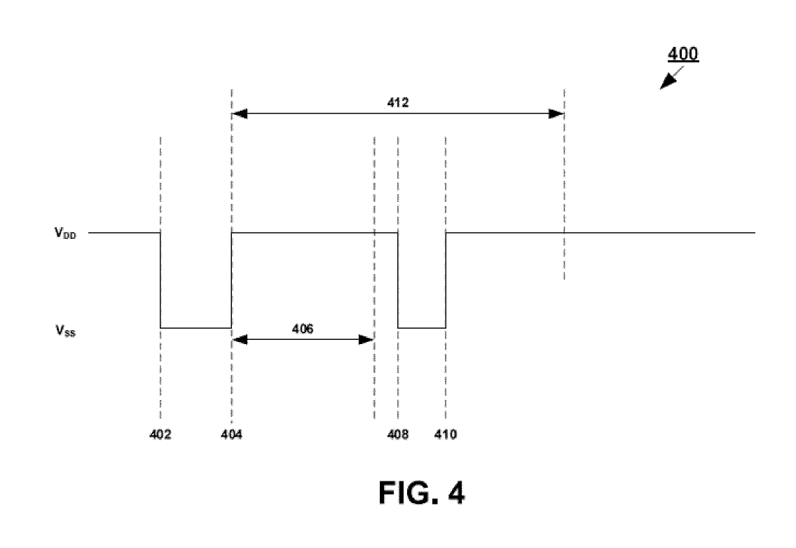

FIG. 4 shows exemplary timing of an activation sequence. Trace 400 shows a plot of voltage at the switch input as a function of time. At time 402, the push button is depressed causing the voltage at switch input 308 to drop to the ground potential. At time 404, the push button is released causing the voltage at switch input 308 to return to the supply voltage level. To further enhance the robustness of the activation of the device, controller 300 enforces a predetermined minimum time interval 406 and a predetermined maximum time interval 412 between the release of the button after the first button press and the second pressing of the button. Should a button press occur before predetermined minimum time interval 406 has elapsed, it is ignored, as during this period it is not clear as to whether a second button press was intended or not. This interval is long enough to avoid an accidental reading, but sufficiently short that an average patient would have a difficult time pressing the button faster than the predetermined minimum time interval. Exemplary predetermined minimum time intervals are given in the overview discussed above. At time 408, which occurs after predetermined minimum time interval has elapsed, a second button press occurs, followed by a button release at time 410. Upon validating the second button press after time 410, controller 300 accepts the sequence as a valid activation sequence and the delivery of the therapeutic agent can begin, provided the second button press is completed before the predetermined maximum time interval has elapsed, for example within 3 seconds. This ensures that an accidental first button press does not leave the therapeutic agent delivery device armed so a second accidental button press could activate the delivery of the therapeutic agent. The activation sequence ensures the therapeutic agent is not delivered accidentally. In addition to ensuring that the therapeutic agent is only delivered when the patient desires it, controller 300 can also incorporate logic and/or circuitry which prevent over-dosing of the therapeutic agent as well as prevent the dispensing of the therapeutic agent after a predetermined lifetime. Such logic and circuitry are described for instance in U.S. Pat. No. 7,027,859, which is incorporated by reference in its entirety, especially as described elsewhere herein. Again, although V_{DD} and V_{SS} are used for illustrative purposes in FIG. 4, any logical high (V_{H}) can be used instead of V_{DD} and any logical low (V_{L}) can be used instead of V_{SS}. In some embodiments V_{H}<V_{DD} or V_{L}>V_{SS}. In some embodiments V_{H}<V_{DD} and V_{L}>V_{SS}.

Additional safeguards to ensure the integrity of the switch can also be implemented into controller 300. For example, controller 300 can detect whether there is a short (including an intermittent short) between switch 302 and either the ground plane or a power supply trace, which can result from contamination or corrosion. The short circuit can be a “hard” short or an intermittent short. Shorts, including intermittent shorts, can be caused by, for example, corrosion or contamination on the circuit. The corrosion or contamination can provide an electrical pathway, which may be continuous or spurious. Additionally, controller 300 can detect whether there is damage to the switch input, which could be an integrated circuit pin or integrated circuit interface pad. A short due to contamination or corrosion, especially an intermittent short, may not necessarily cause the device to malfunction per se. Initially, the contamination or corrosion can manifest itself in a high resistance path between switch 302 and the ground plane or power supply trace; but over time, as the contamination or corrosion accumulates, the resistance of this path may decrease until ultimately the switch may fail. Therefore, the presence of even a high resistance short is indicative of a future fault. Accordingly, in some embodiments, the controller will detect intermittent shorts such as those described and initiate a suitable switch fault subroutine, as described herein. For example, the switch fault subroutine may include setting one or more suitable user alerts (e.g. and audible tone or a visible indicator) and/or disabling the device (e.g. by disconnecting the power supply from the electrodes).

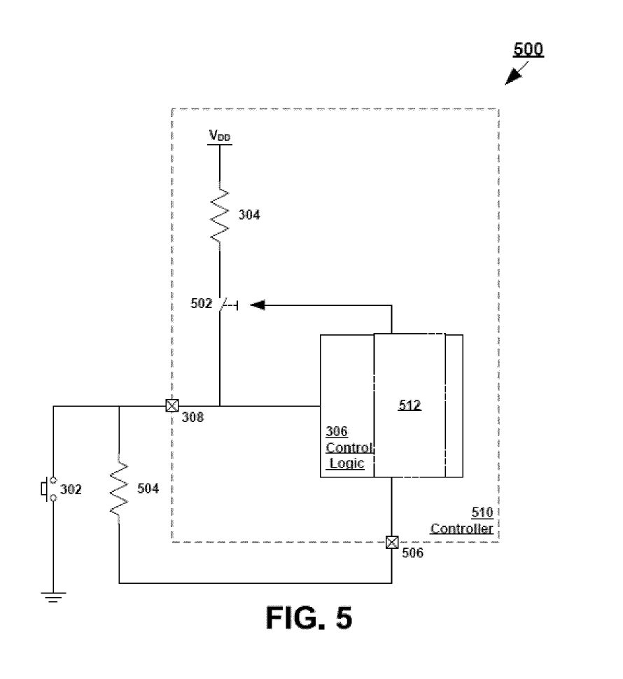

FIG. 5 is an exemplary embodiment of a therapeutic agent delivery device embodying switch integrity testing Like controller 300, controller 510 comprises control logic 306, pull up resistor 304, and switch input 308. Controller 510 further comprises a switch integrity test subcircuit comprising switch 502 (which can be used to electrically decouple pull up resistor 304 from switch input 308), switch integrity test output 506 and integrity test sublogic 512 within control logic 306. Switch integrity test subcircuit is activated when switch integrity testing is performed. Integrity test sublogic 512 is configured to open switch 502 and set switch integrity output 506 to a predetermined voltage or sequence of voltages in accordance with a particular switch integrity test. In an implementation where controller 510 resides on an integrated circuit, switch integrity test output 506 can be implemented with a general purpose I/O port or an analog input pin. Switch integrity test output 506 is coupled to switch input 308 with resistor 504 which generally has a high resistance (e.g., 1 MΩ). Switch integrity test output 506 can be left floating, can provide a high supply voltage (V_{DD}) or can provide a low supply voltage (V_{SS}) (e.g., ground potential). During testing, switch 502 is opened electrically, decoupling pull up resistor 304 from switch input 308. Depending on the desired test, switch integrity test output 506 provides a high supply voltage or a low supply voltage. Greater detail is given in the following description. For clarity integrity test sublogic 512 is omitted from further diagrams. Again, although V_{DD} and ground are used for illustrative purposes in FIG. 5, any logical high (V_{H}) can be used instead of V_{DD} and any logical low (V_{L}) can be used instead of ground. In some embodiments V_{H}<V_{DD} or V_{L}>ground. In some embodiments V_{H}<V_{DD} and V_{L}>ground.

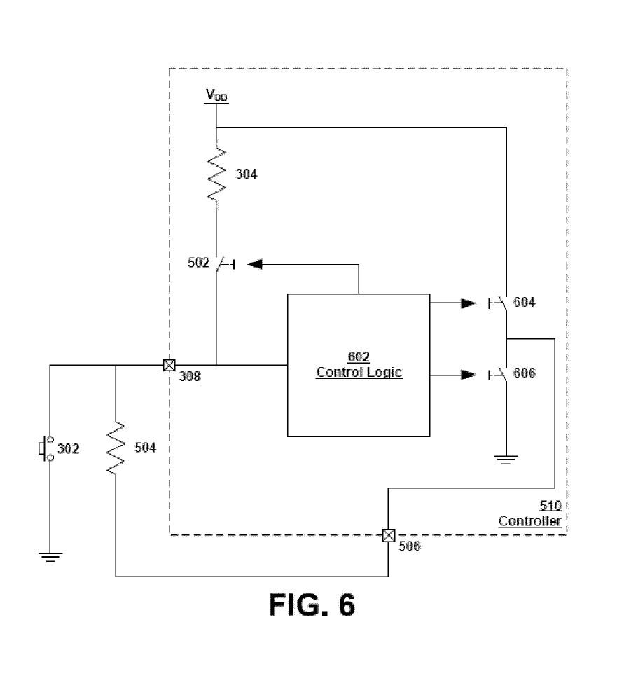

FIG. 6 is an exemplary embodiment of a therapeutic agent delivery device with switch integrity testing. More specifically, controller 510 and more specifically integrity sublogic 512 (not shown) comprises switch 604 and switch 606 which are controlled by control logic 602. When switch 604 and switch 606 are open switch integrity test output 506 is left floating. When switch 604 is closed and switch 606 is open, switch integrity test output 506 provides a high supply voltage. When switch 604 is open and switch 606 is closed, switch integrity test output 506 provides a low supply voltage. Again, although V_{DD} and ground are used for illustrative purposes in FIG. 6, any logical high (V_{H}) can be used instead of V_{DD} and any logical low (V_{L}) can be used instead of ground. In some embodiments V_{H}<V_{DD} or V_{L}>ground. In some embodiments V_{H}<V_{DD} and V_{L}>ground.

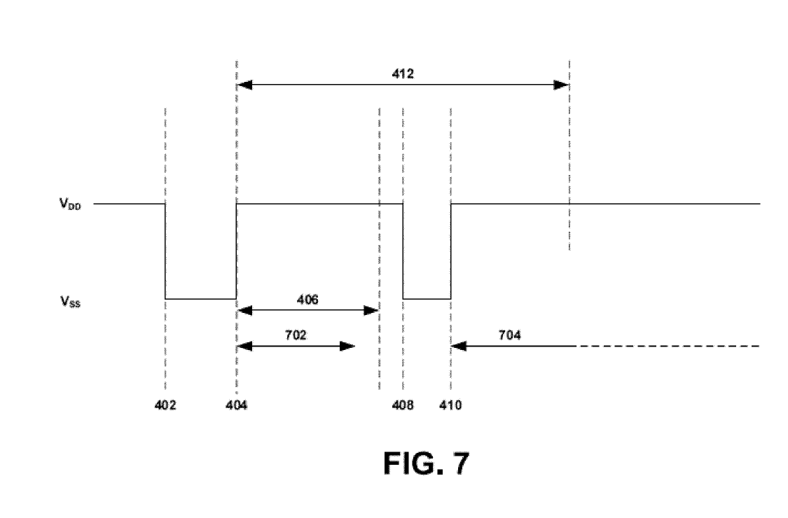

A variety of tests can be performed in this configuration. Referring to FIG. 7, due to the double button press safeguards against accidental dosing, there are several opportunities to apply switch integrity testing. After a button release at time 404, switch 302 is ignored until predetermined minimum time interval 406 has elapsed, during this period the integrity of switch 302 and its interfaces can be tested. As long as the test takes less than the minimum time interval 406, a short test (e.g. a fast analog test or a digital test) can be performed. In some embodiments, a fast analog test is performed. Depicted in FIG. 7 is time span 702 which is the time a short test can be performed. After the second button release at time 410, another test (e.g. a digital or a fast or slow analog test) can take place during the delivery of the therapeutic agent, because during this period of time any signal by switch 302 can be ignored. The second test is depicted in FIG. 7 during time span 704. Again, although V_{DD} and V_{SS} are used for illustrative purposes in FIG. 7, any logical high (V_{H}) can be used instead of V_{DD} and any logical low (V_{L}) can be used instead of V_{SS}. In some embodiments V_{H}<V_{DD} or V_{L}>V_{SS}. In some embodiments V_{H}<V_{DD} and V_{L}>V_{SS}.

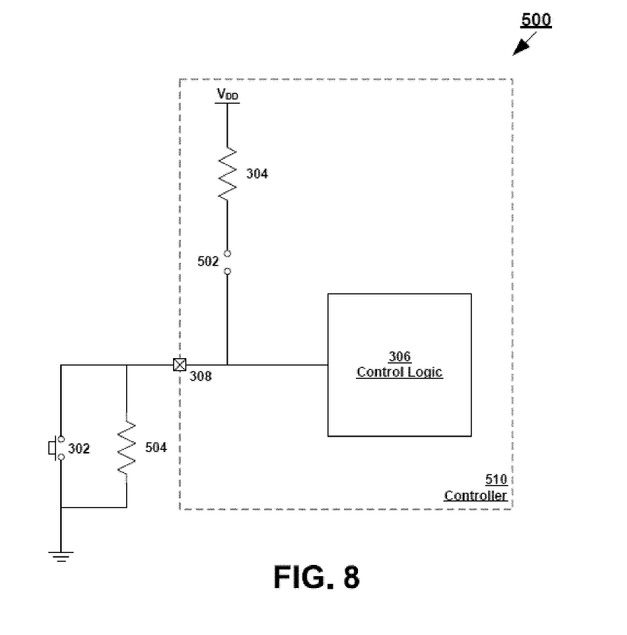

FIG. 8 shows an equivalent circuit configuration of therapeutic agent delivery device 500 during a short interval switch grounding integrity test. During the short interval switch test, switch integrity test output 506 is forced from a high supply voltage state to a low supply voltage state, depicted in FIG. 8 as grounding resistor 504. Additionally switch 502 is opened during the test. During the test resistor 504 acts as a pull down resistor causing the voltage at switch input 308 to drop from V_{DD} to V_{SS}. The rate at which the voltage falls is based on the resistance-capacitance (“RC”) time constant. The resistance in the circuit is furnished by resistor 504 and the capacitance is the capacitance inherent in switch input 308 and circuitry. For example, if controller 510 is implemented in an ASIC mounted to a printed circuit board (PCB), metal traces in the PCB, interface pins, balls or lands in the ASIC package can be major sources of capacitance. Due to experimentation, a nominal capacitance of controller 510 can be determined. Any deviation in the observed decay rate of the voltage seen at switch input 308 can result from resistor 504 being bad, contamination, shorts, open circuits (“opens”), missing or bad PCB traces, or a bad ASIC interface. For example, electrostatic discharge (ESD) during manufacturing, packaging, storage or use could damage the ASIC interface. Again, although V_{DD} and ground are used for illustrative purposes in FIG. 8, any logical high (V_{H}) can be used instead of V_{DD} and any logical low (V_{L}) can be used instead of ground. In some embodiments V_{H}<V_{DD} or V_{L}>ground. In some embodiments V_{H}<V_{DD} and V_{L}>ground.

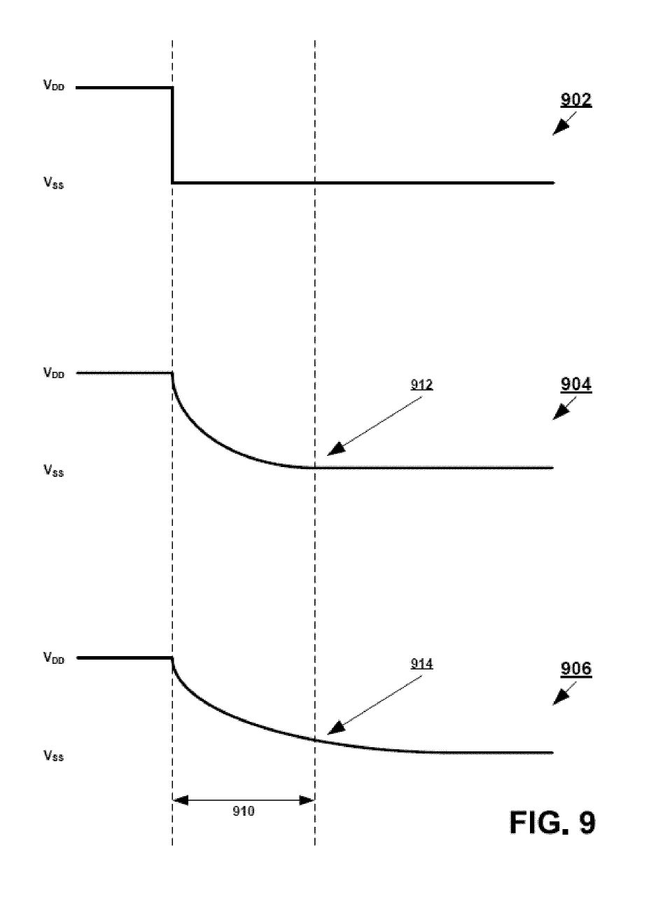

FIG. 9 shows signaling during the short interval switch grounding integrity test. Signal trace 902 is the signal from integrity switch test output 506 which initially begins at V_{DD} and drops abruptly to V_{SS}. Signal trace 904 is the signal observed at switch input 308 for a “good” therapeutic agent delivery device. After predetermined time interval 910 has elapsed after the drop in the voltage of integrity switch test output 506, the signal has decayed to a known value as indicated by arrow 912. However, if the after predetermined time interval 910, the signal as shown by signal trace 906 observed at switch input 308 does not decay as rapidly as expected, to the known value as indicated by arrow 914, there may be excess capacitance or resistance in the test circuit which could indicate the existence of a fault or a precursor of a fault as described above. Again, although V_{DD} and V_{SS} are used for illustrative purposes in FIG. 9, any logical high (V_{H}) can be used instead of V_{DD} and any logical low (V_{L}) can be used instead of V_{SS}. In some embodiments V_{H}<V_{DD} or V_{L}>V_{SS}. In some embodiments V_{H}<V_{DD} and V_{L}>V_{SS}.

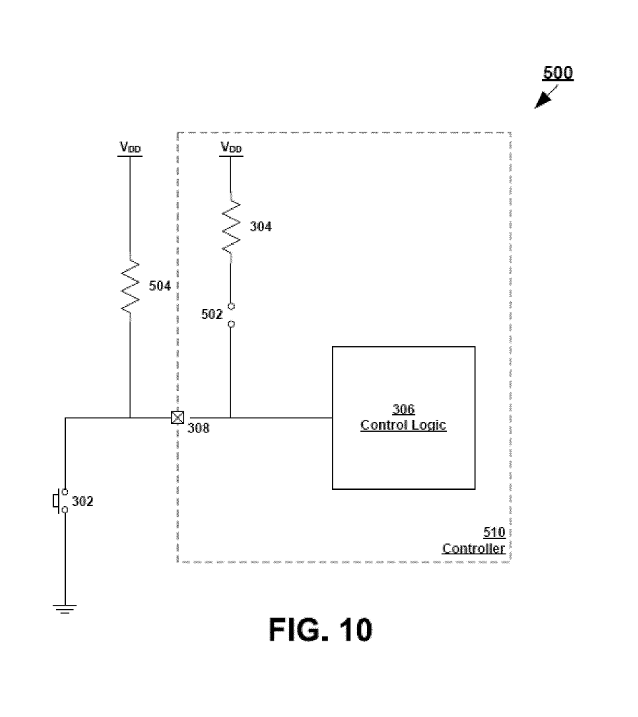

FIG. 10 shows an equivalent circuit configuration of therapeutic agent delivery device 500 during a short interval power switch integrity test. During the short interval switch test, switch integrity test output 506 is forced from a low supply voltage state to a high supply voltage state, depicted in FIG. 10. Once again switch 502 is opened during the test. During the test, resistor 504 acts as a pull up resistor causing the voltage at switch input 308 to rise from V_{SS} to V_{DD}. The rate at which the voltage rises is based on the RC time constant, similar to that described above for the short interval switch grounding integrity test. Once again, the causes of deviation from the nominal RC time constant described above can result from resistor 504 being bad, contamination, shorts, opens, missing or bad PCB traces, or a bad ASIC interface. Again, although V_{DD} and ground are used for illustrative purposes in FIG. 10, any logical high (V_{H}) can be used instead of V_{DD} and any logical low (V_{L}) can be used instead of ground. In some embodiments V_{H}<V_{DD} or V_{L}>ground. In some embodiments V_{H}<V_{DD} and V_{L}>ground.

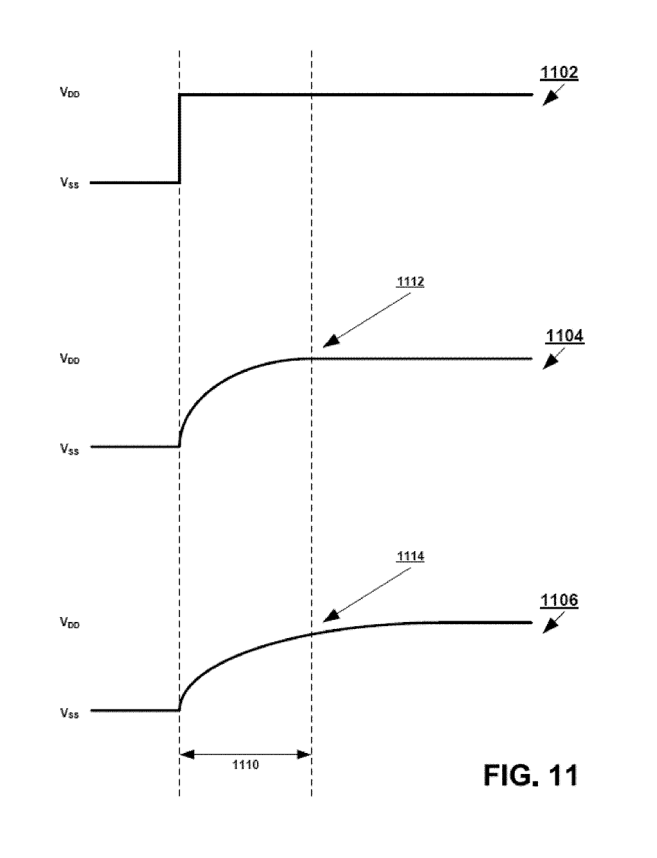

FIG. 11 shows signaling during the short interval power switch integrity test. The signal is logically complementary to that depicted in FIG. 9. Signal trace 1102 is the signal from integrity switch test output 506 which initially begins at V_{SS} and rises abruptly to V_{DD}. Signal trace 1104 is the signal observed at switch input 308 for a “good” therapeutic agent delivery device. After predetermined time interval 1110 has elapsed after the drop in the voltage of integrity switch test output 506, the signal has risen to a known value as indicated by arrow 1112. However, if the after predetermined time interval 910, the signal as shown by signal trace 906 observed at switch input 308 does not rise as rapidly as expected, to the known value as indicated by arrow 1114, there may be excess capacitance or resistance in the test circuit which could indicate the existence of a fault or a precursor of a fault as described above. It is noted that where testing is conducted after a second button push, e.g. as in some embodiments employing digital testing, there need not be any timing element; and in some such embodiments there is no timing element. Again, although V_{DD} and V_{SS} are used for illustrative purposes in FIG. 11, any logical high (V_{H}) can be used instead of V_{DD} and any logical low (V_{L}) can be used instead of V_{SS}. In some embodiments V_{H}<V_{DD} or V_{L}>V_{SS}. In some embodiments V_{H}<V_{DD} and V_{L}>V_{SS}.

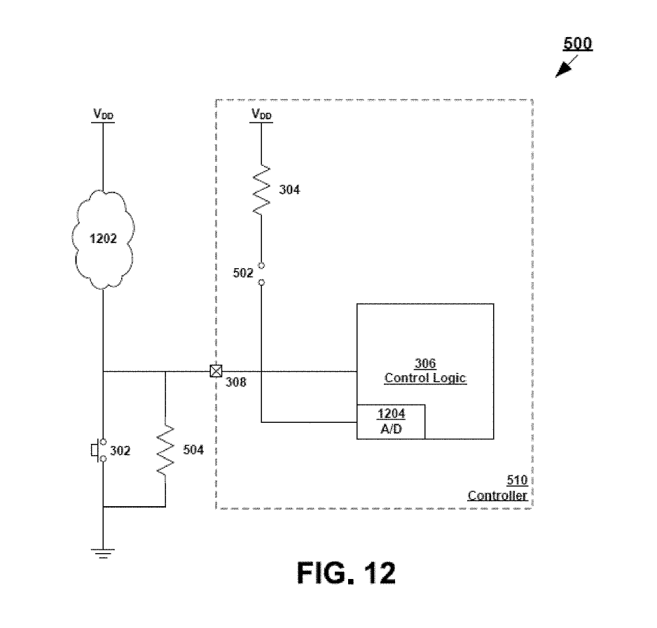

FIG. 12 shows an equivalent circuit configuration of therapeutic agent delivery device 500 during an analog switch grounding integrity test. The equivalent circuit configuration shown in FIG. 12 is essentially the same configuration as that depicted in FIG. 8. Additionally control logic 306 further comprises a means for measuring the voltage at switch input 308. In the depicted embodiment, the means for measuring voltage is analog to digital converter (“ADC”) 1204, however other methods for measuring voltage can be implemented, such as the use of a set of comparator circuits in place of the ADC to measure the voltage level of the analog signal compared to a comparator threshold. As in FIG. 8, switch integrity test output 506 is forced down to a low supply voltage state, so resistor 504 acts as a pull down resistor. If contamination or corrosion (shown as 1202) exists between switch 302, switch input 308 or connecting wirings and a high power supply source such as a power line metal trace, the contamination or corrosion may act as a resistor pulling up against resistor 504 resulting in a voltage divider. The result is that resistor 504 would not be able to completely pull down the voltage at switch input 308 down to V_{SS}. If the voltage that switch input 308 fails to settle at V_{SS}, then contamination, corrosion or other corruption of the apparatus is causing a short between the switch 302 and/or switch input 308 and a high power supply source. Again, although V_{DD} and V_{SS} are used for illustrative purposes in FIG. 12, any logical high (V_{H}) can be used instead of V_{DD} and any logical low (V_{L}) can be used instead of V_{SS}. In some embodiments V_{H}<V_{DD} or V_{L}>V_{SS}. In some embodiments V_{H}<V_{DD} and V_{L}>V_{SS}.

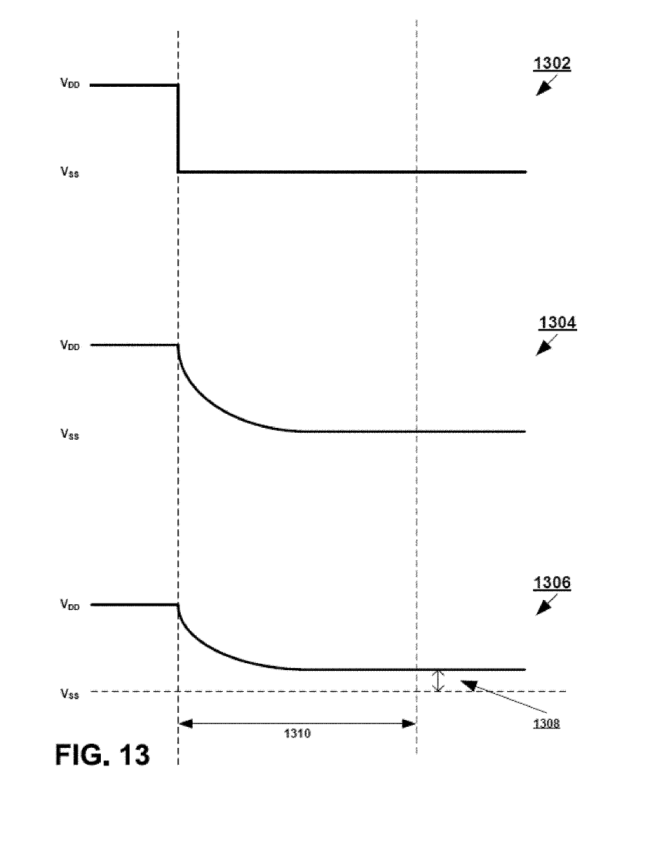

FIG. 13 shows signaling during the long interval analog switch grounding integrity test. (Although reference is made to a long interval analog grounding integrity test, the test may be made short interval by adjusting the number of data points collected.) Signal trace 1302 is the signal from integrity switch test output 506 which initially begins at V_{DD} and drops abruptly to V_{SS}. Signal trace 1304 is the signal observed at switch input 308 for a “good” therapeutic agent delivery device. After predetermined time interval 1310 has elapsed after the drop in the voltage of integrity switch test output 506, the signal has decayed to its final value. Predetermined interval 1310 differs from predetermined interval 910 shown in FIG. 9. Because the objective of the short interval test is to measure the rate of decay, predetermined interval 910 should be short enough so that any change in the RC time constant would be observed. In contrast, predetermined interval 1310 should be long enough so that the signal observed at switch input 308 should have decayed to a steady state voltage regardless of the RC time constant (or at least within a reasonable range of RC time constants). Signal trace 1306 is the signal observed at switch input 308 for a therapeutic delivery agent when corruption or some other source causes a short between a high power supply and switch 302 and/or switch input 308. The discrepancy between the steady state voltage and V_{SS} is indicated by arrow 1308. Again, although V_{DD} and V_{SS} are used for illustrative purposes in FIG. 13, any logical high (V_{H}) can be used instead of V_{DD} and any logical low (V_{L}) can be used instead of V_{SS}. In some embodiments V_{H}<V_{DD} or V_{L}>V_{SS}. In some embodiments V_{H}<V_{DD} and V_{L}>V_{SS}

Operationally, after predetermined time interval 1310, control logic 306 measures the voltage at switch input 308. If the steady state voltage exceeds a given threshold, a fault can be indicated by controller 510. Additionally or alternatively, if the steady state voltage exceeds a second threshold a precursor to a fault can be indicated and appropriate action can be taken by controller 510.

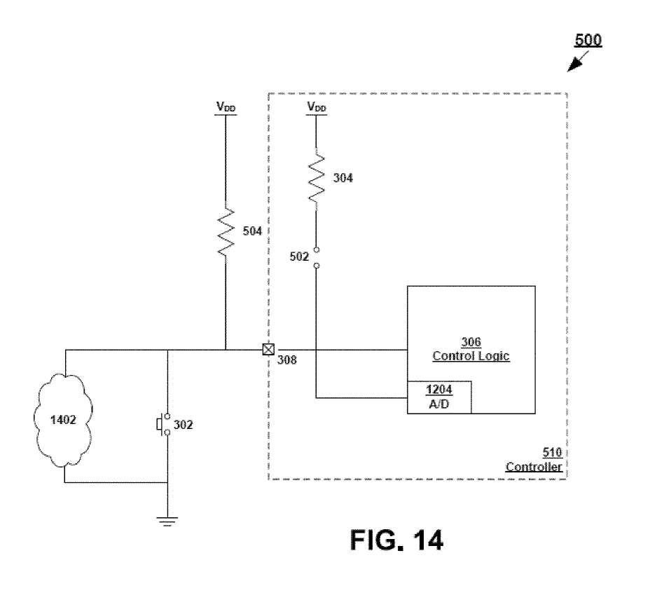

FIG. 14 shows an equivalent circuit configuration of therapeutic agent delivery device 500 during a long interval analog power switch integrity test. The equivalent circuit configuration shown in FIG. 14 is essentially the same configuration as that depicted in FIG. 10. Once again control logic 306 further comprises a means for measuring the voltage at switch input 308. As in FIG. 10, switch integrity test output 506 is forced up to a high supply voltage state, so resistor 504 acts as a pull up resistor. If contamination or corrosion (shown as 1402) exists between switch 302, switch input 308 or connecting wirings and a low power supply source such as a ground trace, or if contamination or corrosion intrudes between the two poles on switch 302 causing switch 302 to short, the contamination or corrosion may act as a resistor pulling down against resistor 504 resulting in a voltage divider. The result is that resistor 504 would not be able to completely pull up the voltage at switch input 308 up to V_{DD}. If the voltage that switch input 308 fails to settle at V_{DD}, then contamination, corrosion or other corruption of the apparatus is causing a short to a low power supply source. Again, although V_{DD} and V_{SS} are used for illustrative purposes in FIG. 14, any logical high (V_{H}) can be used instead of V_{DD} and any logical low (V_{L}) can be used instead of V_{SS}. In some embodiments V_{H}<V_{DD} or V_{L}>V_{SS}. In some embodiments V_{H}<V_{DD} and V_{L}>V_{SS}.

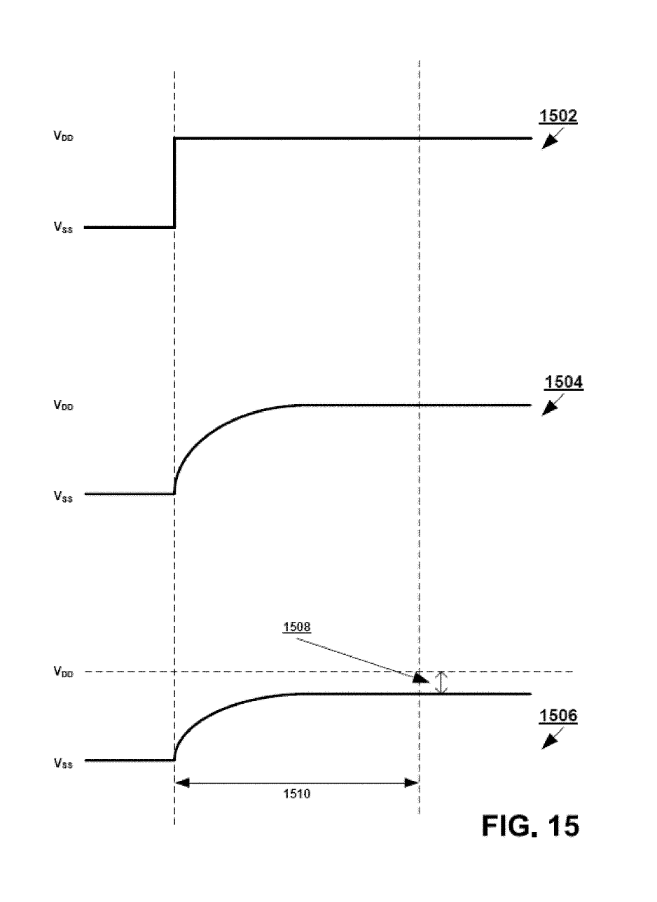

FIG. 15 shows signaling during the long interval analog power switch integrity test. Signal trace 1502 is the signal from integrity switch test output 506 which initially begins at V_{SS} and rises abruptly to V_{DD}. Signal trace 1504 is the signal observed at switch input 308 for a “good” therapeutic agent delivery device. After predetermined time interval 1510 has elapsed after the rise in the voltage of integrity switch test output 506, the signal has risen to its final value. Once again, predetermined interval 1510 differs from predetermined interval 1110 shown in FIG. 11, for reasons similar to the difference between predetermined interval 1310 and predetermined interval 910. Signal trace 1506 is the signal observed at switch input 308 for a therapeutic delivery agent when corruption or some other source causes a short between a low power supply and switch 302 and/or switch input 308. The discrepancy between the steady state voltage and V_{DD} is indicated by arrow 1508. Again, although V_{DD} and V_{SS} are used for illustrative purposes in FIG. 15, any logical high (V_{H}) can be used instead of V_{DD} and any logical low (V_{L}) can be used instead of V_{SS}. In some embodiments V_{H}<V_{DD} or V_{L}>V_{SS}. In some embodiments V_{H}<V_{DD} and V_{L}>V_{SS}.

Operationally, after predetermined time interval 1510, control logic 306 measures the voltage at switch input 308. If the voltage differential between the steady state voltage and V_{DD} exceeds a given threshold, a fault can be indicated by controller 510. Additionally or alternatively, if the voltage differential exceeds a second threshold a precursor to a fault can be indicated and appropriate action can be taken by controller 510.

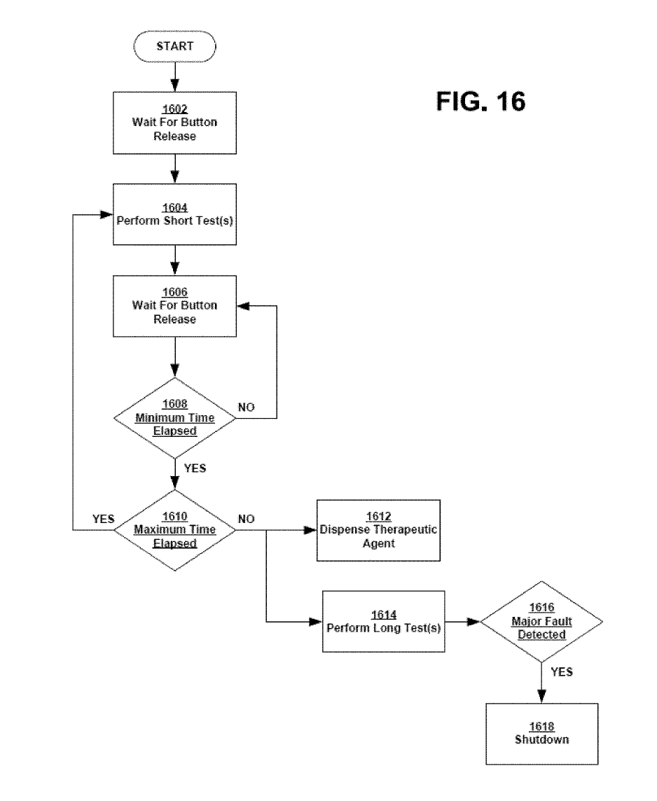

FIG. 16 shows a flow chart of the dosing operation of an embodiment of a therapeutic agent delivery device with switch integrity testing. At step 1602, the device waits for a button release. This corresponds to waiting for event 404 in FIG. 7. At step 1604 after the button has been released one or more short switch integrity tests can be performed such as those described above in FIGS. 8-11. At step 1606, the device waits for a second button release. After the button has been released, at step 1608, a determination is made as to whether the second button press has occurred within the predetermined minimum time interval. If it has not, the last button release is ignored and the device returns to step 1606 where it waits for another button release. If it has, a determination is made as to whether the maximum time interval since the first button release has elapsed. If it has, the second button release is treated as the first hence the device returns to step 1604. If the maximum time has not elapsed, at step 1612, delivery of the therapeutic agent begins. (Although not specifically depicted in FIG. 16, it is to be understood that one or more switch integrity checks may be performed between step 1610 and step 1612, such as a digital switch integrity check or a fast analog integrity check.) Concurrently with delivery of therapeutic agent, the device can perform one or more optional long switch integrity tests at step 1614. Concurrently, a determination is made at step 1616 as to whether a fault with sufficient severity to warrant the shutdown of the device has occurred. If so the device shuts down at step 1618.

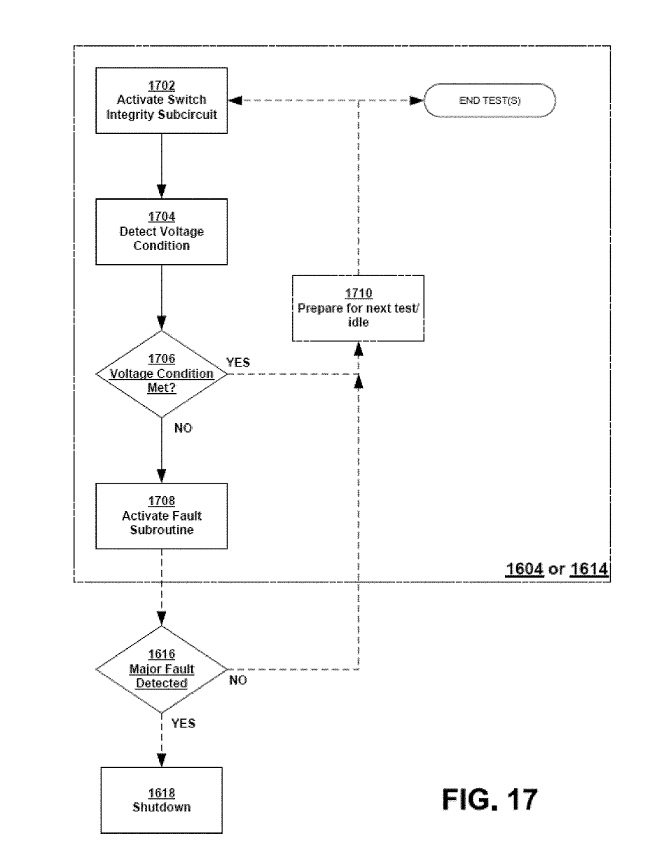

FIG. 17 shows exemplary embodiment of a switch integrity testing process. The flowchart shown is representative of typical switch integrity processes which be used in steps 1604 and/or step 1614. At step 1702, device 500 activates its switch integrity subcircuit. In the examples given above, this can include opening switch 502, setting the switch integrity test output to a predetermined voltage such as V_{DD} or V_{SS} and/or optionally powering on or activating ADC 1204 such as in the configurations shown in FIGS. 12 and 14. In some embodiments, the ADC circuitry could be powered off when not testing to save power. At step 1704, one or more predetermined voltage conditions are tested for. Examples of these conditions are described above in FIGS. 8-15. For example, in the short tests described in FIGS. 8-11, after a predetermined time interval has elapsed after the switch integrity test output is set to the predetermined voltage, the voltage at switch input 308 is measured. If the measured voltage has risen or decayed to the expected voltage, a voltage condition is deemed to be detected. In another example, in the long tests described in FIGS. 12-16, after a predetermined time interval has elapsed after the switch integrity test output is set to the predetermined voltage, the voltage at switch input 308 is measured. If a discrepancy exists between the predetermined voltage and the measured voltage then a voltage condition is deemed to be detected.

At step 1706 a determination is made as to whether a predetermined voltage condition was detected, if so at step 1708 a fault subroutine is activated. More specifically, each predetermined voltage condition is associated with a fault or a precursor to a fault. The fault subroutine can take one or more courses of action depending on the severity of the fault or precursor to a fault. For example, the patient or care provider can be alerted by activating a user alert feature. As previously discussed, the user alert feature can include a variety of means to alert a user that operation of the system is considered compromised. In some embodiments, the device is configured to detect precursors to faults, so the device may activate the user alert even before a fault has been detected that would cause an effect that would be experienced by the patient. The user alert may be an indicator light, such as a colored light emitting diode (LED), an audible tone (such as a repeating “beep”), a readable display (such as a liquid crystal display (LCD)), other user observable indicator, communications to an external monitoring device, (e.g., a wireless transmission to a central console) or combinations of two or more thereof.

In another example, the faults and precursors to faults can be logged in memory. In some such embodiments, the controller detects a certain type of fault, assigns it a fault code, and records the fault code in memory for retrieval at a later time. For instance, the controller may detect and record one of the following conditions: a low voltage at a point and under conditions where a high voltage would be expected for a normally operating circuit; a voltage at a point and under conditions that is higher or lower than the voltage that would be expected for a normally operating circuit; a voltage rise time that is longer or shorter than would be expected for a normally operating circuit; a voltage or current fall time that is longer or shorter than would be expected for a normally operating circuit; or combinations of two or more thereof. The logs can be retrieved in several ways, for example it may be retrieved by a removable memory medium such as flash memory, viewed by a care provider by one or more visual messages on a display device, or transmitted to an external monitoring device.

In another example, when the faults have sufficient severity pose a risk to a patient, the device can be deactivated such as by irreversibly decoupling the voltage supply from the drug delivery circuit, shorting a power cell to ground, fusing a fusible link in the circuit, by means of software logic, etc., as described herein.

In another example, the fault subroutine can perform a combination of the actions described. For example, initially, precursors to faults are logged, but as the severity of the potential faults increases, a user alert is issued. Finally, when potential faults become actual faults and the severity is sufficiently high, the device shuts down at step 1618.

If no voltage condition is found at step 1706 or after the voltage condition is processed at step 1708, optionally the switch integrity process can proceed to step 1710 where either the device prepares for the next test or prepares to end the final test. In the former case, the device may set the switch integrity test output to another voltage. For example, in preparation for one of the grounding tests described above in FIGS. 8-9, 12-13, the switch integrity test output could be set to V_{DD} so that when the grounding tests begins in step 1702 the switch integrity test output can be driven down to V_{SS} to initiate the test. However, this can be minimized by proper selection of tests. For example, if the power tests and the ground tests are alternated, there is no need to set the switch integrity test output to another voltage as each tests leaves the switch integrity test output in the appropriate voltage to initiate the other test. In the latter case at step 1710, the device can deactivate the switch integrity subcircuit, for example the switch integrity test output can be set to its non-test default state which can be either the high supply voltage or the low supply voltage. Alternatively, the switch integrity test output could be left floating. Additionally switch 502 is closed so that resistor 304 can resume its pull up function.

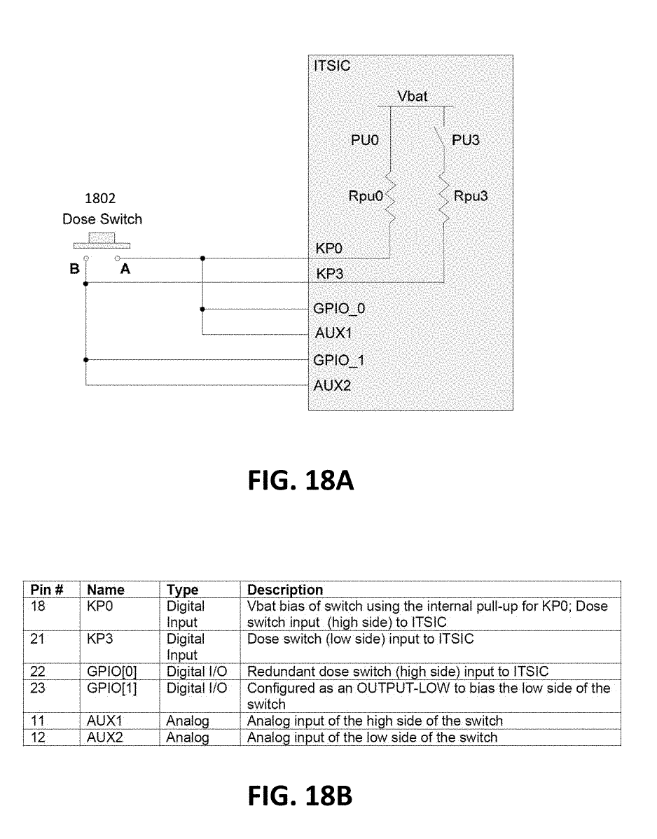

As described above, any of the apparatuses and methods described herein may be configured to perform both analog and digital switch validation of the dose switch. FIG. 18A illustrates one example of a circuit description for a drug delivery device that performs both analog and digital switch validation.

For example, a normally-open switch (e.g., a momentary-contact push-button switch) (SW1) is located in the circuit. In FIG. 18A, the SW1 switch is located on the IT101 circuit board, and is referred to as the dose switch. Each side of the switch is directly connected to three separate lines on the circuit (IC), which contains the control logic. The Aux1, KP0 and GPIO0 lines are on one side of the dose switch and Aux2, KP3, and GPIO2 are on the other side of the dose switch. These connections allow the controller (e.g., “ITSIC”) to confirm that the dose switch is operating properly. Any appropriate dose switch may be used. For example, the dose switch may be a mechanical switch configured as a button having a round metal snap dome, with a characteristically short contact bounce. No electrical de-bouncing is required for such an example, although switches with electrical de-bouncing could be used. FIGS. 18A and 18B show the dose switch connection design and descriptions of nodes.

For example, in FIG. 18A, the high side of the switch (“A”) includes nodes for the first power input line (KP0), the first digital test input line (GPIO_0), the first analog test input line (AUX1). The low side of the switch (“B”) includes nodes for the second power input line (KP3), the second digital test input line (GPIO_1), and the second analog test input line (AUX2). The battery (Vbat) is also shown connected to the KP0 and KP3 lines, including pull-up resistors (Rpu0 and Rpu3). The analog and digital test input lines all connect to the controller (ITSIC) where they be analyzed to perform the digital validation (using GPIO_0 and GPIO_1) and analog validation (using AUX1 and AUX2). In this example the same controller/processor is used; different processors, including sub-processors, may be used.

Three separate techniques (procedures) may provide redundancy and enable demonstration of the validation method to a high degree of certainty, particularly when all three are employed and integrated as part of the apparatus. Specifically, button sampling, analog switch validation, and digital switch validation may all be included.

Button sampling (including a button sampling procedure) may be used to detect button pressing and release. In particular, button sampling may include the use series of sequential state tests to determine when the button is in a stable configuration (e.g., pressed or released) by comparing sequential samples taken over a short period of time. Rapid changes in the state indicate that the button is not in a stable (“pushed” or “released”) state. For example, to detect transitions of a button input and to filter out noise signals caused by switch bounce or other events, button inputs may be sampled periodically, e.g., every n ms (e.g., where n may be 2 ms, 3 ms, 4 ms, 5 ms, 6 ms, 7 ms, 8 ms, 9 ms, 10 ms, between about 1-20 ms, 1-10 ms, 2-10 ms, etc.). The sampling frequency may provide responsiveness to user inputs. The sampled data (button input sample data) may be buffered into a circular buffer that holds a predetermined number of samples (e.g., 4 samples, 5 samples, 6 samples, 7 samples, 8 samples, 9 samples, 10 samples, 11 samples, 12 samples, 13 samples, etc.). The most recent samples (e.g., the four most recent samples) may be used by a button sampling test (which may be implemented in hardware, software, firmware, or some combination thereof) to determine the state of the button. The state of the button is determined (e.g., as open or closed) when all of the most recent samples (e.g., all four samples) are the same state. This distinguishes a stable button state from a mechanical switch bounce or electrical noise. If the buffer contains a mix of low and high sample values, the signal may be determined to be a result of switch bounce or electrical noise and the apparatus may ignore the signal.

Press and release transitions may be detected, and upon each transition, the state of the buttons may be sampled (e.g., at a rate of approximately 50 ms). For example, a release transition may be confirmed by detection of four depressed states flowed by four released states, and a press transition may be confirmed by the opposite sequence. If the button is sampled every 8 ms and 4 samples are examined within the rolling window, the result is approximately 65 ms of sampling time to identify a valid button state transition.

Using two separate switch validation techniques/pathways (e.g., analog and digital switch validation) may provide redundancy and enable demonstration of the validation to a high degree of certainty in a way that is surprisingly better than a single validation technique/path. The analog switch validation test and the digital switch validation test are both performed, or may both be performed; in some variations both tests are performed only when one of the test is performed first and passes (e.g., is true). For example, the analog switch validation may be performed only if the digital switch validation is true, or vice/versa.

The controller, which may include firmware, hardware and/or software, typically controls and monitors the dose switch circuit using both digital and analog signals. An analog portion of the dose switch circuit may be used to monitor analog voltages on both sides of the dose switch (e.g., the high, “A”, and low, “B”, sides). A digital portion of the dose switch circuit may be used for switch bias control and digital monitoring on both sides of the switch. In the example shown in FIG. 18A, software may configure the keypad input pull-up KP0 and GPIO[1] to establish a Vbat bias across the switch 1802, as shown. KP3 and GPIO[0] may be used to monitor the digital state of the switch.

An analog switch validation test may measure the voltage levels on both the high and low sides of the dose button switch in order to detect potential problems that could lead to erroneous switch readings. Under normal conditions with the switch open, voltage on the high side of the switch will be slightly less than battery voltage, after accounting for the small voltage drop caused by the electronic components connected to the switch circuit. Under normal conditions, the voltage on the low side of the switch will be very close to ground. Some conditions, such as contamination or corrosion, can cause the high-side voltage to drop, or the low-side voltage to rise. If the high-side voltage falls to less than a predetermined high-side threshold, such as some predetermined high-side fraction of the battery voltage (e.g., 0.8×battery voltage), or the low-side voltage rises to greater than some predetermined low-side threshold, such as a predetermined low-side fraction of the battery voltage (e.g., 0.2×battery voltage), then the switch input may fall in a range of indeterminate digital logic level with respect to the digital switch input. A switch voltage in this range could result in erroneous switch readings, which could manifest as false button transitions that were not initiated by the user, and therefore improper dosage. An analog switch validation test may therefore detect a condition before the switch voltage levels reach the point where erroneous readings could occur.

The analog switch validation test may be run when the switch is in its normally-open condition, so that the high- and low-side voltages can both be measured. Any change in the switch state while the test is running could cause the test to falsely fail due to measurement of the high-side voltage while the switch is closed. Since a user may press or release the button at any time, the apparatus may be configured to run the test in such a way to avoid interference with normal operation, e.g., allowing a button push, or more likely a pair of button pushes, at any time without interfering with the analog and/or digital switch validation. The apparatus and methods described herein may take advantage of the fact that there are mechanical and human limits on the minimum time between button presses, and thus the point where the switch state is known to be open with the greatest certainty is immediately following a detected release of the button. Thus the analog and/or digital switch validation may be performed following one or more button pushing events, or more likely button release events.

For example, an analog switch validation test may be performed immediately following the second button release of a double-press that meets the criteria for a dose initiation sequence. An analog switch validation may use an analog-to-digital converter (ADC), e.g., part of the controller/processor (e.g., ITSIC), to make sequential measurements of the high-side voltage and the low-side voltage. For example, an ADC may be configured to sample for 6.25 ms for each measurement. If the voltage on the high side of the switch is less than or equal to the high side predetermined threshold (e.g., 0.8×battery voltage), or if the voltage on the low side is greater than or equal to the low side predetermined threshold (e.g., 0.2×battery voltage), the test fails. The switch high and low limits may be calculated and stored each time the battery voltage is measured for a battery voltage test.

A digital switch validation test is generally also performed by the apparatus and methods describe herein. A digital switch validation test may be similar in purpose to the analog switch validation test, but is generally simpler, faster, and coarser in its measurements. The test may use secondary digital inputs (e.g., GPIO[0] and GPIO[1] in FIGS. 18A and 18B), connected to each side of the dose switch 1802, to confirm the digital logic levels while the switch is open (e.g., button not depressed). These “secondary” digital inputs (e.g., first and second digital test input lines) may be of the same type as the primary digital inputs, and the corresponding values of these digital inputs are expected to match. For example, the first (high side) digital input test line should have the same logical value as the first input line connected to the battery and the second (low side) digital input test line should have the same logical value as the second input line.

The digital switch validation test may be run either before, during or after an analog switch validation test. The performance of the analog switch validation test may depend on a successful digital switch validation test, or vice versa. For example, an analog switch validation test may be performed after a successful digital switch validation test following the second button release of a double-press that meets the criteria for a dose initiation sequence. For example, if the secondary digital input on the high side of the switch is low, or if the secondary digital input on the low side of the switch is high, the digital switch validation test fails, and the system may initiate a failure mode (e.g., a digital switch validation failure mode); if the secondary digital input on the high side of the switch is high, and if the secondary digital input on the low side of the switch is low, the digital switch validation test passes, and the system may then perform an analog switch validation, as described above. If the analog switch validation test fails, then the system may also initiate a failure mode (e.g., an analog switch validation failure mode). The failure mode may include locking the device (to prevent further activations), shutting the device down, restarting the device, issuing an alert/warning (e.g., buzzer, alarm, etc.), disconnecting the battery from the circuit, or some combination of these. For example, if the analog switch validation test fails, the apparatus may enter into an end of life mode.

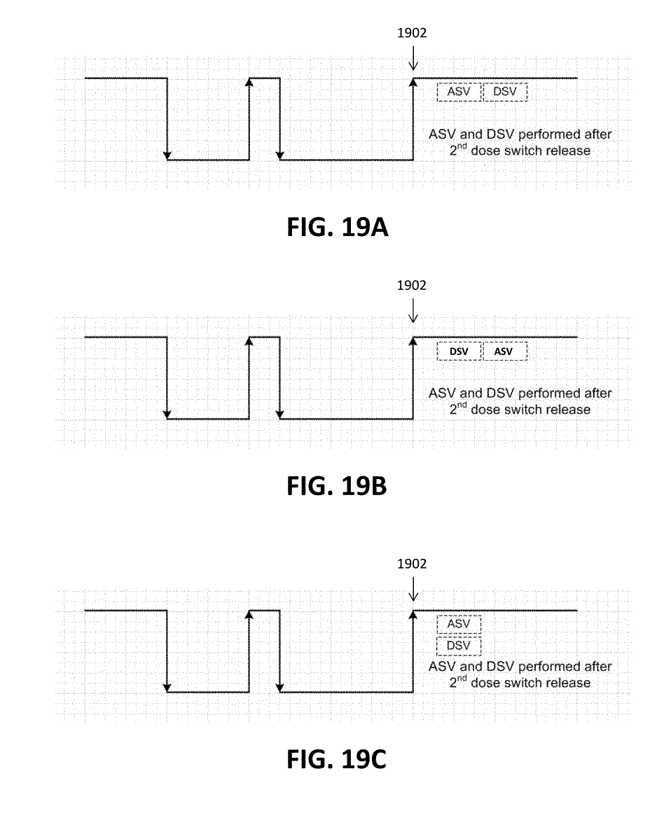

FIGS. 19A-19C illustrate variations on the timing of a dose switch activation sequence for an apparatus or method that is configured to perform both analog and digital switch validation tests. In FIGS. 19A-19C, following a second activation of a dose switch within a predetermined time period 1902, both the switch validation tests are performed. In FIG. 19A, the analog switch validation (ASV) test is performed first, followed by the digital switch validation (DSV) test. The digital switch validation test may be performed if the analog switch validation test is good (e.g., if the high and low sides of the switch are within the acceptable voltage ranges set by the predetermined thresholds (e.g., >0.8×Vbat on the high side and <0.2 Vbat on the low side). Both the analog and the digital switch validation tests may be performed within a window of time following release of the switch (e.g., following the second release within a switching time period). The window of time may begin immediately or shortly after detecting the release of the switch and extend for a period of time during which it is impossible or highly unlikely that a subject could push the button again. For example the switch validation tests may be performed before the test period (test window) has ended (e.g., 500 ms, 400 ms, 300 ms, 200 ms, 150 ms, 100 ms, 50 ms, etc.).

In FIG. 19B, the digital switch validation (DSV) test is performed first, followed by the analog switch validation (ASV) test. For example, the analog switch validation may be performed only if the digital switch validation passes (e.g., the high side is a logical 1 and/or matches the high-side voltage input from the first input line connected to the battery, and the low side is a logical 0 and/or matches the low-side voltage input from the opposite input line). If the digital switch validation does not pass, the device may enter a first failure mode (e.g., restarting, and/or incrementing a counter or flag indicating failure of the digital switch validation, shutting down, etc.). If the digital switch validation passes, and the subsequent analog switch validation passes, then the dose may be delivered; however, if the digital switch validation passes but the analog switch validation does not pass, then the device may enter into a second failure mode (e.g., shutting the device down, restarting the device, issuing an alert/warning, disconnecting the battery from the circuit, or some combination of these). The first and second failure modes may be the same. In some variations, the first and second failure modes are different. For example, if the digital switch validation test fails, the software may ignore that dose request and remains in Ready mode (first failure mode), and if the analog switch validation test fails, the apparatus may enter into an end of life failure mode (EOL mode). In some variations, the analog switch validation test is more sensitive (e.g., uses more sensitive circuitry) than the digital switch validation test. Passing the analog switch validation test may indicate that the circuitry is intact; failure of the analog switch validation test may indicate a failure of the circuitry. In such instances, failure of the analog switch validation test may therefore cause the apparatus to enter into EOL (end of life) mode. Passing the digital switch validation test may also (redundantly) indicate that the circuitry is intact, but failure of the digital switch validation test may not necessarily indicate failure of the circuitry. Failure of the digital switch validation test may also be a result of temporary electrical noise signals. Performing the analog switch validation test before the digital switch validation test may therefore prevent false positive failures of the digital switch validation test from disabling the system by entry to EOL mode.

FIG. 19C illustrates another variation in which the analog and digital switch validation modes are performed at the same time, or approximately the same time, following the second release of the does switch detected during the allowable time period (e.g., the time period when to activations of the does switch indicate a dose is requested).

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The present disclosure describes a two-part electrotransport therapeutic agent delivery device, such as an iontophoresis device, in which the two parts of the device are provided separately and assembled to form a unitary, powered-on device at the point of use—that is to say just prior to use. One part of the device, which may be referred to herein as the electrical module, holds essentially all of the circuitry, as well as the power source (e.g. battery), for the device; and the other part, which may be referred to herein as the reservoir module, contains the therapeutic agent to be delivered along with electrodes and hydrogels necessary to deliver the therapeutic agent to a patient. The device is configured such that the power source is kept electrically isolated from the rest of the circuitry in the electrical module until the electrical module is combined with the reservoir module. Thus, embodiments provided herein permit the combination of the electrical module and the reservoir module, whereby in a single action the two modules form a single unit and the battery is introduced into the circuitry, thereby powering on the device, in a single action by the user.

Unless otherwise indicated, singular forms “a”, “an” and “the” are intended to include plural referents. Thus, for example, reference to “a polymer” includes a single polymer as well as a mixture of two or more different polymers, “a contact” may refer to plural contacts, “a post” may indicate plural posts, etc.

As used herein, the term “user” indicates anyone who uses the device, whether a healthcare professional, a patient, or other individual, with the aim of delivering a therapeutic agent to a patient.

As used herein, the term simultaneous, and grammatical variants thereof, indicates that two or more events occur at about the same time and/or that they occur without any intervening step. For example, when connection of the modules occurs simultaneously with connection of the battery into the circuit, the term “simultaneously” indicates that when the modules are connected, the battery is connected into the circuit at about the same time, in a single action by the user, and that there is no additional step necessary on the part of the user to connect the battery to the circuit. The term “substantially simultaneous” and grammatical variants indicates that two events occur at about the same time and no significant action is required by the user between the two events. For the sake of illustration only, such a significant action could be the activation of a separate switch (other than the herein-described power-on switches), removal of a tab, or other action to connect the battery in the electrical module to the circuitry therein upon connection of the two modules to one another.

Unless otherwise modified herein, the term “to break” and grammatical variants thereof refers to destroying or deforming something to the point that it is no longer operable for its intended purpose.

The present disclosure provides an electrotransport device that is assembled before use for electrotransport delivery of ionic compounds (e.g., ionic drugs such as fentanyl and analogs, polypeptides, and the like) through a surface, such as skin. The electrotransport device comprises a top or upper portion, herein referred to as an electrical module, and a bottom or lower portion, herein referred to as a reservoir module. The electrical module contains circuitry (e.g. a printed circuit board), a power source (e.g. a battery), one or more power-on switches and such other circuitry as may be deemed desirable for operation of the device (such as an activation switch, a controller, a liquid crystal diode (LCD) display, a connector, a light emitting diode (LED), an audible indicator (e.g. a sound transducer), or combinations thereof), as well as electrical output contacts for electrically connecting the electrical module to a reservoir module. When obtained by the user, the electrical module is separated from the reservoir module. In this state, the battery is maintained outside of the electrical circuit (though within the electrical module), thereby preventing the battery from discharging through the circuit prior to use. Because the battery is electrically isolated from the circuit prior to combining the electrical and reservoir modules, the circuitry has essentially no electrical charge applied to it prior to combination of the two modules, rendering the circuitry far less susceptible to corrosion than if the battery were in the circuit.

The reservoir module contains electrodes and reservoirs for delivery of therapeutic agent to a patient. At least one reservoir contains the therapeutic agent to be delivered. At least one counter reservoir is provided, which generally contains no therapeutic agent, though in some embodiments it is possible for the counter reservoir to contain therapeutic agent. Prior to being connected to the electrical module, the reservoir module is maintained both physically and electrically isolated from the electrical module. For example, one or both of the modules may be sealed in a pouch, such as a plastic or foil pouch, in order to prevent contamination with water, particulates, vapors, etc. As a non-limiting example, both the electrical and the reservoir modules may be sealed in the same pouch. As a further non-limiting example, the reservoir module may be sealed in a pouch and the electrical module left outside the sealed pouch. In other non-limiting examples, the two modules may be sealed in separate pouches.

Prior to use (e.g. just prior to use) the electrical module is combined with the reservoir module to form a single unit, which in a single action, connects the battery into the circuit and powers the device on. The terms “prior to use” and “just prior to use” are described in more detail hereinafter. In general, these terms are intended to indicate that the two parts of the device are combined by a user, and that the device is then used to deliver therapeutic agent to a patient within a predetermined window of time—e.g. from 0 to 8 hrs or from 0 to 72 hours—after the two parts of the device are combined. This predetermined window of time may vary, depending upon the therapeutic agent, the amount of agent to be delivered, requirements of various regulatory agencies, etc. For the sake of clarity, it is to be understood that combination of the electrical and reservoir modules is postponed after manufacture and is carried out at the point of use so that during shipping and storage the power source enclosed within the electrical module is electrically isolated from the circuitry until the two modules are combined by the user.

As stated before, combination of the electrical and reservoir modules connects the battery into the circuit to achieve a powered on state, without any additional action required on the part of the user. For example, there is no need for the user to activate a power switch or remove a tab in order to connect the battery into the circuit. Once the two modules have been properly combined, power is supplied to the circuitry. The circuitry can then operate normally. Normal operation may include various circuitry tests, operation of various indicators (such as the aforementioned LCD, LED and sound transducers), setting of various logic flags, detection of error states and/or logic flags, etc. Normal operation also includes reception of an activation signal, e.g. through an activation button or switch, and providing power to the electrodes through electrical outputs connected to electrical inputs on the reservoir module.

In addition to reducing corrosion and battery discharge prior to use, another advantage of the device is that the electrical outputs from the electrical module and inputs to the reservoir module (i.e. the contacts between the two modules) are electrically and physically separated from the power-on switches that connect the battery into the circuit. This is advantageous, at least because it allows the power-on switches, which connect the battery into the circuit, to be kept entirely internal to the electrical module. This in turn allows the contacts that comprise the power-on switches to be kept contaminant-free, as the electrical module is at least in some embodiments sealed against contaminants, such as water (including water vapor) and/or particulates. As described herein, a power-on switch is closed by an actuator through an elastomeric seal, which permits the battery to be connected into the circuit without the contacts that comprise the switch being exposed to the environment external to the electrical module.

In some embodiments, two or more power-on switches are employed. In some particular embodiments, the power-on switches are physically remote from one another—e.g. on the order of from 0.1 cm to several cm. In some embodiments, the switches are at least 0.5 cm from one another.

As the two modules form a unitary device, they advantageously include one or more mechanical coupler pairs to hold the two modules together. Such coupler pairs can include snap-snap receptacle pairs, which are in some embodiments designed to become inoperative (deform and/or break) if the two modules are forced apart after they are combined. Thus, devices described herein are well-suited for one time use, as they can be adapted to embody mechanical means for ensuring that the device is used only once.

In some embodiments, the device may alternatively, or additionally, employ electrical means for ensuring that the device is used only once. For example, an electrical means may employ a controller in the electrical module which increments a power-on counter when the device is powered on. In such embodiments, before or after the controller increments the counter, it detects the number of counts on the counter, and if it finds that the power-on counts exceed some predetermined value, it executes a routine to power the device off. As a non-limiting example the counter may initially be set to zero upon manufacturing. The device may then be briefly powered on by an external power supply during post-manufacturing testing, which the controller interprets as one power-on event, and thus increments the power-on counter by 1 count. Then when the device is assembled by the user prior to use, the controller interprets the connection of the battery into the circuit as a power-on event, and increments the power-on counter by 1. The controller then detects the count on the counter. If the count is 2 or less, the controller permits the device to operate normally. If however, the count is 3 or more, the controller initiates a power-off sequence.

As a second, non-limiting example, the counter may initially be set to zero upon manufacturing. The device may then be briefly powered on by an external power supply during post-manufacturing testing, which the controller interprets as one power-on event, and thus increments the power-on counter by 1 count. Then when the device is assembled by the user prior to use, the controller detects the count on the counter. If the count is 1 or less, the controller increments the power-on counter and permits the device to operate normally. If however, the count is 2 or more, the controller initiates a power-off sequence.

Although reference is made here to counting power-on sequences, other events may be counted, either in place of power-on events, in addition to power-on events, or as a proxy for power-on events. In particular,

The power off sequence can be a sequence such as described in U.S. Pat. No. 6,216,003 B1, which is incorporated herein in its entirety.

In some embodiments, the device combines both mechanical (e.g. one-way snaps) and electrical (e.g. power-on counter) means to ensure that the device cannot be used more than once.

A single use may include multiple administrations of a therapeutic agent, e.g. within a particular window of time after the device has been powered on. The duration of time during which therapeutic agent may be administered and/or the number of total doses permitted to be administered by the device may be predetermined and programmed into a controller. Means for controlling the number of doses that may be administered and/or the period during which therapeutic may be administered are described e.g. in U.S. Pat. No. 6,216,003 B1, which is incorporated herein in its entirety. For the sake of clarity, the term “single use” is not intended to limit the device to a single administration of drug. Rather, the term “single use” is intended to exclude use of the device on more than one patient or on more than one occasion; it is also intended to exclude the use of an electrical module with more than one reservoir module and/or the reservoir module with more than one electrical module and/or detachment of the reservoir module from the electrical module and reattachment. Thus, single use feature is in some embodiments employed to prevent the patient or another from saving drug and using it at a later time. In some embodiments, such a feature may be employed to prevent abuse of the therapeutic agent.

In at least some embodiments of the device described herein, the device is configured to prevent contamination of the circuitry before and during use in order to reduce the likelihood of device malfunction. For example, the use environment may include emergency room, operative, post-operative or other medical treatment environments, in which potential particulate and liquid are prevalent. Accordingly, at least some embodiments of the device are configured so that one or more seals are formed in order to exclude ambient contaminants from ingress into the working parts of the device, such as in particular the circuitry. In some embodiments, one or more seals are formed around electrical contacts between the electrical outputs on the electrical module and the electrical inputs on the reservoir module.

In some embodiments, the power-on contacts are sealed from ingress of contaminants, such as particulates and fluids. In particular embodiments, the power-on contacts are sealed before the modules are combined, during the act of combination, and after the two modules are combined. In at least some such cases, the power-on contacts may be actuated (switched to a closed position) by an actuator that acts through an interposed elastomer, which maintains an impermeable seal while at the same time being deformed by an actuator (such as a post or other elongate member) to press the power-on contact into a closed position.

Other seals are possible and may be desirable. For example, a seal may be formed between the two parts (modules) when they are combined.

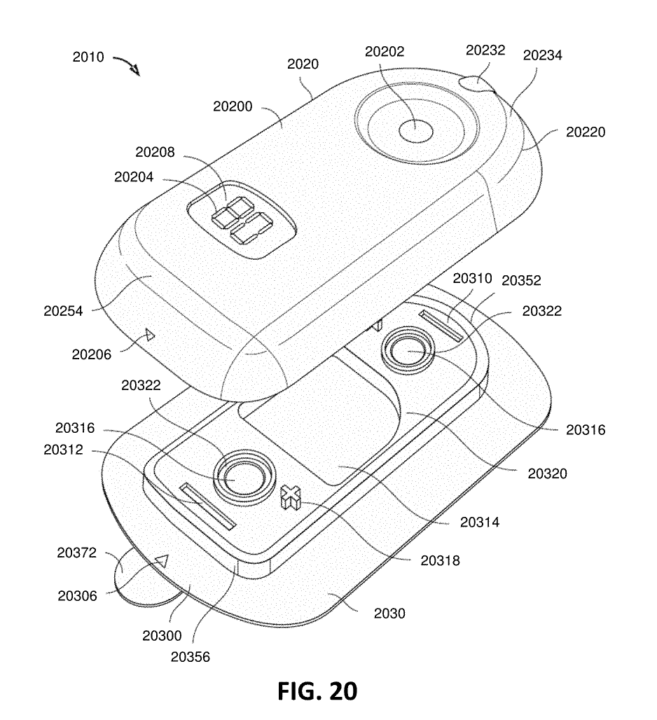

The device described herein may be appreciated by the person skilled in the art upon consideration of the non-limiting examples, which are depicted in the accompanying figures. Starting with FIG. 20, an exemplary electrotransport device 2010 is depicted. The device comprises two parts—an upper part, referred to herein as the electrical module 2020—and a lower part, referred to herein as the reservoir module 2030. The electrical module 2020 includes an electrical module body 20200, which has a top (proximal) surface 20220 and a bottom (distal) surface (not depicted in this view). The module body 20200 has a rounded end 20234 and a squared off end 20254. The top surface 20220 includes a window or aperture 20204 for viewing an LCD display 20208, an activation button 20202 and an LED window or aperture 20232. An alignment feature 20206 is also visible in this view.

The reservoir module 2030 includes a reservoir module body 20300, which supports electrodes, reservoirs (see description herein) and input contacts 20316. In this view, there can be seen upper surface 20320, on which input contact seals 20322, circumscribe the input contacts 20316. The seals 20322 form contaminant-impervious seals with corresponding members on the electrical module 2020 (see description herein). The upper surface 20320 of the reservoir module body 20300 has a rounded end 20352 and a squared off end 20356. Also visible are snap receptors 20310 and 20312, which are configured to cooperate with corresponding snaps on the lower surface of the electrical module 2020. In some embodiments, the snaps 20310 and 20312 are of different dimensions so that each can receive a snap of the correct dimension only, with the result that the device 2010 cannot be assembled in the wrong orientation. As a visual aid to proper alignment of the two modules 2020, 2030, the reservoir module 2030 also has an alignment feature 20306, which a user can align with the alignment feature 20206 on the electrical module 2020 to ensure that the two modules 2020, 2030 are properly aligned.

Also visible in this view is a recess 20314, which in some embodiments is of such a shape as to accept a complementary protruding member on the lower surface of the electrical module 2020 in one orientation only. The recess 20314 and the protuberance on the electrical module 2020 thereby perform a keying function, further ensuring that the two modules can be assembled in one orientation only and/or guiding the user to assemble the two modules in the correct orientation. Another illustrative and non-limiting keying (alignment) feature is the asymmetry of the electrical module 2020 with respect to the reservoir module 2030. As depicted e.g. in FIG. 20, the rounded end 20234 of the electrical module 2020 corresponds to the rounded end 20352 of the reservoir module; and the squared off end 20254 of the electrical module 2020 corresponds to the squared off end 20356 of the reservoir module. The resulting asymmetry helps the user align the electrical module 2020 with the reservoir module 2030 and ensures that user can assemble the two modules in only one orientation. While the rounded end is depicted in this illustration as being distal to the viewer, one of skill in the art will recognize that this is but one possible orientation. As a non-limiting example, the rounded portion may be on the other end or one of the sides of the device. Additional keying features are discussed in more detail herein.

Also depicted in this view is one power-on post 20318, which protrudes from the upper surface 20320 of the reservoir module 2030. The power-on post 20318 is configured to contact a corresponding feature on the electrical module to actuate power-on switches, thereby electrically connecting the battery within the electrical module 2020 into the circuitry contained therein. These features will be described in greater detail below. However, it should be noted that, while there is only one power-on post 20318 depicted in this view, one of the intended power-on posts is obstructed by the perspective of the device. In some embodiments at least two posts and at least two power-on switches are considered advantageous, in that this is considered the minimum number of switches necessary to electrically isolate the battery from the rest of the circuit prior to use. However, this number is merely illustrative and any number of posts and power-on switches may be employed in the devices described herein.

Similarly, while there are two input contacts 20322 depicted, and it is considered necessary that there be at least two such contacts—one positive and one negative—this number is also illustrative only; and any number of contacts—e.g. two positive and one negative, one positive and two negative, two positive and two negative—equal to or greater than two may be employed in devices according to this invention.

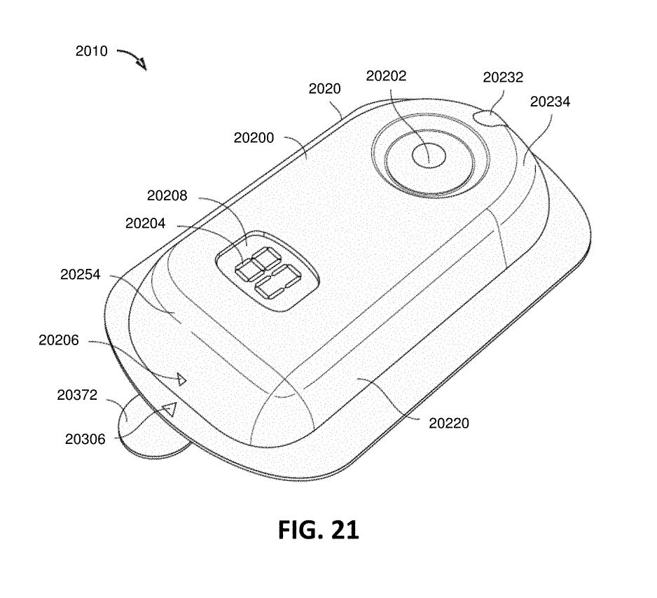

The two modules 2020, 2030 are combined (assembled) prior to use to form the unitary device 2010 depicted in FIG. 21, in which those parts that are visible in FIG. 21 have the same numbers as used in FIG. 20.

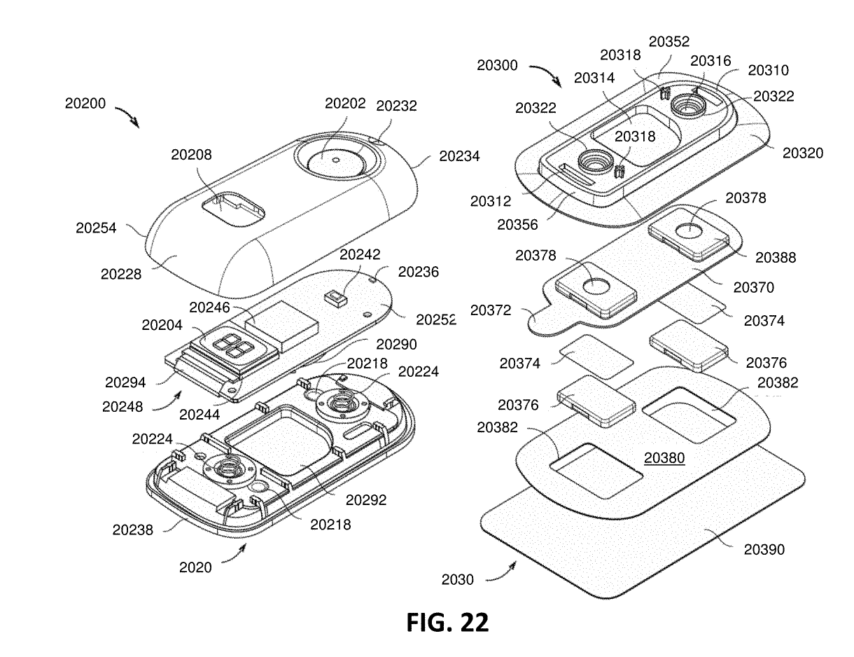

The device 2010 may be further understood by considering FIG. 22, in which the electrical module 20 and the reservoir module 2030 are depicted in exploded perspective views. In the left side of FIG. 22, electrical module 2020 is visible with upper electrical module body 20228, lower electrical module body 20238 and inner electrical module assembly 248. Visible on the upper electrical module body 20228 are the activation button 20202, the LED aperture or window 20232, the LCD aperture or window 20208. While it is also desirable in some embodiments to have an alignment feature on the upper electrical module body 20228, this view does not include such an alignment feature.

Visible on the lower electrical module body 20238 are the upper (proximal) surface of the elastomeric power-on receptacles 20218 as well as springs 20224. The function of the springs 20224 will be described in more detail below. At this point it is noted that the springs 20224 provide bias for connectors on the opposite side of the lower electrical module body 20238.

The electrical circuit assembly 20248 comprises a controller 20244 beneath an LCD display 20204 an LED 20236 and an activation switch 20242, all of which are arranged on a printed circuit board (PCB) 20252. Also barely visible in this exploded view is the battery 20290 on the lower side of PCB 20252. The battery 20290 fits within battery compartment 20292 on the lower electrical module body 20238. A flex circuit 20294, which provides an electrical connection from the PCB 20252 to the LCD display 20204 is also depicted in this view. The LCD display 20204 may be configured to communicate various data to a user, such as a ready indicator, a number of doses administered, a number of doses remaining, time elapsed since initiation of treatment, time remaining in the device's use cycle, battery level, error codes, etc. Likewise the LED 20236 may be used to provide various data to a user, such as indicating that the power is on, the number of doses delivered, etc. The electrical circuit assembly 20248 may also include a sound transducer 20246 which can be configured to provide an audible “power on” signal, an audible “begin dose administration” signal, an audible error alarm, etc.

The reservoir module 2030 appears in exploded perspective view in the right hand side of FIG. 22. The reservoir module 2030 comprises a reservoir body 20300, an electrode housing 20370, an adhesive 20380 and a release liner 20390. The upper surface 20320 of reservoir body 20300 includes the recess 20314, power-on posts 20318, input connectors 20316, seals 20322 and coupler receptacles 20310 and 20312. The electrode housing 20370 includes reservoir compartments 20388. Electrode pads 20374 and reservoirs 20376 are inserted within the reservoir compartments 20388. The electrodes 20374 make contact with the input contacts 20316 through the apertures 20378. The adhesive 20380, which provides means for attaching the device 2010 to a patient, has apertures 20382, through which reservoirs 20376 contact the skin of a patient when the adhesive 20380 is attached to a patient. The removable release liner 20390 covers the reservoirs 20376 and the reservoirs 20376 prior to use, and is removed in order to allow the device 2010 to be attached to a patient. Assembled, the electrode pads 20374 contact the underside of the input connectors 20316 through apertures 20378, providing an electrical connection between the input connectors 20316 and the reservoirs 20376. Connection between the reservoirs 20376 and the patient's skin is made through the apertures 20382 after the release liner 20390 is removed. Also visible in this view is a tab 20372, which can be used to remove the electrode housing 20370 from the reservoir body 20300 for disposal of the reservoirs 20374, which in some embodiments contain residual therapeutic agent, after the device 2010 has been used.

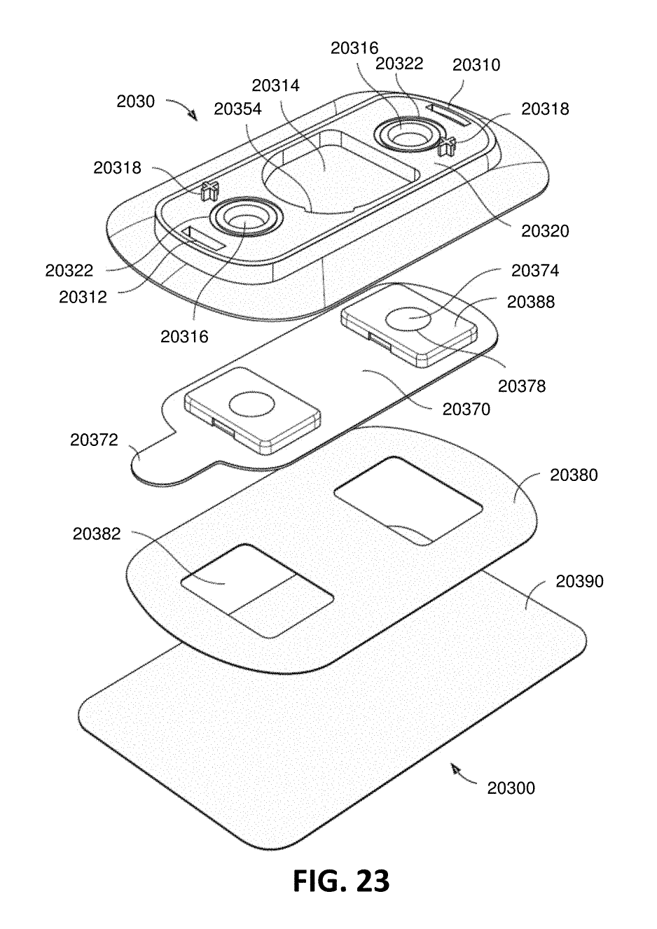

Another view of the reservoir module 2030 appears in FIG. 23. In this view, the electrodes 20374 are viewed through the apertures 20378 in the reservoir compartments 20388. Notable in FIG. 23 is the recess 20314 has an indent 20354, which is adapted to accept a complementary feature on the underside of an electrical module. This is one of many possible keying that may be provided for the device. In some embodiments, the recess 20314 may receive the underside of a battery compartment in the electrical module; however the person skilled in the art will recognize that many such keying features are possible. One such keying feature may be the dimensions of the snap receptacles 20310, 20312 and the corresponding snaps, which permit assembly of the two modules in one configuration only. Other keying features could include the size and/or position of the electrical inputs 20316 on the reservoir module 2030 and the corresponding electrical outputs on the electrical module, the size and/or positions of the power-on posts 20318, the complementary shapes of the reservoir module 2030 and the electrical module 2020.

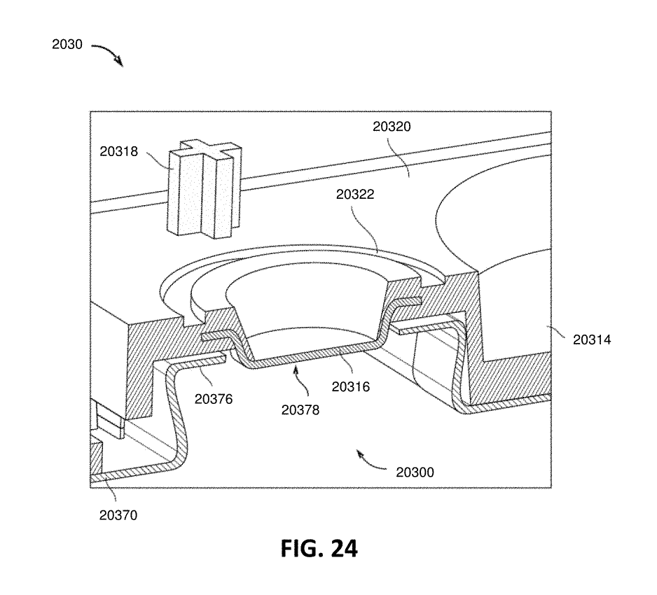

FIG. 24 is a cross section perspective view of an input connector 20316 on a reservoir module 2030. Visible in this view are the upper surface 20320 of the reservoir body 20300. Circumscribing the input connector 20316 is a seal 20322. The seal 20322 is configured to contact a corresponding seal on an electrical module to prevent ingress of contaminants upon assembly of the device. The contact 20316 is in some embodiments advantageously a planar (flat or substantially flat) metallic contact. The contact may be essentially any conductive metal, such as copper, brass, nickel, stainless steel, gold, silver or a combination thereof. In some embodiments, the contact is gold or gold plated.

Also visible on the upper surface 20320 of the reservoir module 2030 is a power-on post 20318 protruding from the surface 20320. The lower portion of input connector 20316 is configured to contact a reservoir (not pictured) through an aperture 20378 in the reservoir compartment 20388 in the electrode housing 20370.

Additionally, part of the battery receptacle 20314 may be seen in FIG. 24.

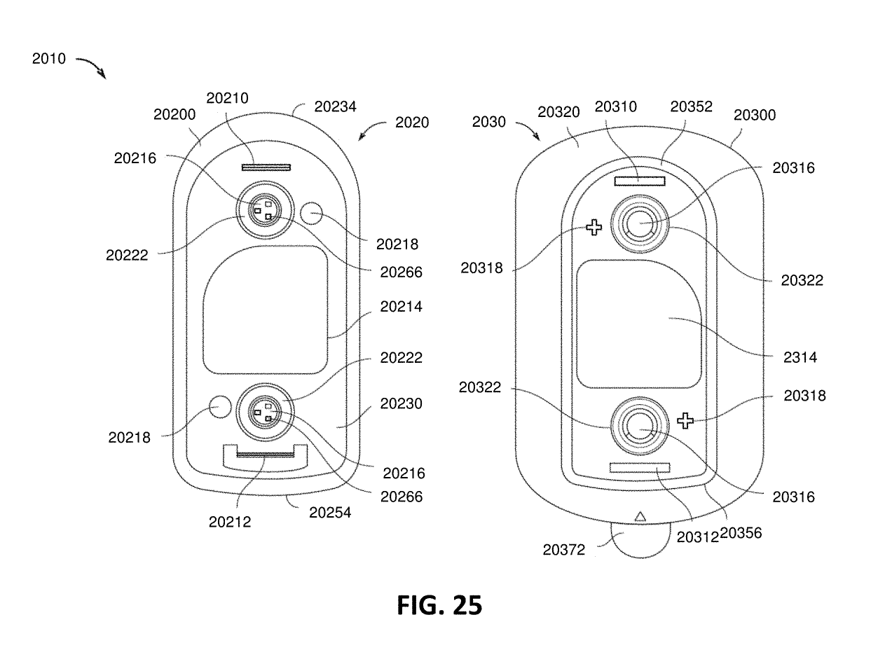

FIG. 25 is another view of the two modules 2020, 2030 side by side. On the left side of FIG. 25 is the bottom side of the electrical module body 20200; and on the right side is the top side of the reservoir module 2030. The bottom surface 20230 of electrical module body 20200 has snaps 20210, 20212 protruding therefrom, which are sized and shaped to fit within the snap receptacles 20310, 20312 on the top of the reservoir module body 20300. As discussed above, in some embodiments snaps 20210 and 20212 are of different size so that snap 20210 will not fit within snap receptacle 20312 and/or snap 20212 will not fit within snap receptacle 20310. This is one of several keying features that may be incorporated in the device 2010. As an illustrative example, snap 20212 cannot fit into 20310, because snap 20212 is larger than receptacle 20310; but snap 20210 can fit into receptacle 20312, because it is the smaller snap an larger receptacle. In other embodiments, it is possible to size both snaps and receptacles so that the one snap/receptacle pair is larger in one dimension (e.g., horizontally), while the other snap/receptacle pair is larger in the other dimension (e.g., longitudinally). Another keying feature is the protrusion 20214, which may house the battery or other component, and which is shaped to fit in one configuration within recess 20314 only.

The snaps 20210, 20212 are at least in some embodiments one-way snaps, meaning that they are biased so as to fit within the receptacles 20310, 20312 in such a way that they are not easily removed, and in at least some preferred embodiments, are configured to break (or deform to the extent that they are no longer operable) if forced apart so that the modules 2020, 2030 cannot be reassembled to form a single unitary device. In some embodiments, such a feature is provided as an anti-abuse character to the device, such that the reservoir module 2030 cannot be saved after use and employed with a different (or the same) electrical module 2020.

The lower surface 20230 of electrical module body 20200 also has two electrical outputs 20216, which are also referred to herein as output “hats”, which in certain embodiments are have one or more bumps 20266 protruding from the surface thereof. These hats 20216 are circumscribed by hat seals 20222. The hats 20216 are configured to make contact with the input connectors 20316 on the reservoir body 20300. Additionally, the hat seals 20222 are configured to contact and create an impermeable seal with the input seals 20322. Advantageously the hat seals 20222 are made of an elastomeric material that creates a contaminant-impermeable seal around the hats 20216 and, when mated with the input connector seals 20322, creates further contaminant-impermeable seals.

The power-on receptacles 20218 are configured to receive input posts 20318. In some embodiments, the power-on receptacles 20218 are made of a deformable (e.g. elastomeric) material. In some such embodiments, the power-on posts 20318 deform the power-on receptacles 20218 so that they contact power-on contacts (described in more detail below) and move them to a closed position, thereby connecting the battery into the circuit. Once the two modules 2020, 2030 are snapped together, the posts maintain pressure on the power-on contacts through the receptacles 20218 and keep the battery in the circuit.

While the hats 20216 and input contacts 20316 are depicted in FIG. 25 as being essentially the same size and symmetrically disposed along the longitudinal axis of the device 2010, another keying feature may be introduced into the device by changing the relative size and/or position with respect to the longitudinal axis of the hats 20216 and contacts 20316, the power-on posts 20318 and receptacles 20218, etc.

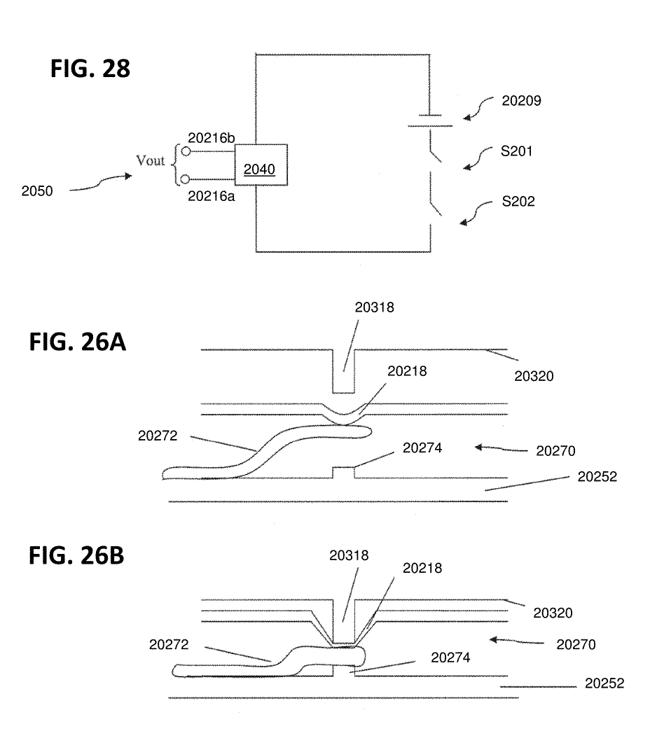

A cross section of one embodiment of a power-on switch 20270 is depicted in FIGS. 26A and 26B. The power-on switch 20270 comprises movable contact 20272 and a stationary contact 20274. Each of the movable contact 20272 and the stationary contact 20274 is connected to a portion of the circuitry on the printed circuit board (PCB) 20252. In the open position depicted in FIG. 26A, the movable contact 20272 is biased away from the stationary contact 20274, whereas in the closed position depicted in FIG. 26B, the two contacts 20272 and 20274 are pressed together by the power-on post 20318, which protrudes from the upper surface 20320 of the reservoir module 2030. The power-on post 20318 acts through the flexible (elastomeric) power-on receptacle 20218 to force the movable contact 20272 down until it is in contact with the stationary contact 20274. For the sake of visibility, the stationary contact 20274 is shown elevated from the PCB 20252; however, it will be understood that the stationary contact 20274 need not be, and generally will not be, elevated from the PCB 20252. In at least some embodiments, the stationary contact 20274 will be an exposed metal trace on the surface of the PCB 20252, though other configurations are also possible. The stationary contact 20272 is manufactured from a suitably springy metal, such as a copper alloy, which is biased to remain in the first, open position unless acted on by the power-on post 20318. The receptacle 20218 may resemble a dome when viewed from the side of facing the contacts 20272, 20274, and is at least in some embodiments formed of a suitable elastomeric substance that permits the power-on post 20318 to deform it without rupturing the seal. In some embodiments, the receptacle 20218 may also be planar or may be domed in the opposite direction. In at least some embodiments, the receptacle 20218 provides a contaminant-tight seal between the external and internal parts of the electrical module 2020.

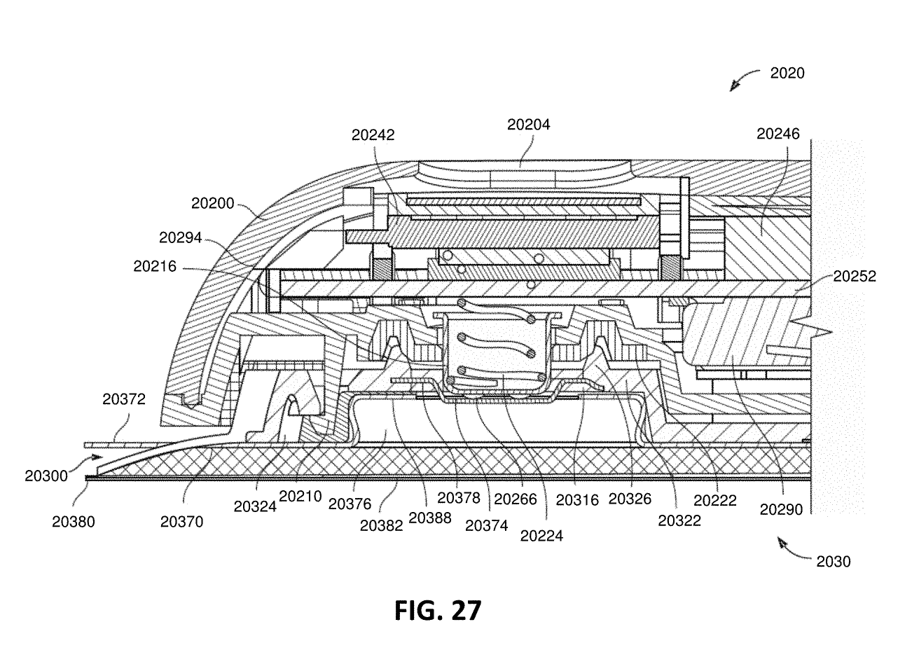

FIG. 27 shows a cross section of a part of a device 2010 in an assembled state. The device 2010 comprises the upper electrical module 2020, comprising an upper body 20200, and the reservoir module 2030, comprising reservoir body 20300, which are shown in this cross section view as combined. Parts of the electrical module 2020 that are visible in this cross section view include the electrical module body 20200, which contains a sound transducer 20246, an LCD 204, controller 242, and battery 290, all of which are on the printed circuit board (PCB) 20252. A flex circuit 20294 provides a connection between the PCB 20252 and the LCD 20204. Also visible are the contact hat 20216, which has bumps 20266, and snap 20210. As can be seen, the contact hat 20216 is biased toward the reservoir module 2030 by a coil spring 20224, which fits within the contact hat 20216 and exerts a force through the contact hat 20216 to press the contact hat 20216 against the input connector 20316 of the reservoir module 2030. The hat 20216 is circumscribed by a hat seal 20222, which contacts the hat 20216 through its full length of travel. In at least some embodiments, this hat seal 20222 is an elastomeric seal that provides a contaminant-tight fit between the hat seal 20222 and the hat 20216, whereby the electrical module 2020 is sealed against contaminants such as particles and fluids (e.g. humidity) in the environment.

The reservoir module 2030 includes a reservoir 20376 and an electrode 20374 within the reservoir compartment 20388 in the electrode housing 20370, which also has an electrode housing tab 20372. In the assembled state, the snap 20210 catches on the ledge 20324 of the snap receptacle 20310. At least in some embodiments, the snap 20210 is made of a resilient polymer and is biased to maintain contact with the ledge 20324 so that the two modules 2020, 2030 cannot be easily separated. In some preferred embodiments, the snap 20210 is configured so that if the two modules 2020, 2030 are separated, the snap 20210 (and/or the ledge 20324) will break (or deform to the extent that they are no longer operable) and thereafter be unable to couple the two modules together.

Also depicted in this view is an input connector seal 20322, which in this illustration forms a ridge 20326 (input connector seal ridge) that circumscribes the input connector 20316. When the two modules 2020, 2030 are assembled, this input connector seal ridge 20326 contacts and presses into the elastomeric hat seal 20222, thereby preventing ingress of contaminants, such as particulates and liquids, into the space containing the output contact hat 20216 and the input contact 20316.

The hat 20216 projects through the aperture 20378 in the reservoir compartments 20388. At least the bumps 20266 on the hat 20216 contact the input connector 20316 to provide electrical contact between the electrical module 2020 and the reservoir module 2030. The spring 20224 provides mechanical bias to force the bumps 20266 to maintain contact with the input connector 20316. Although the hat 20216 is shown being biased by a coil spring 20224, the person having skill in the art will recognize that other springs and spring-like devices can be used within the scope of the device described herein. For example, and without limitation, the coil spring 20224 could be replaced by a beam spring or similar device.

As can be seen in FIG. 28, which is a high level schematic diagram of the electronics 2050 within the electrical module 2020, the electronics 2050 can be envisioned as including circuitry 2040 (which includes the controller, various indicators, etc.) connected to the battery 20290 through power-on switches S201 and S202 (which correspond to power-on switch 20270 in FIGS. 26A, 26B). The circuitry 2040 controls delivery of voltage Vout through the ouputs 20216a, 20216b, which connect to corresponding inputs on the reservoir module. It is to be understood that, although the configuration of power-on switches S201 and S202 shown in FIGS. 26A and 26B is considered to provide certain advantages, such as ease of operation and manufacture, other configurations of switches may be employed within the scope of the device described herein. Such switches may include slides switches that are mechanically biased toward the open position, which may be pushed to the closed position by a power-on post or similar actuator. As can be seen in this figure, the circuit 2050 comprising the battery 20209 and the rest of the circuitry 2040, is only completed if both S201 and S202 are both held closed. Prior to S201 and S202 being closed, e.g. through the mechanical action of power-on posts, the battery 20290 is isolated from the circuitry 2040, as the circuit is open and does not allow current to flow through it. As mentioned before, this reduces battery drain prior to use and greatly reduces corrosion, as the circuitry has no power supply, and thus no extrinsic charge, applied to it. Also, if during handling prior to use one of the switches happens to close, e.g. for a brief period of time, the device will not power on. At least in some embodiments, it is considered advantageous for the controller to detect spurious short-lived closing of both switches S201 and S202 in order to account for occasional, accidental closing of the switches before use. Also, as discussed above, it is considered advantageous in some embodiments that the two switches S201 and S202 be physically and/or electrically remote from one another. Separation of the two switches reduces the likelihood that something that causes one of the switches to malfunction (e.g. close, whether permanently, reversibly or intermittently) will not also affect the other switch. Additionally or alternatively, the two switches may be located on two different sides of the battery or on the same side of the battery. Thus, while in FIG. 28 the switches S201, S202 are depicted on the positive (+) side of the battery 20290, one or both could be located on the other side of the battery. Thus, 1, 2, 3 or more switches may be located on one (positive or negative) side of the battery and 0, 1, 2, 3 or more switches may be located on the other (negative or positive) side of the battery. Physical separation of the two switches may be from 0.1 cm to several cm, and in some embodiments at least 0.5 cm.

Also apparent is FIG. 28 is that the switches S201, S202 are remote from the outputs 20216a, 20216b. Thus, the outputs from the electrical module to the reservoir module are separated from the switches S201, S202. Though in some preferred embodiments the closing of switches S201, S202 occurs as a result of the same action that connects the outputs 20216a, 20216b to the corresponding inputs on the reservoir module, the switches S201, S202 are remote from the outputs 20216a, 20216b. This allows switches S201, S202 to be entirely internal to the electrical module, and in some embodiments to be sealed against ingress of contaminants, such as water (including vapor) and/or particulates.

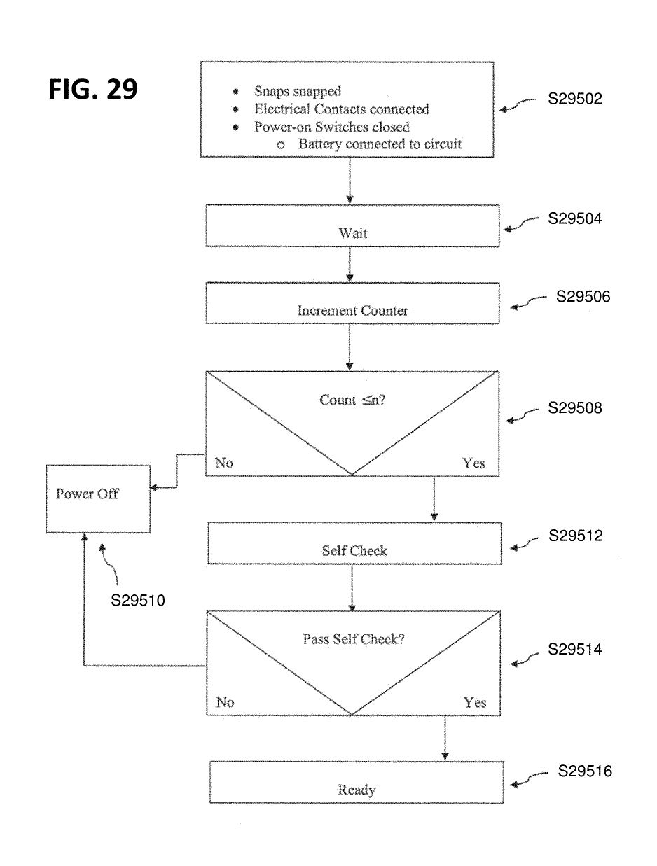

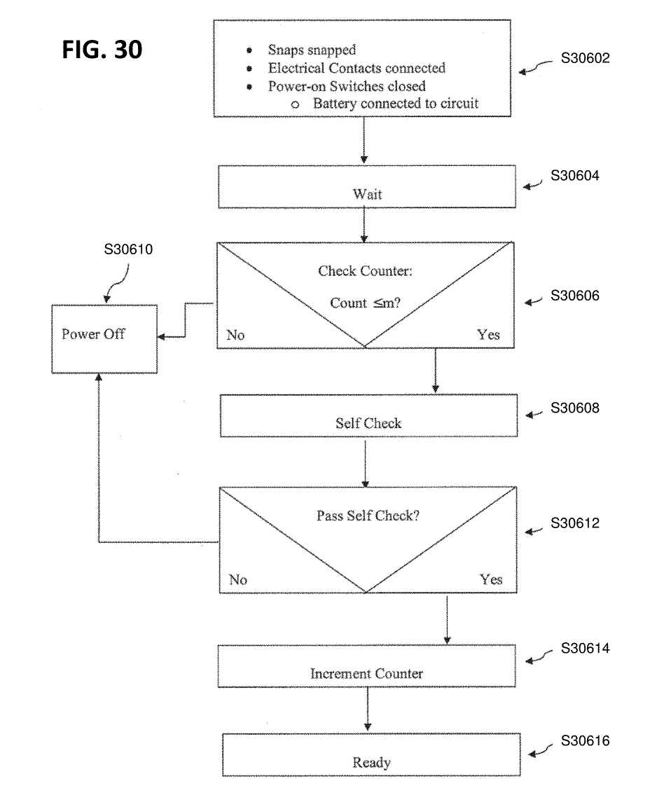

FIGS. 29 and 30 provide two alternative power-on sequences for a device according as described herein. The first alternative in FIG. 29 shows that in the first step, S29502, four events occur all at once in a single action by the user: the snaps are snapped into their respective receptacles; the output and input contacts are mated to provide electrical contact between the reservoirs in the reservoir module and the circuitry in the electrical module; the power-on posts close the power-on switches in the electrical module; and the battery is thereby connected into the circuit and begins providing power to the circuitry. In step S29504 the controller waits a minimum period of time (e.g. 10-500 ms) before proceeding to the next step. In some embodiments, S29504 is eliminated from the power-on sequence. In embodiments in which S29504 is included in the power-on sequence, if the controller fails to maintain power for a predetermined minimum period of time, that is, e.g. power is lost during this timeframe, the timer resets to zero. Presuming that power is maintained through the time period of step S29504, the controller then increments the power-on counter by 1 in step S29506. In step S29508, the controller then checks the number of counts on the power-on counter, and if it is less than or equal to a certain predetermined number (in this example 2, presuming that the counter had been set to 1 by an in-factory test, though other values are possible) the controller proceeds to step S29510, which includes a self check. If, however, the count is greater than the predetermined number, then the controller initiates step S29516, which includes a power off sequence, which may include sending an error message to an LCD display, activating an LED indicator and/or sounding an audible alarm. If the count is less than or equal to the predetermined number, the controller initiates step S29510. After the self check of S29510 is completed, the controller determines whether the circuitry has passed the self check, and if not, it initiates step S29516. If the circuitry passes the self test check, the controller then initiates S29512, which may include signaling the user that the device is ready (e.g. through the LCD, LED and/or sound transducer). The device is then ready to be applied to the body of a patient and operated normally, e.g. as described in U.S. Pat. No. 6,216,033 B1, which is incorporated herein by reference in its entirety.

A second alternative in FIG. 30 shows that in the first step, S30602, four events occur all at once in a single action by the user: the snaps are snapped into their respective receptacles; the output and input contacts are mated to provide electrical contact between the reservoirs in the reservoir module and the circuitry in the electrical module; the power-on posts close the power-on switches in the electrical module; and the battery 20290 is thereby connected into the circuit and begins providing potential to the circuitry. In step S30604 the controller waits a minimum period of time (e.g. 10-500 ms) before proceeding to the next step. If the controller fails to maintain power for this period of time, that is, power is lost during this timeframe, the timer resets to zero. Presuming that power is maintained through the time period of step S30604, the controller then checks the number of counts on the power-on counter in S30606, and if it is less than or equal to a certain predetermined number (in this example 1, presuming that the counter had been set to 1 by an in-factory test, though other values are possible) the controller proceeds to step S30610, which includes a self check. If, however, the count is greater than the predetermined number, then the controller initiates step S30616, which includes a power off sequence, which may include sending an error message to an LCD display, activating an LED indicator and/or sounding an audible alarm. If the count is less than or equal to the predetermined number, the controller initiates step S30610. After the self check of S30610 is completed, the controller determines whether the circuitry has passed the self check, and if not, it initiates step S30616. If the circuitry passes the self test check, the controller then initiates S30612, which includes incrementing the counter by 1. The controller then initiates S30614, which may include signaling the user that the device is ready (e.g. through the LCD, LED and/or sound transducer). The device is then ready to be applied to the body of a patient and operated normally, e.g. as described in U.S. Pat. No. 6,216,033 B1, which is incorporated herein by reference in its entirety.

Briefly described, the device is applied to the surface of a patient's skin. The patient or a healthcare professional may then press the button 20202 (see FIGS. 20, 21, 22). In some embodiments, the device is configured to require the patient or healthcare professional to press the button twice within a predetermined timeframe in order to prevent accidental or spurious administration of the therapeutic agent. Provided the patient or healthcare professional properly presses the button 20202, the device 2010 then begins administering the therapeutic agent to the patient. Once a predetermined number of doses has been administered and/or a predetermined period of time has elapsed since the device was powered on, the device initiates a power off sequence, which may include sending a power off signal to the user through an LCD display, an LED and/or an audio transducer. See especially the claims of U.S. Pat. No. 6,216,033 B1, which are incorporated herein by reference.

The person skilled in the art will recognize that other alternative power-on sequences may be employed. For example, the controller may increment the counter immediately after the counter check in the process outlined in FIG. 29 or 30.

The reservoir of the electrotransport delivery devices generally contain a gel matrix, with the drug solution uniformly dispersed in at least one of the reservoirs. Other types of reservoirs such as membrane confined reservoirs are possible and contemplated. The application of the present invention is not limited by the type of reservoir used. Gel reservoirs are described, e.g., in U.S. Pat. Nos. 6,039,977 and 6,181,963, which are incorporated by reference herein in their entireties. Suitable polymers for the gel matrix can comprise essentially any synthetic and/or naturally occurring polymeric materials suitable for making gels. A polar nature is preferred when the active agent is polar and/or capable of ionization, so as to enhance agent solubility. Optionally, the gel matrix can be water swellable nonionic material.

Examples of suitable synthetic polymers include, but are not limited to, poly(acrylamide), poly(2-hydroxyethyl acrylate), poly(2-hydroxypropyl acrylate), poly(N-vinyl-2-pyrrolidone), poly(n-methylol acrylamide), poly(diacetone acrylamide), poly(2-hydroxylethyl methacrylate), poly(vinyl alcohol) and poly(allyl alcohol). Hydroxyl functional condensation polymers (i.e., polyesters, polycarbonates, polyurethanes) are also examples of suitable polar synthetic polymers. Polar naturally occurring polymers (or derivatives thereof) suitable for use as the gel matrix are exemplified by cellulose ethers, methyl cellulose ethers, cellulose and hydroxylated cellulose, methyl cellulose and hydroxylated methyl cellulose, gums such as guar, locust, karaya, xanthan, gelatin, and derivatives thereof. Ionic polymers can also be used for the matrix provided that the available counterions are either drug ions or other ions that are oppositely charged relative to the active agent.

Incorporation of the drug solution into the gel matrix in a reservoir can be done in any number of ways, i.e., by imbibing the solution into the reservoir matrix, by admixing the drug solution with the matrix material prior to hydrogel formation, or the like. In additional embodiments, the drug reservoir may optionally contain additional components, such as additives, permeation enhancers, stabilizers, dyes, diluents, plasticizer, tackifying agent, pigments, carriers, inert fillers, antioxidants, excipients, gelling agents, anti-irritants, vasoconstrictors and other materials as are generally known to the transdermal art. Such materials can be included by on skilled in the art.

The drug reservoir can be formed of any material as known in the prior art suitable for making drug reservoirs. The reservoir formulation for transdermally delivering cationic drugs by electrotransport is preferably composed of an aqueous solution of a water-soluble salt, such as HCl or citrate salts of a cationic drug, such as fentanyl or sufentanil. More preferably, the aqueous solution is contained within a hydrophilic polymer matrix such as a hydrogel matrix. The drug salt is preferably present in an amount sufficient to deliver an effective dose by electrotransport over a delivery period of up to about 20 minutes, to achieve a systemic effect. The drug salt typically includes about 0.05 to 20 wt % of the donor reservoir formulation (including the weight of the polymeric matrix) on a fully hydrated basis, and more preferably about 0.1 to 10 wt % of the donor reservoir formulation on a fully hydrated basis. In one embodiment the drug reservoir formulation includes at least 30 wt % water during transdermal delivery of the drug. Delivery of fentanyl and sufentanil has been described in U.S. Pat. No. 6,171,294, which is incorporated by reference herein. The parameter such as concentration, rate, current, etc. as described in U.S. Pat. No. 6,171,294 can be similarly employed here, since the electronics and reservoirs of the present invention can be made to be substantially similar to those in U.S. Pat. No. 6,171,294.

The drug reservoir containing hydrogel can suitably be made of any number of materials but preferably is composed of a hydrophilic polymeric material, preferably one that is polar in nature so as to enhance the drug stability. Suitable polar polymers for the hydrogel matrix include a variety of synthetic and naturally occurring polymeric materials. A preferred hydrogel formulation contains a suitable hydrophilic polymer, a buffer, a humectant, a thickener, water and a water soluble drug salt (e.g. HCl salt of an cationic drug). A preferred hydrophilic polymer matrix is polyvinyl alcohol such as a washed and fully hydrolyzed polyvinyl alcohol (PVOH), e.g. MOWIOL 66-100 commercially available from Hoechst Aktiengesellschaft. A suitable buffer is an ion exchange resin which is a copolymer of methacrylic acid and divinylbenzene in both an acid and salt form. One example of such a buffer is a mixture of POLACRILIN (the copolymer of methacrylic acid and divinyl benzene available from Rohm & Haas, Philadelphia, Pa.) and the potassium salt thereof. A mixture of the acid and potassium salt forms of POLACRLIN functions as a polymeric buffer to adjust the pH of the hydrogel to about pH 6. Use of a humectant in the hydrogel formulation is beneficial to inhibit the loss of moisture from the hydrogel. An example of a suitable humectant is guar gum. Thickeners are also beneficial in a hydrogel formulation. For example, a polyvinyl alcohol thickener such as hydroxypropyl methylcellulose (e.g. METHOCEL K100 MP available from Dow Chemical, Midland, Mich.) aids in modifying the rheology of a hot polymer solution as it is dispensed into a mold or cavity. The hydroxypropyl methylcellulose increases in viscosity on cooling and significantly reduces the propensity of a cooled polymer solution to overfill the mold or cavity.

Polyvinyl alcohol hydrogels can be prepared, for example, as described in U.S. Pat. No. 6,039,977. The weight percentage of the polyvinyl alcohol used to prepare gel matrices for the reservoirs of the electrotransport delivery devices, in certain embodiments can be about 10% to about 30%, preferably about 15% to about 25%, and more preferably about 19%. Preferably, for ease of processing and application, the gel matrix has a viscosity of from about 1,000 to about 200,000 poise, preferably from about 5,000 to about 50,000 poise. In certain preferred embodiments, the drug-containing hydrogel formulation includes about 10 to 15 wt % polyvinyl alcohol, 0.1 to 0.4 wt % resin buffer, and about 1 to 30 wt %, preferably 1 to 2 wt % drug. The remainder is water and ingredients such as humectants, thickeners, etc. The polyvinyl alcohol (PVOH)-based hydrogel formulation is prepared by mixing all materials, including the drug, in a single vessel at elevated temperatures of about 90 degree C. to 95 degree C. for at least about 0.5 hour. The hot mix is then poured into foam molds and stored at freezing temperature of about −35 degree C. overnight to cross-link the PVOH. Upon warming to ambient temperature, a tough elastomeric gel is obtained suitable for ionic drug electrotransport.

A variety of drugs can be delivered by electrotransport devices. In certain embodiments, the drug is a narcotic analgesic agent and is preferably selected from the group consisting of fentanyl and related molecules such as remifentanil, sufentanil, alfentanil, lofentanil, carfentanil, trefentanil as well as simple fentanyl derivatives such as alpha-methyl fentanyl, 3-methyl fentanyl and 4-methyl fentanyl, and other compounds presenting narcotic analgesic activity such as alphaprodine, anileridine, benzylmorphine, beta-promedol, bezitramide, buprenorphine, butorphanol, clonitazene, codeine, desomorphine, dextromoramide, dezocine, diampromide, dihydrocodeine, dihydrocodeinone enol acetate, dihydromorphine, dimenoxadol, dimeheptanol, dimethylthiambutene, dioxaphetyl butyrate, dipipanone, eptazocine, ethylmethylthiambutene, ethylmorphine, etonitazene, etorphine, hydrocodone, hydromorphone, hydroxypethidine, isomethadone, ketobemidone, levorphanol, meperidine, meptazinol, metazocine, methadone, methadyl acetate, metopon, morphine, heroin, myrophine, nalbuphine, nicomorphine, norlevorphanol, normorphine, norpipanone, oxycodone, oxymorphone, pentazocine, phenadoxone, phenazocine, phenoperidine, piminodine, piritramide, proheptazine, promedol, properidine, propiram, propoxyphene, and tilidine.

Some ionic drugs are polypeptides, proteins, hormones, or derivatives, analogs, mimics thereof. For example, insulin or mimics are ionic drugs that can be driven by electrical force in electrotransport.

For more effective delivery by electrotransport salts of certain pharmaceutical analgesic agents are preferably included in the drug reservoir. Suitable salts of cationic drugs, such as narcotic analgesic agents, include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, .beta.-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate. The more preferred salt is chloride.

A counterion is present in the drug reservoir in amounts necessary to neutralize the positive charge present on the cationic drug, e.g. narcotic analgesic agent, at the pH of the formulation. Excess of counterion (as the free acid or as a salt) can be added to the reservoir in order to control pH and to provide adequate buffering capacity. In one embodiment of the invention, the drug reservoir includes at least one buffer for controlling the pH in the drug reservoir. Suitable buffering systems are known in the art.

The device described herein is also applicable where the drug is an anionic drug. In this case, the drug is held in the cathodic reservoir (the negative pole) and the anoidic reservoir would hold the counterion. A number of drugs are anionic, such as cromolyn (antiasthmatic), indomethacin (anti-inflammatory), ketoprofen (anti-inflammatory) and ketorolac tromethamine (NSAID and analgesic activity), and certain biologics such as certain protein or polypeptides.

### Method of Making

A device according to the present invention can be made by forming the layers separately and assembling the layers into the electronic module and the reservoir module. The polymeric layers can be made by molding. Some of the layers can be applied together and secured. Some of the layers can be comolded, for example, by molding a second layer onto a first layer. For example, the upper layer and lower layer of the upper cover (or top cover) can be comolded together. Some of the layers can be affixed together by adhesive bonding or mechanical anchoring. Such chemical adhesive bonding methods and mechanical anchoring methods are known in the art. As described before, once the electronic module and the reservoir module are formed, they can be packaged separately. Before use, the two modules can be removed from their respective packages and assembled to form the device for electrotransport. The device can then be applied to the body surface by adhesion.

The above-described exemplary embodiments are intended to be illustrative in all respects, rather than restrictive, of the present invention. Thus the present invention is capable of many variations in detailed implementation that can be derived from the description contained herein by a person skilled in the art, e.g., by permutation or combination of various features. Although iontophoretic devices are described in detail as illustration for showing how an electronic module and an agent module are coupled and work together, a person skilled in the art will know that electronic module and agent module in other electrotransport devices can be similarly coupled and work together. All such variations and modifications are considered to be within the scope of the present invention. The entire disclosure of each patent, patent application, and publication cited or described in this document is hereby incorporated herein by reference.

While preferred embodiments of the present invention have been shown and described herein, those skilled in the art will recognize that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

One method for transdermal delivery of active agents involves the use of electrical current to actively transport the active agent into the body through intact skin by electrotransport. Electrotransport techniques may include iontophoresis, electroosmosis, and electroporation. Electrotransport devices, such as iontophoretic devices are known in the art. One electrode, which may be referred to as the active or donor electrode, is the electrode from which the active agent is delivered into the body. The other electrode, which may be referred to as the counter or return electrode, serves to close the electrical circuit through the body. In conjunction with the patient's body tissue, e.g., skin, the circuit is completed by connection of the electrodes to a source of electrical energy, and usually to circuitry capable of controlling the current passing through the device when the device is “on” and delivering current. If the substance to be driven into the body is ionic and is positively charged, then the positive electrode (the anode) will be the active electrode and the negative electrode (the cathode) will serve as the counter electrode. If the ionic substance to be delivered is negatively charged, then the cathodic electrode will be the active electrode and the anodic electrode will be the counter electrode.

A switch-operated therapeutic agent delivery device can provide single or multiple doses of a therapeutic agent to a patient by activating a switch. Upon activation, such a device delivers a therapeutic agent to a patient. A patient-controlled device offers the patient the ability to self-administer a therapeutic agent as the need arises. For example, the therapeutic agent can be an analgesic agent that a patient can administer whenever sufficient pain is felt.

As described in greater detail below, any appropriate drug (or drugs) may be delivered by the devices described herein. For example, the drug may be an analgesic such as fentanyl (e.g., fentanyl HCL) or sufantanil.

In some variations, the different parts of the electrotransport system are stored separately and connected together for use. For example, examples of electrotransport devices having parts being connected together before use include those described in U.S. Pat. No. 5,320,597 (Sage, Jr. et al); U.S. Pat. No. 4,731,926 (Sibalis), U.S. Pat. No. 5,358,483 (Sibalis), U.S. Pat. No. 5,135,479 (Sibalis et al.), UK Patent Publication GB2239803 (Devane et al), U.S. Pat. No. 5,919,155 (Lattin et al.), U.S. Pat. No. 5,445,609 (Lattin et al.), U.S. Pat. No. 5,603,693 (Frenkel et al.), WO1996036394 (Lattin et al.), and U.S. 2008/0234628 A1 (Dent et al.).

In general, the systems and devices described herein include an anode and cathode for the electrotransport of a drug or drugs into the patient (e.g., through the skin or other membrane) and a controller for controlling the delivery (e.g., turning the delivery on or off); all of the variations described herein may also include an off-current module for monitoring the anode and cathode when the activation circuit is in the off state while still powered on to determine if there is a potential and/or current (above a threshold value) between the anode and cathode when the controller for device has otherwise turned the device “off” so that it should not be delivering drug to the patient. The controller may include an activation controller (e.g., an activation module or activation circuitry) for regulating the when the device is on, applying current/voltage between the anode and cathode and thereby delivering drug.

Throughout this specification, unless otherwise indicated, singular forms “a”, “an” and “the” are intended to include plural referents. Thus, for example, reference to “a polymer” includes a single polymer as well as a mixture of two or more different polymers, “a contact” may refer to plural contacts, “a post” may indicate plural posts, etc.

As used herein, the term “user” indicates anyone who uses the device, whether a healthcare professional, a patient, or other individual, with the aim of delivering a therapeutic agent to a patient.

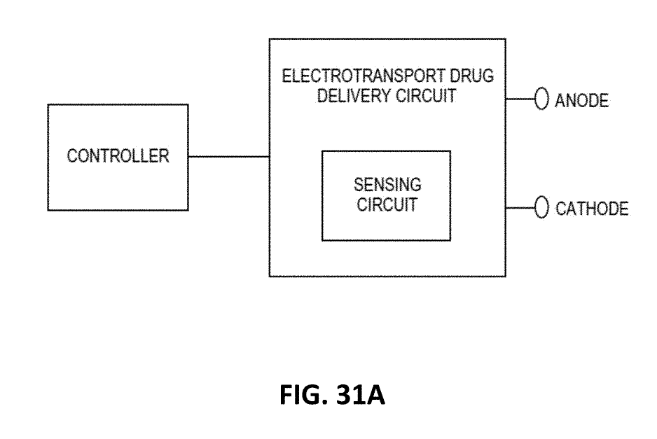

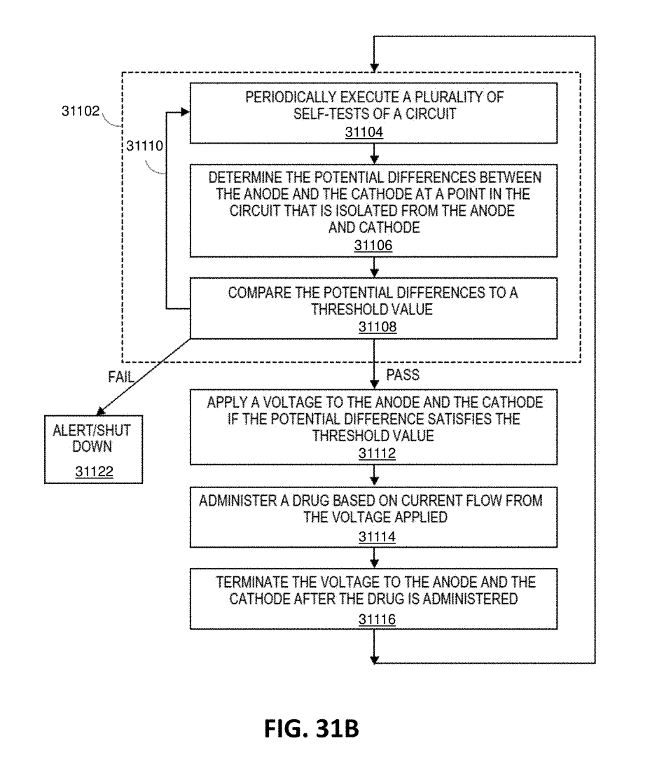

In general, the off-current module may include hardware, software, firmware, or some combination thereof (including control logic). For example, as illustrated in FIG. 31A, a system may include an anode, cathode and sensing circuit. The sensing circuit may form part (or be used by) the off-current module to sense any current between the anode and cathode when the device is otherwise off. The device may also include a controller controlling operation of the device. The controller may include a processor or ASIC that includes the off-current module.

In general, and off-current module may also be referred to as a type of self-test that is performed by the device. In some variations, the off-current module includes or is referred to as an anode/cathode voltage difference test or off-current test, because in some variations it may determine if there is a voltage difference between the anode and cathode when the device should be off.

FIG. 31B illustrates a simplified version of one method of performing an anode/cathode voltage difference test (also referred to as an off-current test). Initially, when the device is powered on but it is not activated to deliver drug (e.g., is in powered on but in an off state), the device may periodically perform any number of self-tests while in this “ready” mode. In particular, the device may perform the off-current test to confirm that while the device is otherwise off, there is not a significant current flowing (which may be inferred, e.g., by determining that there isn't a potential difference above a threshold level sufficient to deliver drug to the patient) between the anode and cathode. In embodiments in which the current is determined by monitoring potential difference, this potential difference may readily be determined by examining the difference between the voltage at the anode and the voltage at the cathode. Any other subsystem or method of measuring and/or inferring current flow between the anode and cathode may also be used as long as the testing method itself does not result in undesirable drug delivery.

Returning to FIG. 31B, in the initial step 31102 the self-test(s) such as the off-current self-test may be periodically and automatically performed while the device is in the ready mode. The off-current self-test may be timed and executed by control logic (e.g., executing on a controller) which may be part of another controller or may be a controller. In general the controller (or portion of a controller) performing the off-current test may be referred to as an off-current module. The self-test may be triggered at regular intervals, such as every 30 seconds, every minute, every two minutes, etc. Once the self-test is triggered, in some variations it may be performed by determining the difference between the voltage at the anode and the voltage at the cathode in a manner that does not trigger release of drug. For example, the determination of the voltage of the anode may be isolated from the determination of the voltage of the cathode 31104. The difference in the voltages may next be compared to a threshold value 31106, which may be referred to as the off-current threshold. Examples of this threshold value include 0.5V, 0.75V, 0.85V, 2.5V, etc. If the difference is less than the threshold value than the device “passes” the self-test, and may continue in “ready” mode 31110, or, if the activation of the device has been triggered (e.g., by pressing button), the device my begin delivering drug 31112-31116. Alternatively, if a leak current is detected, e.g., when the voltage difference is greater than (or equal to) the threshold voltage (fail 31122), the device may trigger an alert and/or may shut down to prevent unwanted delivery of drug.

### Example 1: Two-Part System

Described below is one example of a two part system that may include self-tests including in particular an anode/cathode voltage difference test. For example, in some variations the devices including the off-current self-test are configured as two-part electrotransport therapeutic agent delivery devices, such as iontophoresis devices, in which the two parts of the device are provided separately and assembled to form a unitary, powered-on device at the point of use—that is to say just prior to use. In this example, one part of the device, which may be referred to herein as the electrical module, holds essentially all of the circuitry, as well as the power source (e.g. battery), for the device; and the other part, which may be referred to herein as the reservoir module, contains the therapeutic agent to be delivered along with electrodes and hydrogels necessary to deliver the therapeutic agent to a patient. The device is configured such that the power source is kept electrically isolated from the rest of the circuitry in the electrical module until the electrical module is combined with the reservoir module. Thus, embodiments provided herein permit the combination of the electrical module and the reservoir module, whereby in a single action the two modules form a single unit and the battery is introduced into the circuitry, thereby powering on the device, in a single action by the user.

As used herein, the term simultaneous, and grammatical variants thereof, indicates that two or more events occur at about the same time and/or that they occur without any intervening step. For example, when connection of the modules occurs simultaneously with connection of the battery into the circuit, the term “simultaneously” indicates that when the modules are connected, the battery is connected into the circuit at about the same time, in a single action by the user, and that there is no additional step necessary on the part of the user to connect the battery to the circuit. The term “substantially simultaneous” and grammatical variants indicates that two events occur at about the same time and no significant action is required by the user between the two events. For the sake of illustration only, such a significant action could be the activation of a separate switch (other than the herein-described power-on switches), removal of a tab, or other action to connect the battery in the electrical module to the circuitry therein upon connection of the two modules to one another.

Unless otherwise modified herein, the term “to break” and grammatical variants thereof refers to destroying or deforming something to the point that it is no longer operable for its intended purpose.

An electrotransport device may be assembled before use for electrotransport delivery of ionic compounds (e.g., ionic drugs such as fentanyl and analogs, polypeptides, and the like) through a surface, such as skin. An electrotransport device may comprise a top or upper portion, herein referred to as an electrical module, and a bottom or lower portion, herein referred to as a reservoir module. The electrical module may contain circuitry (e.g. a printed circuit board), a power source (e.g. a battery), one or more power-on switches and such other circuitry as may be deemed desirable for operation of the device (such as an activation switch, a controller, a liquid crystal diode (LCD) display, a connector, a light emitting diode (LED), an audible indicator (e.g. a sound transducer), or combinations thereof), as well as electrical output contacts for electrically connecting the electrical module to a reservoir module. When obtained by the user, the electrical module is separated from the reservoir module. In this state, the battery is maintained outside of the electrical circuit (though within the electrical module), thereby preventing the battery from discharging through the circuit prior to use. Because the battery is electrically isolated from the circuit prior to combining the electrical and reservoir modules, the circuitry has essentially no electrical charge applied to it prior to combination of the two modules, rendering the circuitry far less susceptible to corrosion than if the battery were in the circuit. In some variations the off-current module may be configured to operate even when the two parts of the device/system are not connected (e.g., even with the device powered off, and/or with the battery driving drug delivery disconnected). Thus, a separate power source/batter may power the off-current module in some variations. In other variations the off-current module may be configured to operate when the device is in an off state, but otherwise powered on (e.g., when the two halves of the system/device are connected). In any of the variations described herein the off-current module may be electrically isolated from the drug delivery sub-components of the device/system. Thus, even if a short occurs in the drug delivery component of the device, the off-current module may operate.

The reservoir module may contain electrodes and reservoirs for delivery of therapeutic agent to a patient. At least one reservoir may contain the therapeutic agent to be delivered. At least one counter reservoir is provided, which generally contains no therapeutic agent, though in some embodiments it is possible for the counter reservoir to contain therapeutic agent. Prior to being connected to the electrical module, the reservoir module is maintained both physically and electrically isolated from the electrical module. For example, one or both of the modules may be sealed in a pouch, such as a plastic or foil pouch, in order to prevent contamination with water, particulates, vapors, etc. As a non-limiting example, both the electrical and the reservoir modules may be sealed in the same pouch. As a further non-limiting example, the reservoir module may be sealed in a pouch and the electrical module left outside the sealed pouch. In other non-limiting examples, the two modules may be sealed in separate pouches.

Prior to use (e.g. just prior to use) the electrical module is combined with the reservoir module to form a single unit, which in a single action, connects the battery into the circuit and powers the device on. The terms “prior to use” and “just prior to use” are described in more detail hereinafter. In general, these terms are intended to indicate that the two parts of the device are combined by a user, and that the device is then used to deliver therapeutic agent to a patient within a predetermined window of time—e.g. from 0 to 8 hrs. or from 0 to 72 hours—after the two parts of the device are combined. This predetermined window of time may vary, depending upon the therapeutic agent, the amount of agent to be delivered, requirements of various regulatory agencies, etc. For the sake of clarity, it is to be understood that combination of the electrical and reservoir modules is postponed after manufacture and is carried out at the point of use so that during shipping and storage the power source enclosed within the electrical module is electrically isolated from the circuitry until the two modules are combined by the user.

As stated before, combination of the electrical and reservoir modules connects the battery into the circuit to achieve a powered on state, without any additional action required on the part of the user. For example, there is no need for the user to activate a power switch or remove a tab in order to connect the battery into the circuit. Once the two modules have been properly combined, power is supplied to the circuitry. The circuitry can then operate normally. Normal operation may include various circuitry tests, operation of various indicators (such as the aforementioned LCD, LED and sound transducers), setting of various logic flags, detection of error states and/or logic flags, etc. Normal operation also includes reception of an activation signal, e.g. through an activation button or switch, and providing power to the electrodes through electrical outputs connected to electrical inputs on the reservoir module.

In addition to reducing corrosion and battery discharge prior to use, another advantage of the device is that the electrical outputs from the electrical module and inputs to the reservoir module (i.e. the contacts between the two modules) are electrically and physically separated from the power-on switches that connect the battery into the circuit. This is advantageous, at least because it allows the power-on switches, which connect the battery into the circuit, to be kept entirely internal to the electrical module. This in turn allows the contacts that comprise the power-on switches to be kept contaminant-free, as the electrical module is at least in some embodiments sealed against contaminants, such as water (including water vapor) and/or particulates. As described herein, a power-on switch is closed by an actuator through an elastomeric seal, which permits the battery to be connected into the circuit without the contacts that comprise the switch being exposed to the environment external to the electrical module.

In some embodiments, two or more power-on switches are employed. In some particular embodiments, the power-on switches are physically remote from one another—e.g. on the order of from 0.1 cm to several cm. In some embodiments, the switches are at least 0.5 cm from one another.

As the two modules form a unitary device, they advantageously include one or more mechanical coupler pairs to hold the two modules together. Such coupler pairs can include snap-snap receptacle pairs, which are in some embodiments designed to become inoperative (deform and/or break) if the two modules are forced apart after they are combined. Thus, devices described herein are well-suited for one time use, as they can be adapted to embody mechanical means for ensuring that the device is used only once.

In some embodiments, the device may alternatively, or additionally, employ electrical means for ensuring that the device is used only once. For example, an electrical means may employ a controller in the electrical module which increments a power-on counter when the device is powered on. In such embodiments, before or after the controller increments the counter, it detects the number of counts on the counter, and if it finds that the power-on counts exceed some predetermined value, it executes a routine to power the device off. As a non-limiting example the counter may initially be set to zero upon manufacturing. The device may then be briefly powered on by an external power supply during post-manufacturing testing, which the controller interprets as one power-on event, and thus increments the power-on counter by 1 count. Then when the device is assembled by the user prior to use, the controller interprets the connection of the battery into the circuit as a power-on event, and increments the power-on counter by 1. The controller then detects the count on the counter. If the count is 2 or less, the controller permits the device to operate normally. If however, the count is 3 or more, the controller initiates a power-off sequence.

As a second, non-limiting example, the counter may initially be set to zero upon manufacturing. The device may then be briefly powered on by an external power supply during post-manufacturing testing, which the controller interprets as one power-on event, and thus increments the power-on counter by 1 count. Then when the device is assembled by the user prior to use, the controller detects the count on the counter. If the count is 1 or less, the controller increments the power-on counter and permits the device to operate normally. If however, the count is 2 or more, the controller initiates a power-off sequence.

Although reference is made here to counting power-on sequences, other events may be counted, either in place of power-on events, in addition to power-on events, or as a proxy for power-on events.

The power off sequence can be a sequence such as described in U.S. Pat. No. 6,216,003 B1, which is incorporated herein in its entirety.

In some embodiments, the device combines both mechanical (e.g. one-way snaps) and electrical (e.g. power-on counter) means to ensure that the device cannot be used more than once.

A single use device/system may include multiple administrations of a therapeutic agent, e.g. within a particular window of time after the device has been powered on. The duration of time during which therapeutic agent may be administered and/or the number of total doses permitted to be administered by the device may be predetermined and programmed into a controller. Means for controlling the number of doses that may be administered and/or the period during which therapeutic may be administered are described e.g. in U.S. Pat. No. 6,216,003 B1, which is incorporated herein in its entirety. For the sake of clarity, the term “single use” is not intended to limit the device to a single administration of drug. Rather, the term “single use” is intended to exclude use of the device on more than one patient or on more than one occasion; it is also intended to exclude the use of an electrical module with more than one reservoir module and/or the reservoir module with more than one electrical module and/or detachment of the reservoir module from the electrical module and reattachment. Thus, single use feature is in some embodiments employed to prevent the patient or another from saving drug and using it at a later time. In some embodiments, such a feature may be employed to prevent abuse of the therapeutic agent.

In at least some embodiments of the device described herein, the device is configured to prevent contamination of the circuitry before and during use in order to reduce the likelihood of device malfunction. For example, the use environment may include emergency room, operative, post-operative or other medical treatment environments, in which potential particulate and liquid are prevalent. Accordingly, at least some embodiments of the device are configured so that one or more seals are formed in order to exclude ambient contaminants from ingress into the working parts of the device, such as in particular the circuitry. In some embodiments, one or more seals are formed around electrical contacts between the electrical outputs on the electrical module and the electrical inputs on the reservoir module.

In some embodiments, the power-on contacts are sealed from ingress of contaminants, such as particulates and fluids. In particular embodiments, the power-on contacts are sealed before the modules are combined, during the act of combination, and after the two modules are combined. In at least some such cases, the power-on contacts may be actuated (switched to a closed position) by an actuator that acts through an interposed elastomer, which maintains an impermeable seal while at the same time being deformed by an actuator (such as a post or other elongate member) to press the power-on contact into a closed position.

Other seals are possible and may be desirable. For example, a seal may be formed between the two parts (modules) when they are combined.

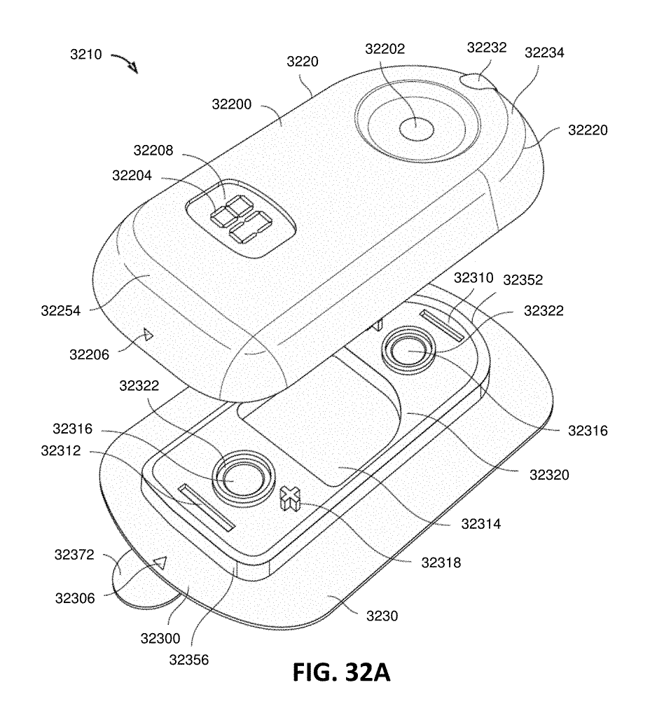

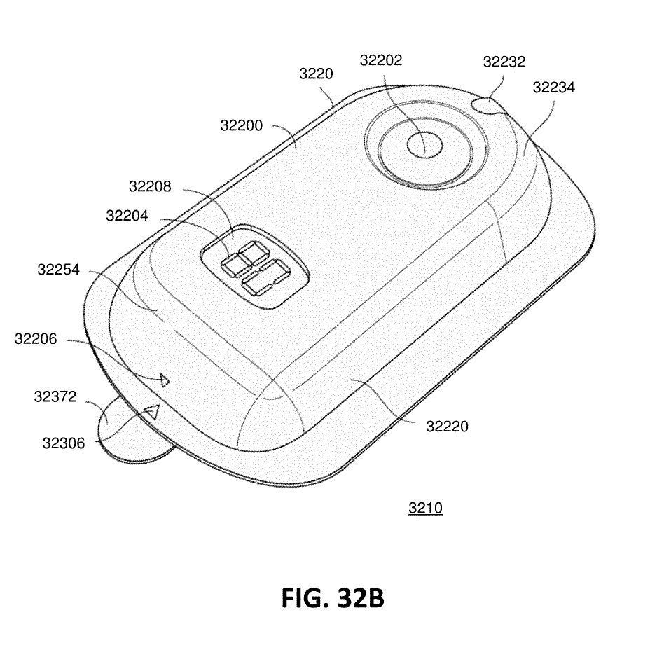

The device described herein may be appreciated by the person skilled in the art upon consideration of the non-limiting examples, which are depicted in the accompanying figures. Starting with FIG. 32A, an exemplary electrotransport device 3210 is depicted. The device comprises two parts—an upper part, referred to herein as the electrical module 3220—and a lower part, referred to herein as the reservoir module 3230. The electrical module 3220 includes an electrical module body 32200, which has a top (proximal) surface 32220 and a bottom (distal) surface (not depicted in this view). The module body 32200 has a rounded end 32234 and a squared off end 32254. The top surface 32220 includes a window or aperture 32204 for viewing an LCD display 32208, an activation button 32202 and an LED window or aperture 32232. An alignment feature 32206 is also visible in this view.

The reservoir module 3230 includes a reservoir module body 32300, which supports electrodes, reservoirs (see description herein) and input contacts 32316. In this view, there can be seen upper surface 32320, on which input contact seals 32322, circumscribe the input contacts 32316. The seals 32322 form contaminant-impervious seals with corresponding members on the electrical module 3220 (see description herein). The upper surface 32320 of the reservoir module body 32300 has a rounded end 32352 and a squared off end 32356. Also visible are snap receptors 32310 and 32312, which are configured to cooperate with corresponding snaps on the lower surface of the electrical module 3220. In some embodiments, the snaps 32310 and 32312 are of different dimensions so that each can receive a snap of the correct dimension only, with the result that the device 3210 cannot be assembled in the wrong orientation. As a visual aid to proper alignment of the two modules 3220, 3230, the reservoir module 3230 also has an alignment feature 32306, which a user can align with the alignment feature 32206 on the electrical module 3220 to ensure that the two modules 3220, 3230 are properly aligned.

Also visible in this view is a recess 32314, which in some embodiments is of such a shape as to accept a complementary protruding member on the lower surface of the electrical module 3220 in one orientation only. The recess 32314 and the protuberance on the electrical module 3220 thereby perform a keying function, further ensuring that the two modules can be assembled in one orientation only and/or guiding the user to assemble the two modules in the correct orientation. Another illustrative and non-limiting keying (alignment) feature is the asymmetry of the electrical module 3220 with respect to the reservoir module 3230. As depicted e.g. in FIG. 32A, the rounded end 32234 of the electrical module 3220 corresponds to the rounded end 32352 of the reservoir module; and the squared off end 32254 of the electrical module 3220 corresponds to the squared off end 32356 of the reservoir module. The resulting asymmetry helps the user align the electrical module 3220 with the reservoir module 3230 and ensures that user can assemble the two modules in only one orientation. While the rounded end is depicted in this illustration as being distal to the viewer, one of skill in the art will recognize that this is but one possible orientation. As a non-limiting example, the rounded portion may be on the other end or one of the sides of the device. Additional keying features are discussed in more detail herein.

Also depicted in this view is one power-on post 32318, which protrudes from the upper surface 32320 of the reservoir module 3230. The power-on post 32318 is configured to contact a corresponding feature on the electrical module to actuate power-on switches, thereby electrically connecting the battery within the electrical module 3220 into the circuitry contained therein. These features will be described in greater detail below. However, it should be noted that, while there is only one power-on post 32318 depicted in this view, one of the intended power-on posts is obstructed by the perspective of the device. In some embodiments at least two posts and at least two power-on switches are considered advantageous, in that this is considered the minimum number of switches necessary to electrically isolate the battery from the rest of the circuit prior to use. However, this number is merely illustrative and any number of posts and power-on switches may be employed in the devices described herein.

Similarly, while there are two input contacts 32322 depicted, and it is considered necessary that there be at least two such contacts—one positive and one negative—this number is also illustrative only; and any number of contacts—e.g. two positive and one negative, one positive and two negative, two positive and two negative—equal to or greater than two may be employed in devices according to this invention.

The two modules 3220, 3230 are combined (assembled) prior to use to form the unitary device 3210 depicted in FIG. 32B, in which those parts that are visible in FIG. 32B have the same numbers as used in FIG. 32A.

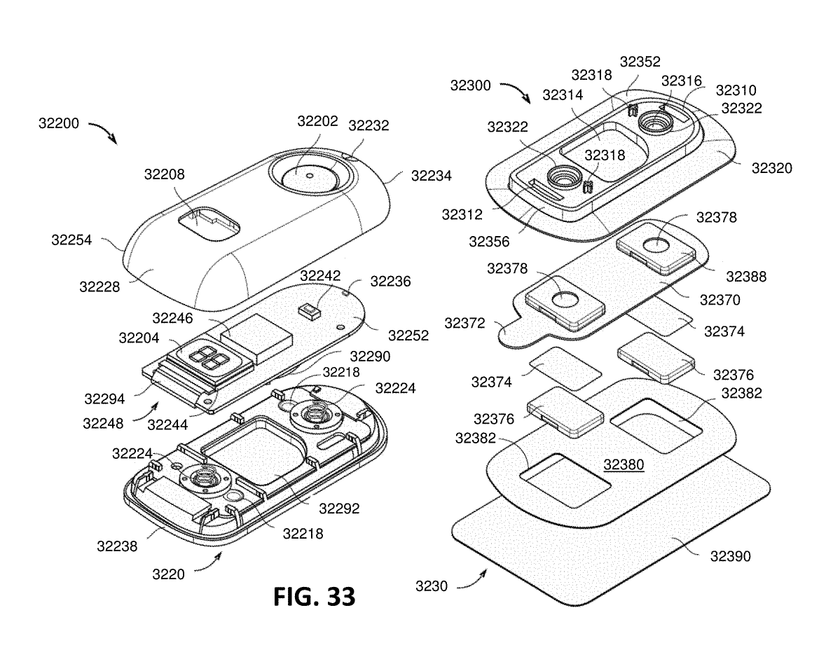

The device 3210 may be further understood by considering FIG. 33, in which the electrical module 3220 and the reservoir module 3230 are depicted in exploded perspective views. In the left side of FIG. 33, electrical module 3220 is visible with upper electrical module body 32228, lower electrical module body 32238 and inner electrical module assembly 32248. Visible on the upper electrical module body 32228 are the activation button 32202, the LED aperture or window 32232, the LCD aperture or window 32208. While it is also desirable in some embodiments to have an alignment feature on the upper electrical module body 32228, this view does not include such an alignment feature.

Visible on the lower electrical module body 32238 are the upper (proximal) surface of the elastomeric power-on receptacles 32218 as well as springs 32224. The function of the springs 32224 will be described in more detail below. At this point it is noted that the springs 32224 provide bias for connectors on the opposite side of the lower electrical module body 32238.

The electrical circuit assembly 32248 comprises a controller 32244 beneath an LCD display 32204 an LED 32236 and an activation switch 32242, all of which are arranged on a printed circuit board (PCB) 32252. Also barely visible in this exploded view is the battery 32290 on the lower side of PCB 32252. The battery 32290 fits within battery compartment 32292 on the lower electrical module body 32238. A flex circuit 32294, which provides an electrical connection from the PCB 32252 to the LCD display 32204, is also depicted in this view. The LCD display 32204 may be configured to communicate various data to a user, such as a ready indicator, a number of doses administered, a number of doses remaining, time elapsed since initiation of treatment, time remaining in the device's use cycle, battery level, error codes, etc. Likewise the LED 32236 may be used to provide various data to a user, such as indicating that the power is on, the number of doses delivered, etc. The electrical circuit assembly 32248 may also include a sound transducer 32246 which can be configured to provide an audible “power on” signal, an audible “begin dose administration” signal, an audible error alarm, etc.

The reservoir module 3230 appears in exploded perspective view in the right hand side of FIG. 33. The reservoir module 3230 comprises a reservoir body 32300, an electrode housing 32370, an adhesive 32380 and a release liner 32390. The upper surface 32320 of reservoir body 32300 includes the recess 32314, power-on posts 32318, input connectors 32316, seals 32322 and coupler receptacles 32310 and 32312. The electrode housing 32370 includes reservoir compartments 32388. Electrode pads 32374 and reservoirs 32376 are inserted within the reservoir compartments 32388. The electrodes 32374 make contact with the input contacts 32316 through the apertures 32378. The adhesive 32380, which provides means for attaching the device 3210 to a patient, has apertures 32382, through which reservoirs 32376 contact the skin of a patient when the adhesive 32380 is attached to a patient. The removable release liner 32390 covers the reservoirs 32376 and the reservoirs 32376 prior to use, and is removed in order to allow the device 3210 to be attached to a patient. Assembled, the electrode pads 32374 contact the underside of the input connectors 32316 through apertures 32378, providing an electrical connection between the input connectors 32316 and the reservoirs 32376. Connection between the reservoirs 32376 and the patient's skin is made through the apertures 32382 after the release liner 32390 is removed. Also visible in this view is a tab 32372, which can be used to remove the electrode housing 32370 from the reservoir body 32300 for disposal of the reservoirs 32374, which in some embodiments contain residual therapeutic agent, after the device 3210 has been used.

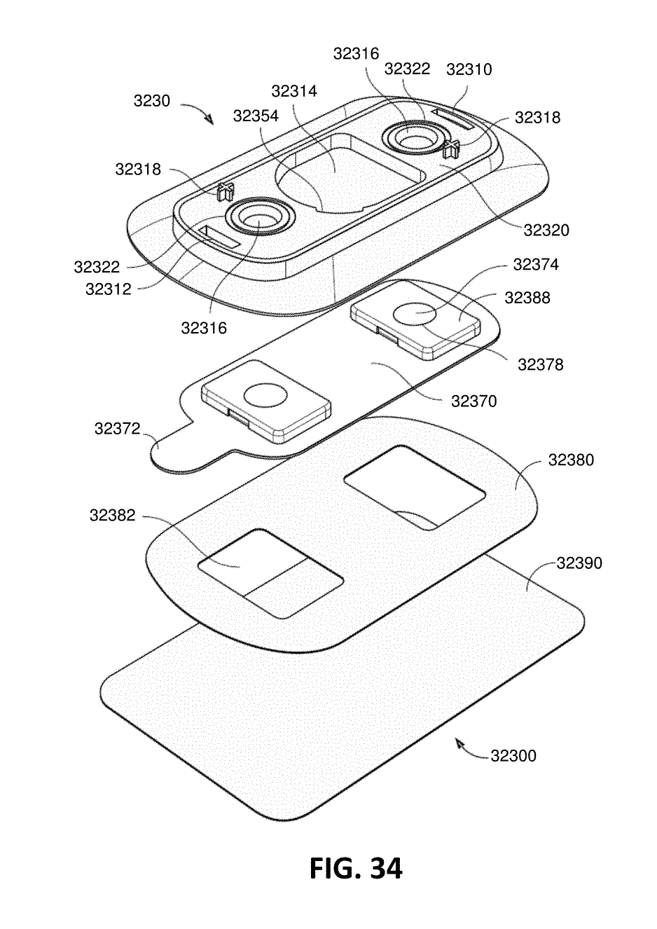

Another view of the reservoir module 3230 appears in FIG. 34. In this view, the electrodes 32374 are viewed through the apertures 32378 in the reservoir compartments 32388. Notable in FIG. 34 is the recess 32314 has an indent 32354, which is adapted to accept a complementary feature on the underside of an electrical module. This is one of many possible keying that may be provided for the device. In some embodiments, the recess 32314 may receive the underside of a battery compartment in the electrical module; however the person skilled in the art will recognize that many such keying features are possible. One such keying feature may be the dimensions of the snap receptacles 32310, 32312 and the corresponding snaps, which permit assembly of the two modules in one configuration only. Other keying features could include the size and/or position of the electrical inputs 32316 on the reservoir module 3230 and the corresponding electrical outputs on the electrical module, the size and/or positions of the power-on posts 32318, the complementary shapes of the reservoir module 3230 and the electrical module 3220.

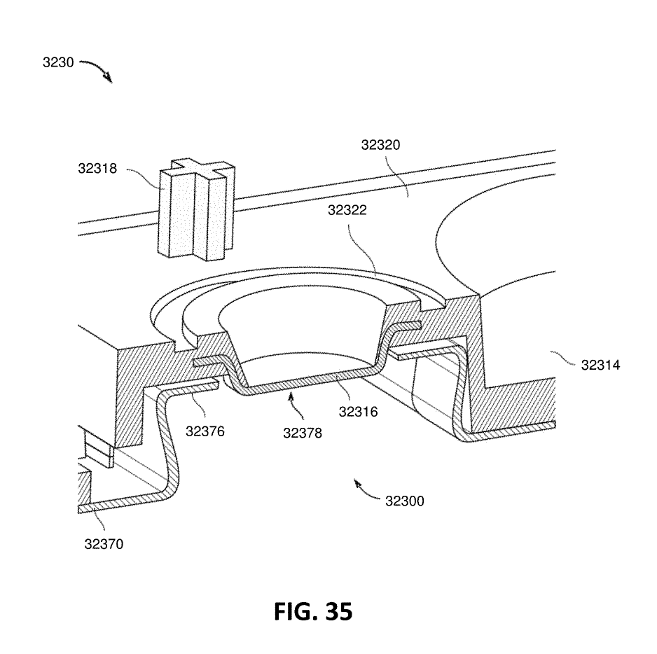

FIG. 35 is a cross section perspective view of an input connector 32316 on a reservoir module 3230. Visible in this view are the upper surface 32320 of the reservoir body 32300. Circumscribing the input connector 32316 is a seal 32322. The seal 32322 is configured to contact a corresponding seal on an electrical module to prevent ingress of contaminants upon assembly of the device. The contact 32316 is in some embodiments advantageously a planar (flat or substantially flat) metallic contact. The contact may be essentially any conductive metal, such as copper, brass, nickel, stainless steel, gold, silver or a combination thereof. In some embodiments, the contact is gold or gold plated.

Also visible on the upper surface 32320 of the reservoir module 3230 is a power-on post 32318 protruding from the surface 32320. The lower portion of input connector 32316 is configured to contact a reservoir (not pictured) through an aperture 32378 in the reservoir compartment 32388 in the electrode housing 32370.

Additionally, part of the battery receptacle 32314 may be seen in FIG. 35.

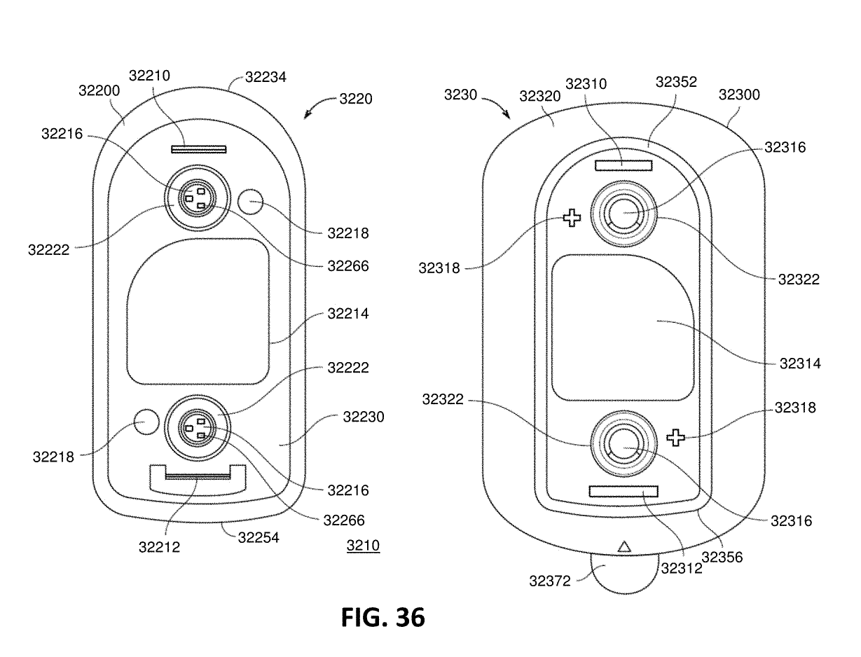

FIG. 36 is another view of the two modules 3220, 3230 side by side. On the left side of FIG. 36 is the bottom side of the electrical module body 32200; and on the right side is the top side of the reservoir module 3230. The bottom surface 32230 of electrical module body 32200 has snaps 32210, 32212 protruding therefrom, which are sized and shaped to fit within the snap receptacles 32310, 32312 on the top of the reservoir module body 32300. As discussed above, in some embodiments snaps 32210 and 32212 are of different size so that snap 32210 will not fit within snap receptacle 32312 and/or snap 32212 will not fit within snap receptacle 32310. This is one of several keying features that may be incorporated in the device 3210. As an illustrative example, snap 32212 cannot fit into 32310, because snap 32212 is larger than receptacle 32310; but snap 32210 can fit into receptacle 32312, because it is the smaller snap and larger receptacle. In other embodiments, it is possible to size both snaps and receptacles so that the one snap/receptacle pair is larger in one dimension (e.g., horizontally), while the other snap/receptacle pair is larger in the other dimension (e.g., longitudinally). Another keying feature is the protrusion 32214, which may house the battery or other component, and which is shaped to fit in one configuration within recess 32314 only.

The snaps 32210, 32212 are at least in some embodiments one-way snaps, meaning that they are biased so as to fit within the receptacles 32310, 32312 in such a way that they are not easily removed, and in at least some preferred embodiments, are configured to break (or deform to the extent that they are no longer operable) if forced apart so that the modules 3220, 3230 cannot be reassembled to form a single unitary device. In some embodiments, such a feature is provided as an anti-abuse character to the device, such that the reservoir module 3230 cannot be saved after use and employed with a different (or the same) electrical module 3220.

The lower surface 32230 of electrical module body 32200 also has two electrical outputs 32216, which are also referred to herein as output “hats”, which in certain embodiments are have one or more bumps 32266 protruding from the surface thereof. These hats 32216 are circumscribed by hat seals 32222. The hats 32216 are configured to make contact with the input connectors 32316 on the reservoir body 32300. Additionally, the hat seals 32222 are configured to contact and create an impermeable seal with the input seals 32322. Advantageously the hat seals 32222 are made of an elastomeric material that creates a contaminant-impermeable seal around the hats 32216 and, when mated with the input connector seals 32322, creates further contaminant-impermeable seals.

The power-on receptacles 32218 are configured to receive input posts 32318. In some embodiments, the power-on receptacles 32218 are made of a deformable (e.g. elastomeric) material. In some such embodiments, the power-on posts 32318 deform the power-on receptacles 32218 so that they contact power-on contacts (described in more detail below) and move them to a closed position, thereby connecting the battery into the circuit. Once the two modules 3220, 3230 are snapped together, the posts maintain pressure on the power-on contacts through the receptacles 32218 and keep the battery in the circuit.

While the hats 32216 and input contacts 32316 are depicted in FIG. 36 as being essentially the same size and symmetrically disposed along the longitudinal axis of the device 3210, another keying feature may be introduced into the device by changing the relative size and/or position with respect to the longitudinal axis of the hats 32216 and contacts 32316, the power-on posts 32318 and receptacles 32218, etc.

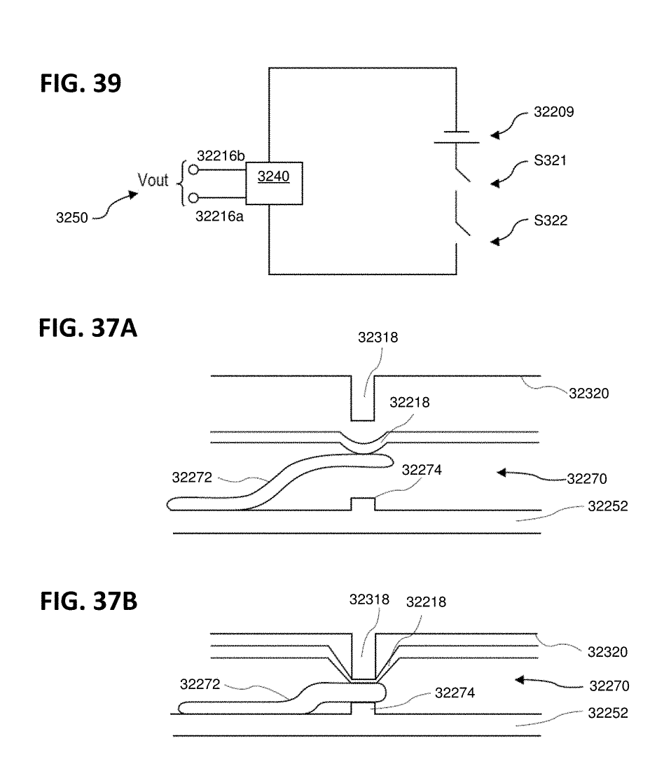

A cross section of one embodiment of a power-on switch 32270 is depicted in FIGS. 37A and 37B. The power-on switch 32270 comprises movable contact 32272 and a stationary contact 32274. Each of the movable contact 32272 and the stationary contact 32274 is connected to a portion of the circuitry on the printed circuit board (PCB) 32252. In the open position depicted in FIG. 37A, the movable contact 32272 is biased away from the stationary contact 32274, whereas in the closed position depicted in FIG. 37B, the two contacts 32272 and 32274 are pressed together by the power-on post 32318, which protrudes from the upper surface 32320 of the reservoir module 3230. The power-on post 32318 acts through the flexible (elastomeric) power-on receptacle 32218 to force the movable contact 32272 down until it is in contact with the stationary contact 32274. For the sake of visibility, the stationary contact 32274 is shown elevated from the PCB 32252; however, it will be understood that the stationary contact 32274 need not be, and generally will not be, elevated from the PCB 32252. In at least some embodiments, the stationary contact 32274 will be an exposed metal trace on the surface of the PCB 32252, though other configurations are also possible. The stationary contact 32272 is manufactured from a suitably springy metal, such as a copper alloy, which is biased to remain in the first, open position unless acted on by the power-on post 32318. The receptacle 32218 may resemble a dome when viewed from the side of facing the contacts 32272, 32274, and is at least in some embodiments formed of a suitable elastomeric substance that permits the power-on post 32318 to deform it without rupturing the seal. In some embodiments, the receptacle 32218 may also be planar or may be domed in the opposite direction. In at least some embodiments, the receptacle 32218 provides a contaminant-tight seal between the external and internal parts of the electrical module 3220.

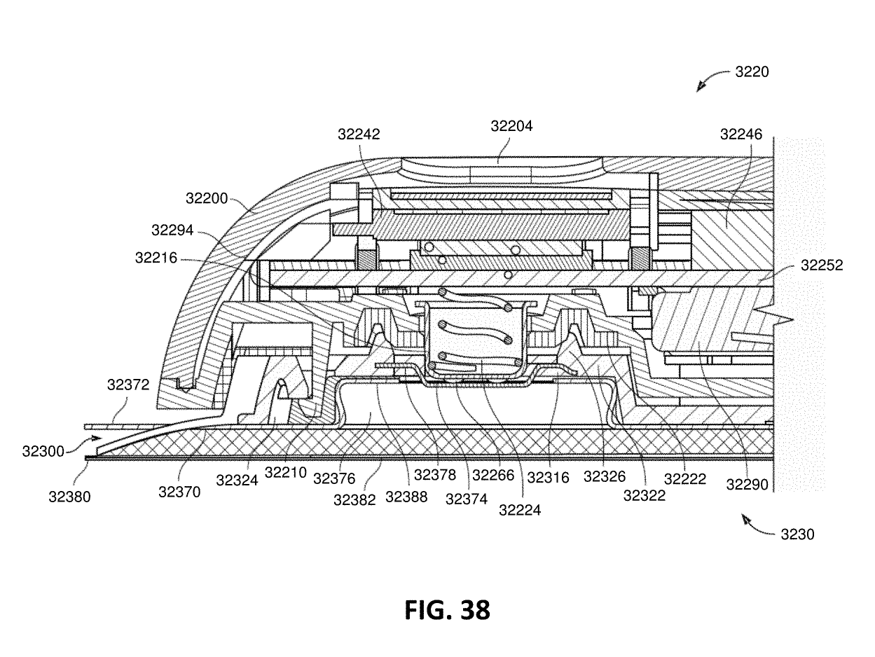

FIG. 38 shows a cross section of a part of a device 3210 in an assembled state. The device 3210 comprises the upper electrical module 3220, comprising an upper body 32200, and the reservoir module 3230, comprising reservoir body 32300, which are shown in this cross section view as combined. Parts of the electrical module 3220 that are visible in this cross section view include the electrical module body 32200, which contains a sound transducer 32246, an LCD 32204, controller 32242, and battery 32290, all of which are on the printed circuit board (PCB) 32252. A flex circuit 32294 provides a connection between the PCB 32252 and the LCD 32204. Also visible are the contact hat 32216, which has bumps 32266, and snap 32210. As can be seen, the contact hat 32216 is biased toward the reservoir module 3230 by a coil spring 32224, which fits within the contact hat 32216 and exerts a force through the contact hat 32216 to press the contact hat 32216 against the input connector 32316 of the reservoir module 3230. The hat 32216 is circumscribed by a hat seal 32222, which contacts the hat 32216 through its full length of travel. In at least some embodiments, this hat seal 32222 is an elastomeric seal that provides a contaminant-tight fit between the hat seal 32222 and the hat 32216, whereby the electrical module 3220 is sealed against contaminants such as particles and fluids (e.g. humidity) in the environment.

The reservoir module 3230 includes a reservoir 32376 and an electrode 32374 within the reservoir compartment 32388 in the electrode housing 32370, which also has an electrode housing tab 32372. In the assembled state, the snap 32210 catches on the ledge 32324 of the snap receptacle 32310. At least in some embodiments, the snap 32210 is made of a resilient polymer and is biased to maintain contact with the ledge 32324 so that the two modules 3220, 3230 cannot be easily separated. In some preferred embodiments, the snap 32210 is configured so that if the two modules 3220, 3230 are separated, the snap 32210 (and/or the ledge 32324) will break (or deform to the extent that they are no longer operable) and thereafter be unable to couple the two modules together.

Also depicted in this view is an input connector seal 32322, which in this illustration forms a ridge 32326 (input connector seal ridge) that circumscribes the input connector 32316. When the two modules 3220, 3230 are assembled, this input connector seal ridge 32326 contacts and presses into the elastomeric hat seal 32222, thereby preventing ingress of contaminants, such as particulates and liquids, into the space containing the output contact hat 32216 and the input contact 32316.

The hat 32216 projects through the aperture 32378 in the reservoir compartments 32388. At least the bumps 32266 on the hat 32216 contact the input connector 32316 to provide electrical contact between the electrical module 3220 and the reservoir module 3230. The spring 32224 provides mechanical bias to force the bumps 32266 to maintain contact with the input connector 32316. Although the hat 32216 is shown being biased by a coil spring 32224, the person having skill in the art will recognize that other springs and spring-like devices can be used within the scope of the device described herein. For example, and without limitation, the coil spring 32224 could be replaced by a beam spring or similar device.

As can be seen in FIG. 39, which is a high level schematic diagram of the electronics 3250 within the electrical module 3220, the electronics 3250 can be envisioned as including circuitry 3240 (which includes the controller, various indicators, etc.) connected to the battery 32290 through power-on switches S321 and S322 (which correspond to power-on switch 32270 in FIGS. 37A, 37B). The circuitry 3240 controls delivery of voltage Vout through the outputs 32216a, 32216b, which connect to corresponding inputs on the reservoir module. It is to be understood that, although the configuration of power-on switches S321 and S322 shown in FIGS. 37A and 37B is considered to provide certain advantages, such as ease of operation and manufacture, other configurations of switches may be employed within the scope of the device described herein. Such switches may include slides switches that are mechanically biased toward the open position, which may be pushed to the closed position by a power-on post or similar actuator. As can be seen in this figure, the circuit 3250 comprising the battery 32209 and the rest of the circuitry 3240 is only completed if both S321 and S322 are both held closed. Prior to S321 and S322 being closed, e.g. through the mechanical action of power-on posts, the battery 32290 is isolated from the circuitry 3240, as the circuit is open and does not allow current to flow through it. As mentioned before, this reduces battery drain prior to use and greatly reduces corrosion, as the circuitry has no power supply, and thus no extrinsic charge, applied to it. Also, if during handling prior to use one of the switches happens to close, e.g. for a brief period of time, the device will not power on. At least in some embodiments, it is considered advantageous for the controller to detect spurious short-lived closing of both switches S321 and S322 in order to account for occasional, accidental closing of the switches before use. Also, as discussed above, it is considered advantageous in some embodiments that the two switches S321 and S322 be physically and/or electrically remote from one another. Separation of the two switches reduces the likelihood that something that causes one of the switches to malfunction (e.g. close, whether permanently, reversibly or intermittently) will not also affect the other switch. Additionally or alternatively, the two switches may be located on two different sides of the battery or on the same side of the battery. Thus, while in FIG. 39 the switches S321, S322 are depicted on the positive (+) side of the battery 32290, one or both could be located on the other side of the battery. Thus, 1, 2, 3 or more switches may be located on one (positive or negative) side of the battery and 0, 1, 2, 3 or more switches may be located on the other (negative or positive) side of the battery. Physical separation of the two switches may be from 0.1 cm to several cm, and in some embodiments at least 0.5 cm.

Also apparent is FIG. 39 is that the switches S321, S322 are remote from the outputs 32216a, 32216b. Thus, the outputs from the electrical module to the reservoir module are separated from the switches S321, S322. Though in some preferred embodiments the closing of switches S321, S322 occurs as a result of the same action that connects the outputs 32216a, 32216b to the corresponding inputs on the reservoir module, the switches S321, S322 are remote from the outputs 32216a, 32216b. This allows switches S321, S322 to be entirely internal to the electrical module, and in some embodiments to be sealed against ingress of contaminants, such as water (including vapor) and/or particulates.

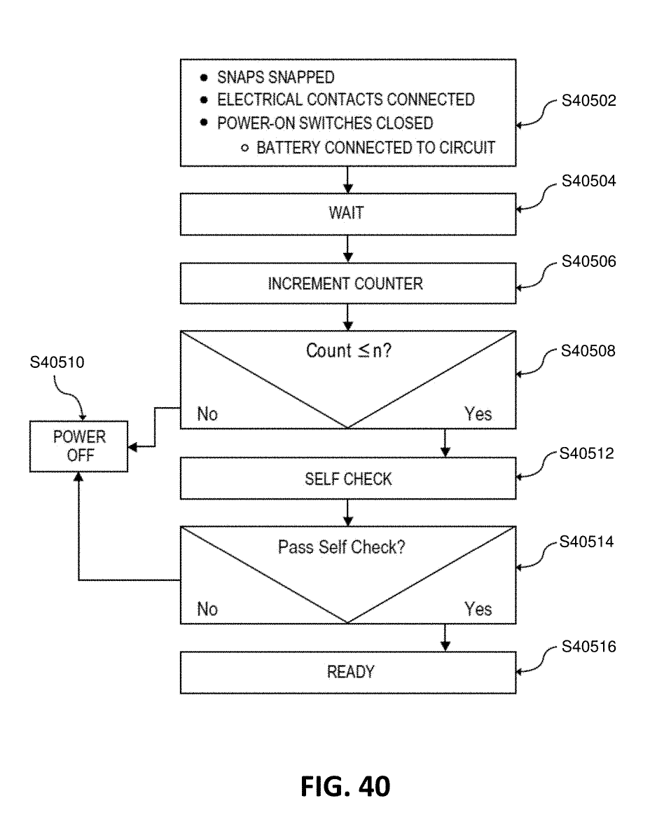

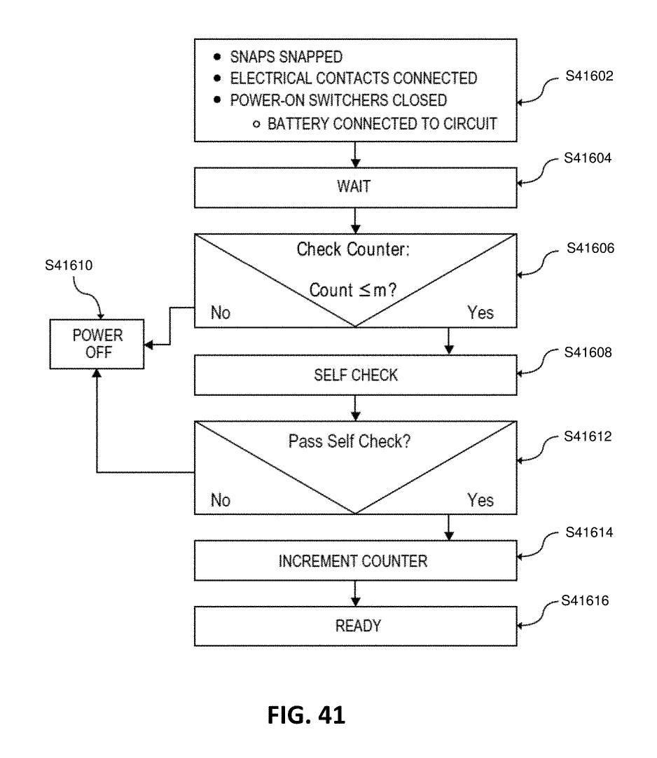

FIGS. 40 and 41 provide two alternative power-on sequences for a device 4010 according as described herein. The first alternative shows that in the first step, S40502, four events occur all at once in a single action by the user: the snaps are snapped into their respective receptacles; the output and input contacts are mated to provide electrical contact between the reservoirs in the reservoir module and the circuitry in the electrical module; the power-on posts close the power-on switches in the electrical module; and the battery is thereby connected into the circuit and begins providing power to the circuitry. In step S40504 the controller waits a minimum period of time (e.g. 10-500 ms) before proceeding to the next step. In some embodiments, S40504 is eliminated from the power-on sequence. In embodiments in which S40504 is included in the power-on sequence, if the controller fails to maintain power for a predetermined minimum period of time, that is, e.g. power is lost during this timeframe, the timer resets to zero. Presuming that power is maintained through the time period of step S40504, the controller then increments the power-on counter by 1 in step S40506. In step S40508, the controller then checks the number of counts on the power-on counter, and if it is less than or equal to a certain predetermined number (in this example 2, presuming that the counter had been set to 1 by an in-factory test, though other values are possible) the controller proceeds to step S40510, which includes a self-check. If, however, the count is greater than the predetermined number, then the controller initiates step S40516, which includes a power off sequence, which may include sending an error message to an LCD display, activating an LED indicator and/or sounding an audible alarm. If the count is less than or equal to the predetermined number, the controller initiates step S40510. After the self-check of S40510 is completed, the controller determines whether the circuitry has passed the self-check, and if not, it initiates step S40516. If the circuitry passes the self-test check, the controller then initiates S40512, which may include signaling the user that the device is ready (e.g. through the LCD, LED and/or sound transducer). The device is then ready to be applied to the body of a patient and operated normally, e.g. as described in U.S. Pat. No. 6,216,033 B1, which is incorporated herein by reference in its entirety.

A second alternative in FIG. 41 shows that in the first step, S41602, four events occur all at once in a single action by the user: the snaps are snapped into their respective receptacles; the output and input contacts are mated to provide electrical contact between the reservoirs in the reservoir module and the circuitry in the electrical module; the power-on posts close the power-on switches in the electrical module; and the battery is thereby connected into the circuit and begins providing potential to the circuitry. In step S604 the controller waits a minimum period of time (e.g. 10-500 ms) before proceeding to the next step. If the controller fails to maintain power for this period of time, that is, power is lost during this timeframe, the timer resets to zero. Presuming that power is maintained through the time period of step S41604, the controller then checks the number of counts on the power-on counter in S41606, and if it is less than or equal to a certain predetermined number (in this example 1, presuming that the counter had been set to 1 by an in-factory test, though other values are possible) the controller proceeds to step S41610, which includes a self-check. If, however, the count is greater than the predetermined number, then the controller initiates step S41616, which includes a power off sequence, which may include sending an error message to an LCD display, activating an LED indicator and/or sounding an audible alarm. If the count is less than or equal to the predetermined number, the controller initiates step S41610. After the self-check of S41610 is completed, the controller determines whether the circuitry has passed the self-check, and if not, it initiates step S41616. If the circuitry passes the self-test check, the controller then initiates S41612, which includes incrementing the counter by 1. The controller then initiates S41614, which may include signaling the user that the device is ready (e.g. through the LCD, LED and/or sound transducer).

The device is then ready to be applied to the body of a patient and operated normally, e.g. as described in U.S. Pat. No. 6,216,033 B1, which is incorporated herein by reference in its entirety.

Briefly described, the device is applied to the surface of a patient's skin. The patient or a healthcare professional may then press the button 32202 (see, e.g., FIGS. 32A, 32B, and 33). In some embodiments, the device is configured to require the patient or healthcare professional to press the button twice within a predetermined timeframe in order to prevent accidental or spurious administration of the therapeutic agent. Provided the patient or healthcare professional properly presses the button 32202, the device 3210 then begins administering the therapeutic agent to the patient. In between the doses, the device may enter a “Ready” mode during which the delivery is “off” even though the device is powered on. While in the Ready mode, the device may also perform a number of self-test including the off-current self-test described above. If the user presses the button to receive another dose, the device may first perform one or more self-tests (including the off-current self-test) before delivering the dose (entering the activated state and delivering dosage by passing current between the anode and cathode). Once a predetermined number of doses have been administered and/or a predetermined period of time has elapsed since the device was powered on, the device initiates a power off sequence, which may include sending a power off signal to the user through an LCD display, an LED and/or an audio transducer. See especially the claims of U.S. Pat. No. 6,216,033 B1, which are incorporated herein by reference.

The person skilled in the art will recognize that other alternative power-on sequences may be employed. For example, the controller may increment the counter immediately after the counter check in the process outlined in FIG. 40 or 41.

The reservoir of the electrotransport delivery devices generally contain a gel matrix, with the drug solution uniformly dispersed in at least one of the reservoirs. Other types of reservoirs such as membrane confined reservoirs are possible and contemplated. The application of the present invention is not limited by the type of reservoir used. Gel reservoirs are described, e.g., in U.S. Pat. Nos. 6,039,977 and 6,181,963, which are incorporated by reference herein in their entireties. Suitable polymers for the gel matrix can comprise essentially any synthetic and/or naturally occurring polymeric materials suitable for making gels. A polar nature is preferred when the active agent is polar and/or capable of ionization, so as to enhance agent solubility. Optionally, the gel matrix can be water swellable nonionic material.

Examples of suitable synthetic polymers include, but are not limited to, poly(acrylamide), poly(2-hydroxyethyl acrylate), poly(2-hydroxypropyl acrylate), poly(N-vinyl-2-pyrrolidone), poly(n-methylol acrylamide), poly(diacetone acrylamide), poly(2-hydroxylethyl methacrylate), poly(vinyl alcohol) and poly(allyl alcohol). Hydroxyl functional condensation polymers (i.e., polyesters, polycarbonates, polyurethanes) are also examples of suitable polar synthetic polymers. Polar naturally occurring polymers (or derivatives thereof) suitable for use as the gel matrix are exemplified by cellulose ethers, methyl cellulose ethers, cellulose and hydroxylated cellulose, methyl cellulose and hydroxylated methyl cellulose, gums such as guar, locust, karaya, xanthan, gelatin, and derivatives thereof. Ionic polymers can also be used for the matrix provided that the available counterions are either drug ions or other ions that are oppositely charged relative to the active agent.

Incorporation of the drug solution into the gel matrix in a reservoir can be done in any number of ways, i.e., by imbibing the solution into the reservoir matrix, by admixing the drug solution with the matrix material prior to hydrogel formation, or the like. In additional embodiments, the drug reservoir may optionally contain additional components, such as additives, permeation enhancers, stabilizers, dyes, diluents, plasticizer, tackifying agent, pigments, carriers, inert fillers, antioxidants, excipients, gelling agents, anti-irritants, vasoconstrictors and other materials as are generally known to the transdermal art. Such materials can be included by on skilled in the art.

The drug reservoir can be formed of any material as known in the prior art suitable for making drug reservoirs. The reservoir formulation for transdermally delivering cationic drugs by electrotransport is preferably composed of an aqueous solution of a water-soluble salt, such as HCl or citrate salts of a cationic drug, such as fentanyl or sufentanil. More preferably, the aqueous solution is contained within a hydrophilic polymer matrix such as a hydrogel matrix. The drug salt is preferably present in an amount sufficient to deliver an effective dose by electrotransport over a delivery period of up to about 20 minutes, to achieve a systemic effect. The drug salt typically includes about 0.05 to 20 wt % of the donor reservoir formulation (including the weight of the polymeric matrix) on a fully hydrated basis, and more preferably about 0.1 to 10 wt % of the donor reservoir formulation on a fully hydrated basis. In one embodiment the drug reservoir formulation includes at least 30 wt % water during transdermal delivery of the drug. Delivery of fentanyl and sufentanil has been described in U.S. Pat. No. 6,171,294, which is incorporated by reference herein. The parameter such as concentration, rate, current, etc. as described in U.S. Pat. No. 6,171,294 can be similarly employed here, since the electronics and reservoirs of the present invention can be made to be substantially similar to those in U.S. Pat. No. 6,171,294.

The drug reservoir containing hydrogel can suitably be made of any number of materials but preferably is composed of a hydrophilic polymeric material, preferably one that is polar in nature so as to enhance the drug stability. Suitable polar polymers for the hydrogel matrix include a variety of synthetic and naturally occurring polymeric materials. A preferred hydrogel formulation contains a suitable hydrophilic polymer, a buffer, a humectant, a thickener, water and a water soluble drug salt (e.g. HCl salt of a cationic drug). A preferred hydrophilic polymer matrix is polyvinyl alcohol such as a washed and fully hydrolyzed polyvinyl alcohol (PVOH), e.g. MOWIOL 66-100 commercially available from Hoechst Aktiengesellschaft. A suitable buffer is an ion exchange resin which is a copolymer of methacrylic acid and divinylbenzene in both an acid and salt form. One example of such a buffer is a mixture of POLACRILIN (the copolymer of methacrylic acid and divinyl benzene available from Rohm & Haas, Philadelphia, Pa.) and the potassium salt thereof. A mixture of the acid and potassium salt forms of POLACRLIN functions as a polymeric buffer to adjust the pH of the hydrogel to about pH 6. Use of a humectant in the hydrogel formulation is beneficial to inhibit the loss of moisture from the hydrogel. An example of a suitable humectant is guar gum. Thickeners are also beneficial in a hydrogel formulation. For example, a polyvinyl alcohol thickener such as hydroxypropyl methylcellulose (e.g. METHOCEL K100 MP available from Dow Chemical, Midland, Mich.) aids in modifying the rheology of a hot polymer solution as it is dispensed into a mold or cavity. The hydroxypropyl methylcellulose increases in viscosity on cooling and significantly reduces the propensity of a cooled polymer solution to overfill the mold or cavity.

Polyvinyl alcohol hydrogels can be prepared, for example, as described in U.S. Pat. No. 6,039,977. The weight percentage of the polyvinyl alcohol used to prepare gel matrices for the reservoirs of the electrotransport delivery devices, in certain embodiments can be about 10% to about 30%, preferably about 15% to about 25%, and more preferably about 19%. Preferably, for ease of processing and application, the gel matrix has a viscosity of from about 1,000 to about 200,000 poise, preferably from about 5,000 to about 50,000 poise. In certain preferred embodiments, the drug-containing hydrogel formulation includes about 10 to 15 wt % polyvinyl alcohol, 0.1 to 0.4 wt % resin buffer, and about 1 to 30 wt %, preferably 1 to 2 wt % drug. The remainder is water and ingredients such as humectants, thickeners, etc. The polyvinyl alcohol (PVOH)-based hydrogel formulation is prepared by mixing all materials, including the drug, in a single vessel at elevated temperatures of about 90 degree C. to 95 degree C. for at least about 0.5 hour. The hot mix is then poured into foam molds and stored at freezing temperature of about −35 degree C. overnight to cross-link the PVOH. Upon warming to ambient temperature, a tough elastomeric gel is obtained suitable for ionic drug electrotransport.

A variety of drugs can be delivered by electrotransport devices. In certain embodiments, the drug is a narcotic analgesic agent and is preferably selected from the group consisting of fentanyl and related molecules such as remifentanil, sufentanil, alfentanil, lofentanil, carfentanil, trefentanil as well as simple fentanyl derivatives such as alpha-methyl fentanyl, 3-methyl fentanyl and 4-methyl fentanyl, and other compounds presenting narcotic analgesic activity such as alphaprodine, anileridine, benzylmorphine, beta-promedol, bezitramide, buprenorphine, butorphanol, clonitazene, codeine, desomorphine, dextromoramide, dezocine, diampromide, dihydrocodeine, dihydrocodeinone enol acetate, dihydromorphine, dimenoxadol, dimeheptanol, dimethylthiambutene, dioxaphetyl butyrate, dipipanone, eptazocine, ethylmethylthiambutene, ethylmorphine, etonitazene, etorphine, hydrocodone, hydromorphone, hydroxypethidine, isomethadone, ketobemidone, levorphanol, meperidine, meptazinol, metazocine, methadone, methadyl acetate, metopon, morphine, heroin, myrophine, nalbuphine, nicomorphine, norlevorphanol, normorphine, norpipanone, oxycodone, oxymorphone, pentazocine, phenadoxone, phenazocine, phenoperidine, piminodine, piritramide, proheptazine, promedol, properidine, propiram, propoxyphene, and tilidine.

Some ionic drugs are polypeptides, proteins, hormones, or derivatives, analogs, mimics thereof. For example, insulin or mimics are ionic drugs that can be driven by electrical force in electrotransport.

For more effective delivery by electrotransport salts of certain pharmaceutical analgesic agents are preferably included in the drug reservoir. Suitable salts of cationic drugs, such as narcotic analgesic agents, include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, .beta.-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate. The more preferred salt is chloride.

A counterion is present in the drug reservoir in amounts necessary to neutralize the positive charge present on the cationic drug, e.g. narcotic analgesic agent, at the pH of the formulation. Excess of counterion (as the free acid or as a salt) can be added to the reservoir in order to control pH and to provide adequate buffering capacity. In one embodiment of the invention, the drug reservoir includes at least one buffer for controlling the pH in the drug reservoir. Suitable buffering systems are known in the art.

The device described herein is also applicable where the drug is an anionic drug. In this case, the drug is held in the cathodic reservoir (the negative pole) and the anoidic reservoir would hold the counterion. A number of drugs are anionic, such as cromolyn (antiasthmatic), indomethacin (anti-inflammatory), ketoprofen (anti-inflammatory) and ketorolac tromethamine (NSAID and analgesic activity), and certain biologics such as certain protein or polypeptides.

Although the device and systems for drug delivery including an off-current self-test (and therefore an off-current module to perform the self-test) may be or include two-part drug delivery devices as descried above, the off-current module may be included as part of virtually any drug delivery system having a powered on, but delivery-off (e.g., “Ready”) mode in which drug is not to be delivered until appropriately triggered. Thus one-part, unitary drug delivery devices are also contemplated.

Any of the systems and devices describe herein, including a two-part system as exemplified may include logic for controlling the self-tests, including the off-current (aka anode-cathode voltage difference) self-test. Described in Example 2 below, and accompanying figures, is one variation of a system and control logic to be implemented on the system, including an off-current self-test. This exemplary logic includes an off-current module, and may be implemented on the two-part system described in Example 1, above.

### Example 2: Control Logic

In one example, a system/device including an off-current control module configured to include an off-current self-test may include a processor or other controller executing control logic. For convenience, this control logic is referred to herein as software, however it should be understood that it may include hardware, firmware, or the like, in addition to software.

The following acronyms used in this example are defined below:

| Term | Definition |
|---|---|
| ITSIC | ASIC designed and produced for/by this example |
| ASIC | Application-Specific Integrated Circuit |
| IONSYS ™ | Fentanyl Iontophoretic Transdermal System |
| ITSIC | Specific Integrated Circuit (formerly called ALZIC) for |
| | this example |
| JTAG | (Joint Test Action Group) An interface to the ITSIC that |
| | allows access and control by external equipment |
| Nibble | Half of an 8-bit byte. Four bits aligned on bit zero or bit |
| | four of an 8-bit byte |
| Syndrome Bit | Hamming Code parity bit |
| TDI | Technical Design Input |
| UML | Unified Modeling Language |

In this example, the software (control logic) described herein may be run on the ITSIC ASIC, which contains a CAST R80515 CPU core. In addition to the core, the ITSIC contains peripherals for interfacing with input/output devices including buttons, LEDs, an LCD, and a piezo transducer. The ITSIC also includes a high-voltage boost converter, a current source, and an analog-to-digital converter (ADC).

The exemplary CAST R80515 core operates at 32 kHz and takes between one and six cycles to execute each instruction. This equates to execution times ranging from 31.25 to 187.5 μs per instruction. The ITSIC contains 256 bytes of RAM, of which 32 bytes are reserved for core registers, 1024 bytes of non-volatile storage in the form of EEPROM arranged in 64-bit pages, and 16 KB of ROM for program memory. The ITSIC can execute code from program memory in internal ROM, or from external EEPROM. The transfer of execution from internal ROM to external EEPROM is controlled by a hardware register setting that may be configured via JTAG or by software.

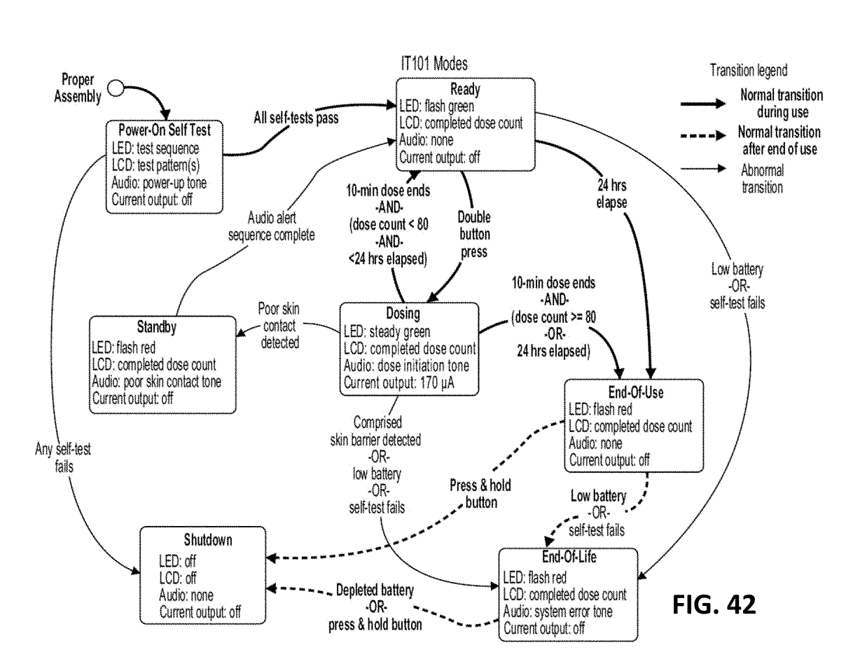

The IT101 may operate in one of seven modes, determined by user input, defined operational parameters, and device internal status. FIG. 42 shows the behavior of each mode and the transitions between modes.

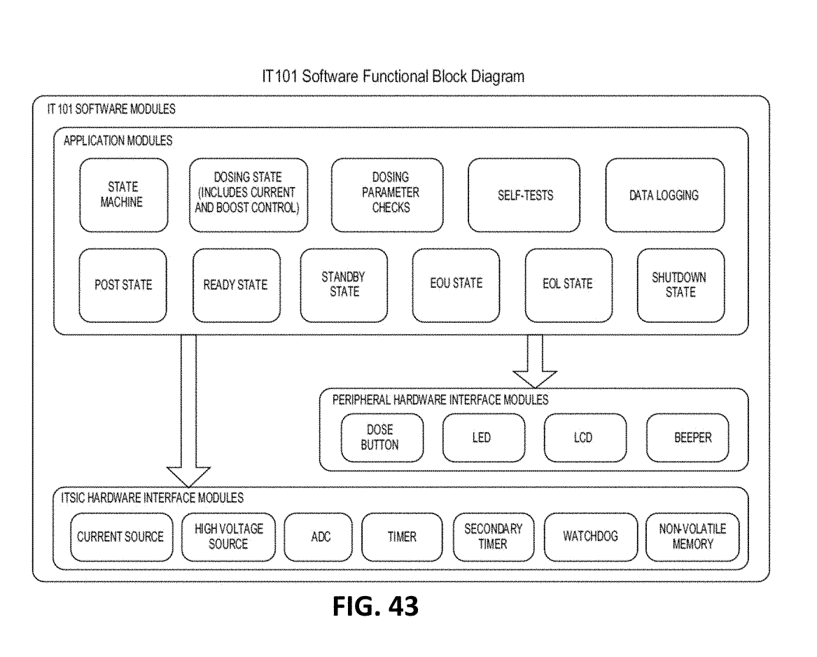

FIG. 43 shows the high-level decomposition of the software into functional blocks. The software architecture in this example is modular and layered, with low-level driver modules encapsulating and providing an interface to electronic hardware, while higher-level application modules utilize drivers to provide device functionality to the user. Lower-layer modules are independent of modules in layers above them.

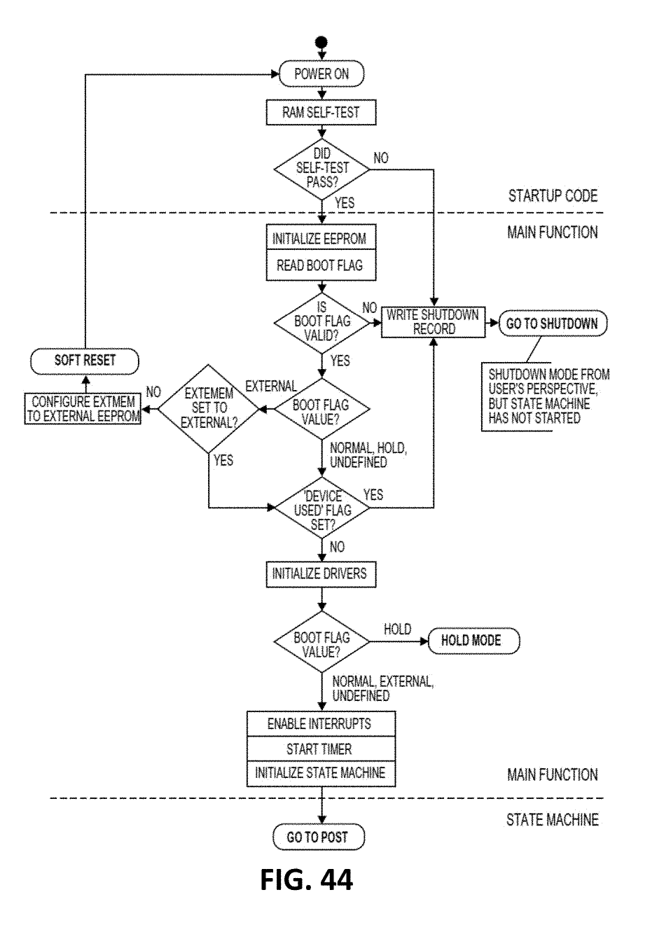

Before entering the state machine, the software goes through an initialization routine. This routine includes checking the RAM and EEPROM for corruption, checking the boot mode, and initializing the drivers. More details of this initialization can be seen in FIG. 44.

The ITSIC supports execution from either internal Mask ROM or an external EEPROM. The default configuration is execution from ROM. In addition, the software includes a Hold Mode which initializes the system then enters an infinite loop to allow external control via the JTAG lines. Hold mode does not service the watchdog timer, so if external control isn't asserted prior to the first expiration of the watchdog, the watchdog will reset the system. The boot mode of the system is determined by the boot flag in NVM.

During system initialization, the EEPROM is initialized and the first page is checked for data integrity. If the boot flag value is not corrupt, the value is read from EEPROM.

If the flag is set to Normal, the software continues to run from ROM. If the flag is set to External, the EXTMEM register is set by software, which resets the CPU and subsequently boots from external EEPROM. If the flag is set to Hold, the drivers are first initialized, and then the software enters Hold Mode.

Processing of tasks in the system may be periodic and synchronized with a system tick occurring every eight milliseconds. The system tick function is provided by the Timer driver, using a periodic hardware interrupt to produce the tick. The main loop simply waits for the system tick to occur, then calls the appropriate processing functions for the Timer driver and the state machine.

The Timer processing function updates any active timers, such as those for dose time and system lifetime. The state machine processing function dispatches processing to the currently-active state, which then executes its periodic tasks. Periodic tasks may be scheduled to run as frequently as every 8 ms, or with any period that is an integer multiple of 8 ms, up to 2.048 seconds. The upper limit on the period is fixed by rollover of the 8-bit system tick counter. The Timer driver provides functions to facilitate periodic execution at various rates. To reduce demands on the processor core, tasks may be scheduled to run at rates no faster than necessary.

There is a single thread of execution that executes tasks in a non-preemptive, run-to-completion model. The active task must complete before the next task can run, so no task is allowed to wait for an extended period for an event to occur. If execution of a particular task runs past the scheduled time for one or more other tasks, the delayed task(s) will be executed in order, upon completion of the delaying task. Execution of all periodic processing tasks will generally take longer than the duration of a single system tick. Normal scheduling will continue on the next system tick.

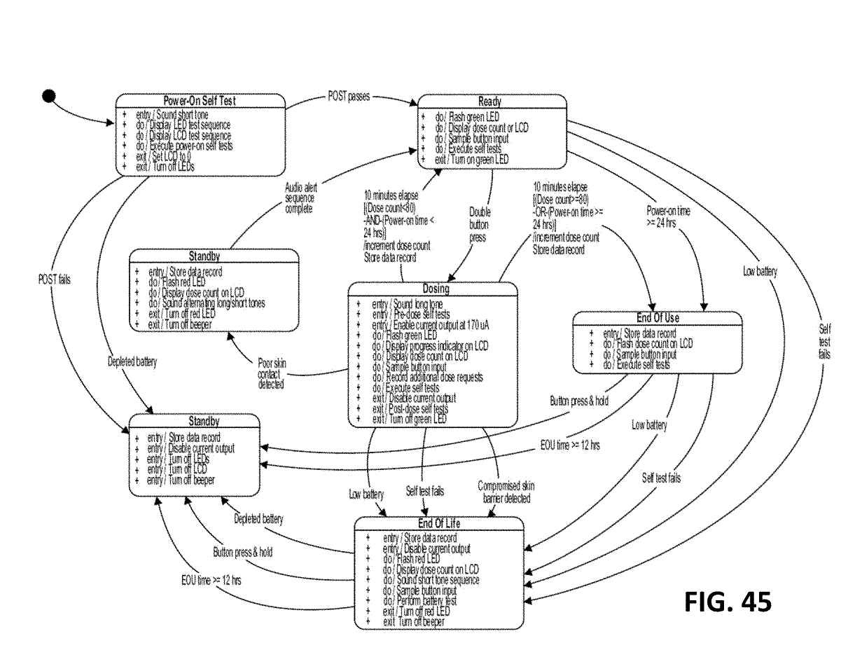

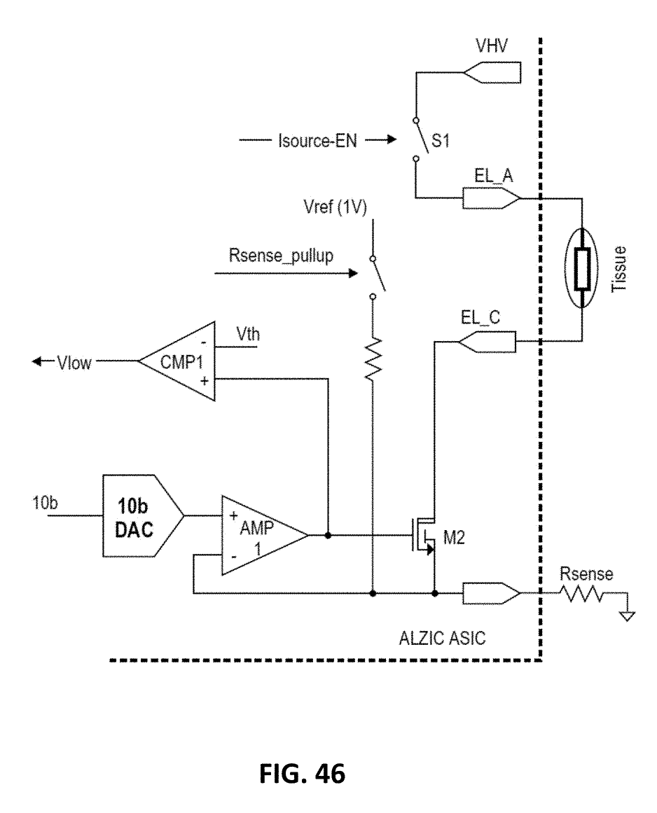

The software in this example operates as a finite state machine, the behavior of which is defined in the UML state chart shown in FIG. 45. The state machine is implemented with state processing and transitions managed centrally by the StateMachine module. Each state has entry and exit functions, as well as a processing function. The current state of the system is stored in a single private variable within the StateMachine module.

Each time a system tick occurs, the main loop calls the state machine processing function, which in turn calls the processing function for the current state. If processing of the current state results in a transition, the processing function returns a reference to the new state. The state machine then calls the exit function for the current state, changes the state variable, then calls the entry function for the new state. This assures that the state of the system remains consistent at all times, with guaranteed state entry and exit actions performed in the correct order. If a state's processing function does not result in a transition, it returns null, and no state change takes place.

Each state contains its own list of periodic tasks that are executed at the appropriate rates by its processing function. Tasks are scheduled in a rate-monotonic fashion—the periodic tasks with the highest rate of execution are executed first, followed by tasks in order of decreasing execution rate. This minimizes the variability in the period, particularly for the tasks with the highest execution rates. Task scheduling is static and fixed at compile time, so priority is deterministic.

### States

### Power-on Self-Test State

In the Power-On Self-Test (POST) state, the software exercises the user interface elements and executes a sequence of self-tests. At power-on, the beeper sounds a 250 ms, 2000 Hz tone. After the tone, the red LED flashes once for 500 ms. After the LED flash, the LCD flashes ‘88’ once per second for the remainder of POST.

While the user interface elements are being exercised, the software executes a sequence of self-tests to confirm that the device hardware is operating correctly. In order to complete POST as quickly as possible, the tests run continuously until they complete, rather than utilizing a periodic task for execution. There are two periodic tasks in the POST state. A 250-ms task is used to produce the user interface sequences. A one-second task is used to service the watchdog.

### Ready State

In the Ready state, the software looks for button input, flashes the green LED for a half second every two seconds and periodically runs self-tests according to schedule. There are three periodic tasks in the Ready state, executing with periods of 50 ms, 250 ms, and one second.

The 50-ms task is used to detect button presses, using the functions provided by the Button driver. The software looks for a dose request, defined as two button presses separated by at least 0.3 seconds and at most three seconds. The time is measured from the point of the first press to the point of the second release. On each detected button release, the software performs an Analog Switch Validation Test. When a dose request is detected, the software performs a Digital Switch Validation Test. If all tests pass, a transition to Dosing state is initiated.

The 250-ms task is used to produce the flashing sequence of the green LED. The green LED is turned on for half a second every two seconds.

The one-second task is used to schedule and execute self-tests, and service the watchdog.

### Dosing State

The Dosing State is responsible for delivering the 170 μA drug delivery current over the 10 minute dose. For reference, 16 illustrates one variation of a the circuit controlling the anode and cathode. The current control block contains circuitry to connect the output of the voltage boost converter (VHV) to the anode electrode (EL_A) through the switch S1. The 10 bit DAC is used to configure the current output to a set value proportional to the desired dosing current. The DAC drives AMP1 which controls the current flowing through EL_A and EL_C by driving the gate of M2. The drain of M2 determines the current flow through Rsense which causes the voltage drop that is fed back into AMP1. As the skin resistance between EL_A and EL_C varies, so does the current through Rsense, which triggers a change in the output of AMP1. The VLOW signal is used in mode 0 to monitor the output of AMP1 as it approaches the saturation point of 2 volts. AMP1 becomes saturated if there is not sufficient voltage to deliver the programmed current with the resistance between EL_A and EL_C. Driver functions are available to control and monitor various the points of this circuit.

The Dosing State is grouped into three sub-flows: dose initiation sequence, dose control and dose completion sequence. Upon transition from Ready state to Dosing state the dose initiation sub-flow is started. In dose initiation the software configures the various points of the current control block and verifies their proper operation. The dose control sub-flow is then started. This flow controls the device over the 10 minute dose, monitoring for error conditions and controlling boost voltage to conserve power. Finally, the dose completion sub-flow is started. This flow disables drug delivery and verifies correct operation of the current source by measuring the various points in the current control block.

The dose termination sequence is always run on exit of the Dosing state independent of the event that caused the software to exit the Dosing state. The dose termination sequence always opens 51, sets the current source DAC to 0, sets the boost voltage to 0 and disables the boost circuit. Further, the dose termination sequence disables both the green LED and beeper. In some cases the dose termination sequence carries out actions already completed in the sub-flow processing. Almost all error cases in the Dosing State flow are handled similarly—with a resulting transition to dose termination. The exception to this is the handling of Poor Skin Contact detection.

If an error occurs during dose initiation or dose completion the software exits the Dosing State, completes the dose termination sequence and transitions to End of Life. Likewise, if an error other than Poor Skin Contact is encountered during dose control, the software completes the dose termination sequence and transitions to End of Life. When a Poor Skin Contact error is encountered in the Dosing State, the software immediately starts the dose completion sequence, but the dose count is not updated. When an error occurs in the dose completion sequence the software immediately completes the dose termination sequence and transitions to End of Life.

There are three periodic tasks in the Dosing state, executing with periods of 50 ms, 500 ms and one second.

The 50-ms task is used to detect dose requests while in the dosing state. The double button press detection mechanism is identical to Ready mode, except switch validation tests are not run. If the software detects a double button press in the dosing state, the dose request counter is incremented. This count is logged during dose-completion, but not when handling a Poor Skin Contact error.

The 500-ms task is used only the first time its tick occurs. On that first occurrence, the beeper is disabled.

The one-second task in this example is used to schedule the dose control sub-flow and service the watchdog. The one second task also schedules the slower rate Dosing State self-tests (i.e. the ADC and Reference Voltage test, Oscillator Accuracy Test, Battery Voltage Test and Software Timer Integrity Test).

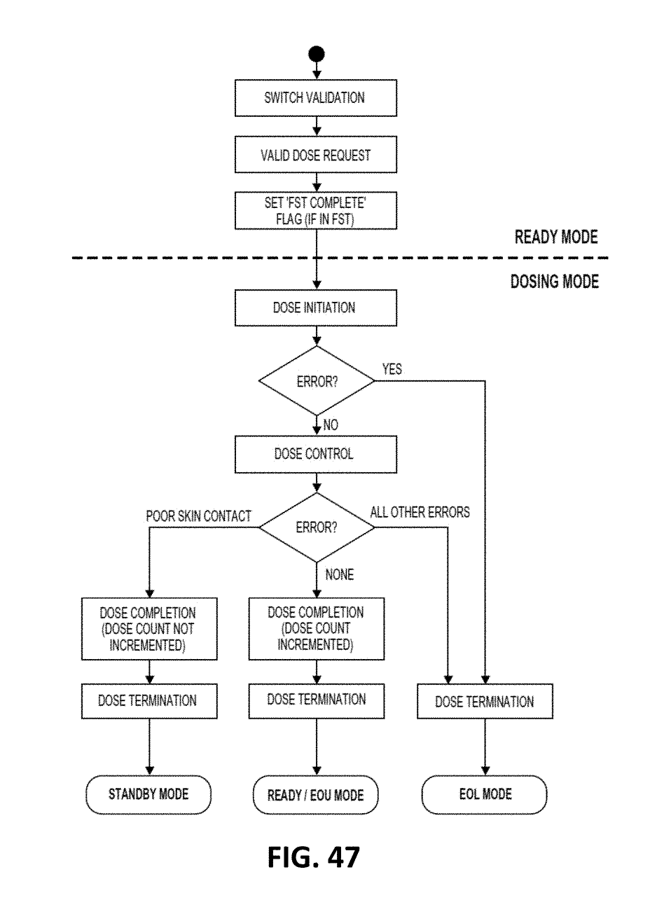

FIG. 47 shows a Dosing Mode Flow Diagram illustrating the high-level flow between each of the dosing mode sub-flows, the dose termination sequence and the transition to other states.

### Dose Initiation Sequence

The dose initiation sequence starts by completing the sequence of turning on the green LED and enables the piezo beeper at 2000 Hz for a duration of 500 milliseconds. The software then completes the required self-tests for dosing mode entry. At this point the software begins to configure the device for drug delivery.

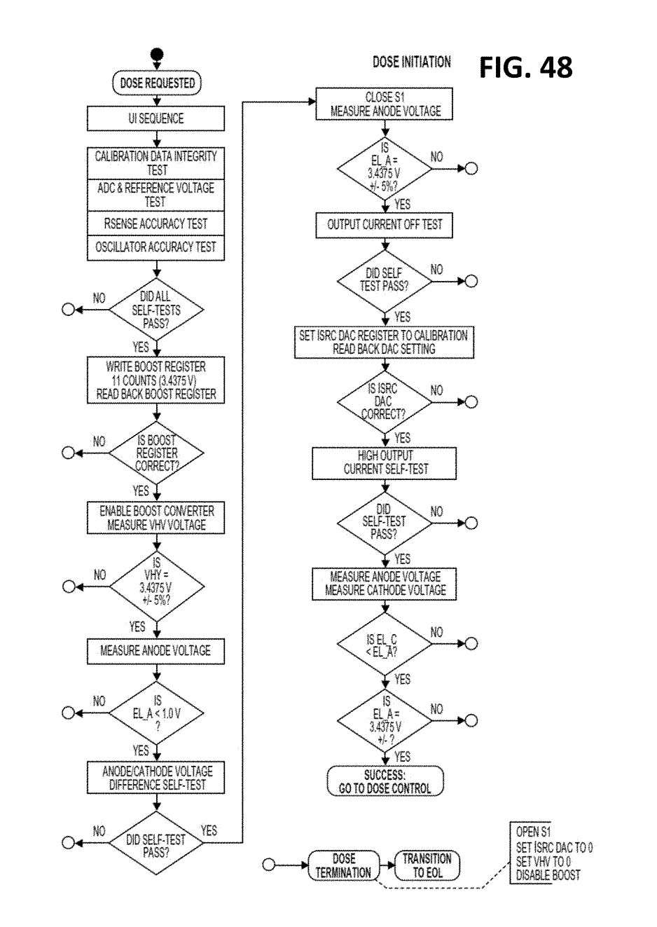

First the software writes the initial boot voltage setting of 3.4375 V and reads back the register to verify the write. Next, voltage boost is enabled and the software confirms that the boost circuit is operational by measuring the boost voltage using the ADC. The software then verifies that S1 is open by measuring the voltage on EL_A and confirming that it is below 1.0 V. The software verifies that there is not a large potential difference between the anode and cathode by completing the Anode/Cathode voltage difference test. Next, S1 is closed and the voltage on EL_A is measured again to confirm that S1 is closed. The software verifies that the output current is off by conducting the Output Current Off self-test. At this point the software sets the current source DAC to the calibrated value to start current flow. The software reads back the register to verify the write. The software next conducts the High Output Current self-test to verify that the current source is within range. Finally, the software measures both the anode and the cathode and conducts two checks. The first verifies that there is a voltage difference between EL_A and EL_C; the second verifies that the boost circuit is still able to supply the voltage with current enabled. If the measured values are not as expected, the software has detected an error, completes the dose termination sequence and transitions to End of Life. FIG. 48 shows a Dose Initiation Flow Diagram.

### Dose Control Sequence

Upon successful completion of the dose initiation sequence the software enters dose control. The software starts the dose countdown timer with a duration of 10 minutes and begins the dose control loop on a 1 second period.

Each time through the loop the software first verifies that the output current is below 187 μA by completing the High Output Current self-test. Next, the software verifies that EL_A is within tolerance of the current VHV setting. After 1 minute has elapsed the Compromised Skin Barrier Test is performed each time through the loop and after 4 minutes has elapsed the Poor Skin Contact Test is performed each time through the loop.

After the self-tests are completed the software enters the VHV control portion of the loop. The software controls VHV to provide enough voltage to deliver the drug current while minimizing power consumption. The VHV control loop ramps the voltage to the necessary level, starting at 3.4375 V but never going above 11.25 V. To control VHV the software monitors the state of the VLOW signal. The VLOW signal is configured to monitor the gate voltage of M2. The signal is asserted when the output of AMP1 exceeds 2 V. The VLOW signal indicates that AMP1 is not able to deliver the 170 μA current because there is not sufficient source voltage. If the VLOW signal is asserted, the software increments VHV by 1 count (0.3125 V), up to a maximum of 11.25 V. The first several iterations through the control loop ramp VHV to the necessarily level, depending on the skin resistance. If skin resistance increases during the dose, the VLOW signal is asserted and VHV is incremented accordingly.

To conserve power and handle decreasing skin resistance during dose delivery, the software decrements VHV periodically. The decrement is triggered by a 20 second timeout. The timeout is set to 0 each time VHV is either incremented or decremented. The timeout is incremented each time that the control loop detects that the VLOW signal has not been asserted. When the timeout reaches 20 (i.e. 20 seconds) VHV is decremented. If the skin resistance has not changed the VLOW signal is asserted and the software increments VHV back to the necessary level the next time through the loop. Otherwise, VHV stays at the new voltage setting until the next timeout or the VLOW signal is asserted.

Finally the dose control sequence schedules the Dosing Mode self-tests that occur with periods greater than 1 second. These tests are the ADC and Reference Voltage test, Oscillator Accuracy Test, Battery Voltage Test and Software Timer Integrity Test. If any of these self-tests fail the software completes the dose termination sequence and transitions to EOL.

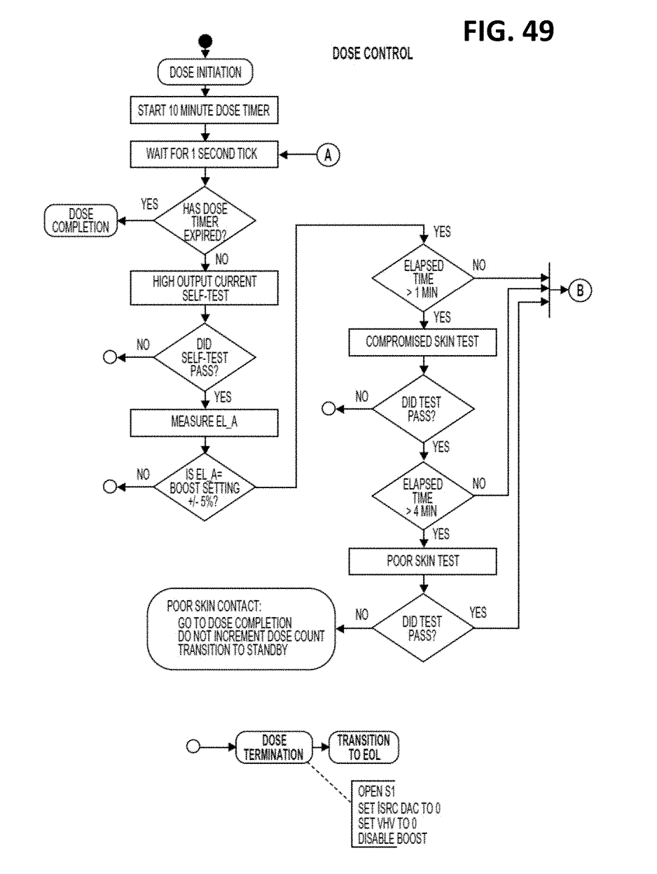

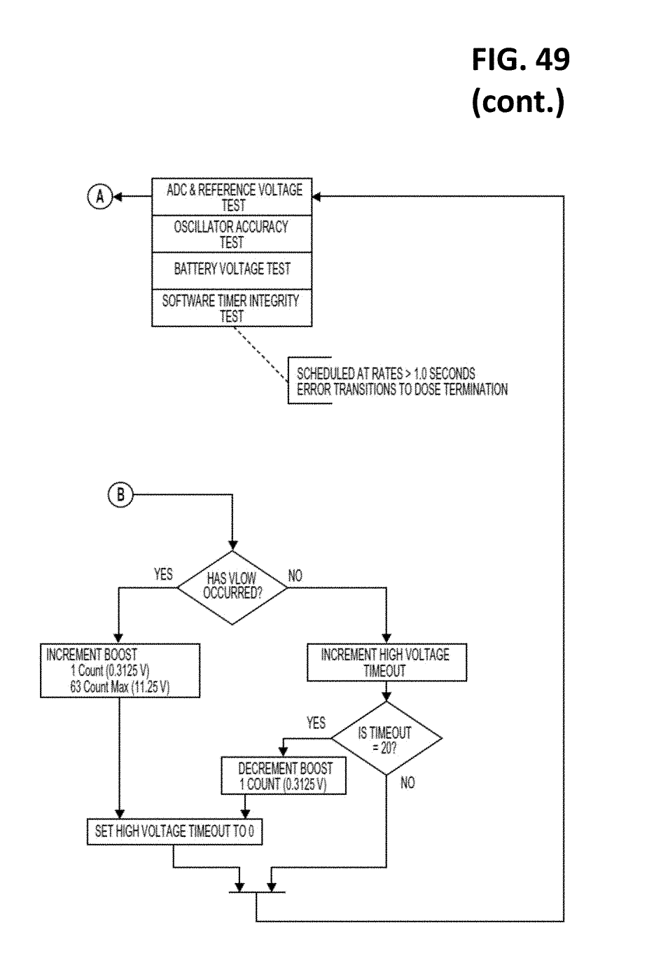

If an error other than Poor Skin Contact is encountered during the control loop, the software completes the dose termination sequence and transitions to End of Life. If Poor Skin Contact is detected, the software starts the dose completion sub-flow, but does not increment the dose count. The dose control loop is exited under normal conditions once the 10 minute dose time has elapsed. FIG. 49 shows the flow for dose control.

### Dose Completion Sequence

The dose completion sequence is started on successful delivery of a dose or when a Poor Skin Contact is detected. First the software opens S1 and sets the current source DAC to 0 counts. The register write is read back and verified. Next the software conducts the Output Current Off self-test to verify that current is not above the leakage threshold. The software sets VHV to 0 V and verifies the register write by reading it back. The software verifies that VHV is off by measuring VHV and verifying that it less than 4.0 V; the expected value is Vbat. Next, the software disables the boost circuit and verifies the register write. The anode voltage is measured to verify that the potential is low. Next, the Anode/Cathode voltage difference test (the off-current test) is completed.

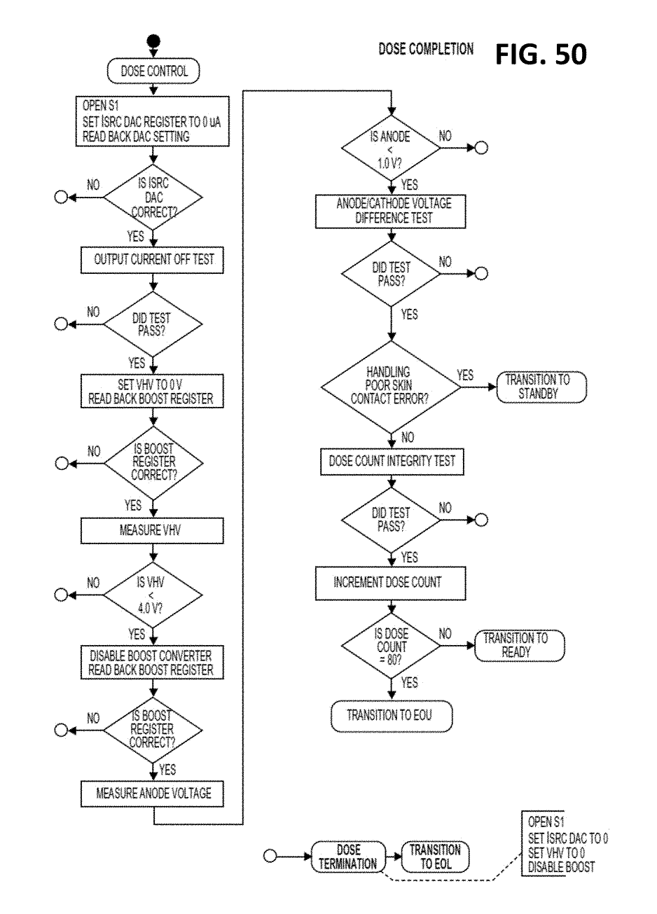

If the software is handling Poor Skin Contact detection, it exits the dose completion sequence and transitions to Standby. Otherwise, the software performs the dose count integrity test, if the test passes the dose count is incremented and the LCD is updated. If the dose count is 80, the software transitions to End of Use, otherwise the software transitions to Ready. If the software detects an error in the dose completion sequence, the dose termination sequence is completed and the software transitions to End of Life. FIG. 50 shows one example of a flow diagram for dose completion.

### Standby State

The Standby state is used to indicate that poor skin contacted was detected during the Dosing state. On entry to the state, the software logs a standby record with timestamp to NVM. While in Standby state, the output current is disabled, self-tests are suspended, and the software flashes the red LED twice a second and plays a sequence of long and short tones on the beeper. After 15 seconds, the software transitions to the Ready state.

The 250-ms task is used to produce the flashing sequence of the red LED and the tones played on the beeper. This task is also used to detect when 15 seconds have passed and initiates the transition to the next state.

The one-second task is used to service the watchdog.

### End of Use State

The software enters the End of Use state when the device has reached its 80 dose limit or its time limit of 24 hours. On entry to the state, the software logs the finish code, timestamp, and battery voltage to NVM. While in End of Use state, the output current is disabled, the final dose count is displayed on the LCD, and the red LED flashes. The software monitors the button for a press and hold event, and periodically executes self-tests.

The 50-ms task is used to detect button presses, using the functions provided by the Button driver. If the software detects a button press and hold for 6 seconds, a transition to Shutdown state is initiated.

The 250-ms task is used to produce the flashing sequence of the red LED.

The one-second task is used to schedule and execute self-tests, and service the watchdog. This task is also used to run the Battery Voltage Test once every 10 minutes. If the battery is below the low voltage threshold, the software initiates a transition to the End of Life State.

### End of Life State

On entry to the End of Life state, the software logs the reason for transition, the timestamp, and the battery voltage to NVM. The device may enter the End of Life (EOL) state when forced by errors (including failing a self-test such as the off-current test). While in End of Life state, the output current is disabled, the red LED flashes and the beeper sounds a sequence of short tones. The software monitors the button for a press and hold event, and periodically checks the battery level every 10 minutes.

The 50-ms task is used to detect button presses, using the functions provided by the Button driver. If the software detects a button press and hold for 6 seconds, a transition to Shutdown state is initiated.

The 250-ms task is used to produce the flashing sequence of the red LED and produce the short tones on the beeper.

The one-second task is used to schedule and execute self-tests, and service the watchdog. This task is also used to run the Battery Voltage Test once every 10 minutes. If the battery is below the depleted threshold, the software initiates a transition to the Shutdown State.

### Shutdown State

The Shutdown state is the final state of the device. On entry to the state, the software logs the reason for transition, the timestamp, and the battery voltage to NVM and disables the LEDs, the LCD, and the beeper.

While in the Shutdown state, the output current is disabled. The software does nothing but service the watchdog using the one-second task. The software does not exit this state.

### Self-Tests

As discussed above, the system or device may include a set of self-tests to monitor the device operating parameters to detect faults in device hardware or software, or in usage conditions. The off-current module may be one form of a self-test. The self-test may derive from requirements, risk and reliability analysis activities. The tolerance ranges specified for test limits derive included herein (including the thresholds such as the Off-Current Threshold) are exemplary only. These example tolerances may depend upon tolerances of hardware components. Software, hardware and firmware (including logic/algorithms) of the self-tests may check against a specific limit value that does not vary.

### Self-Test Scheduling and Sequencing

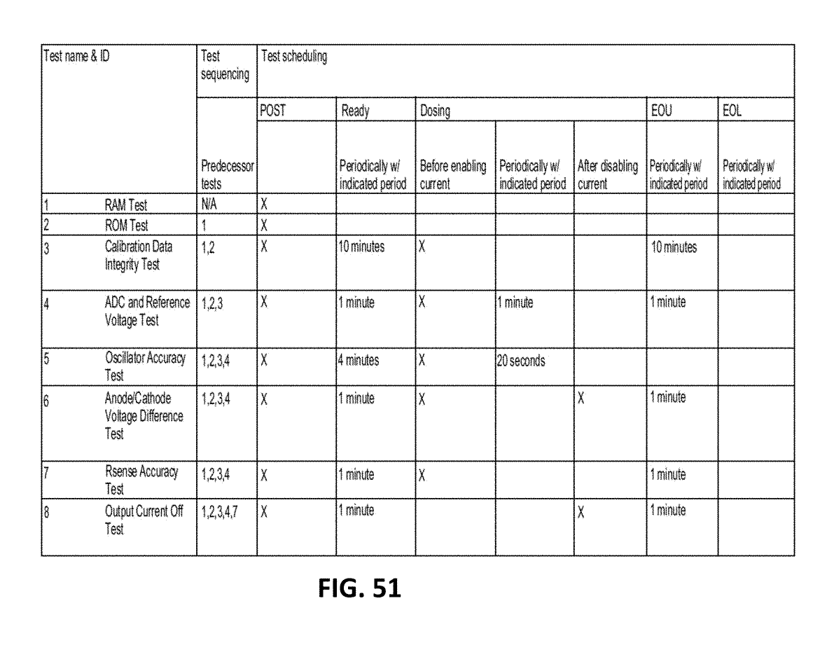

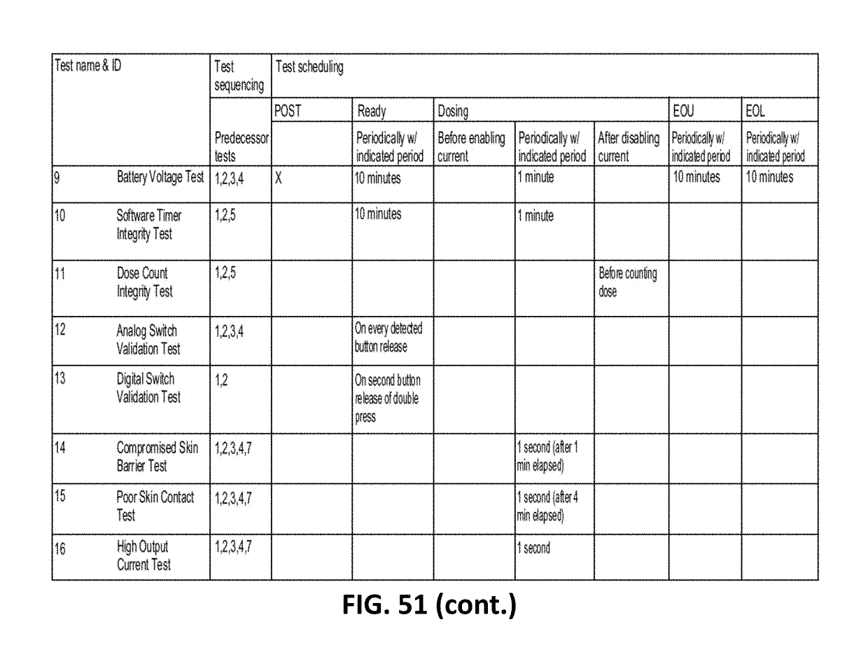

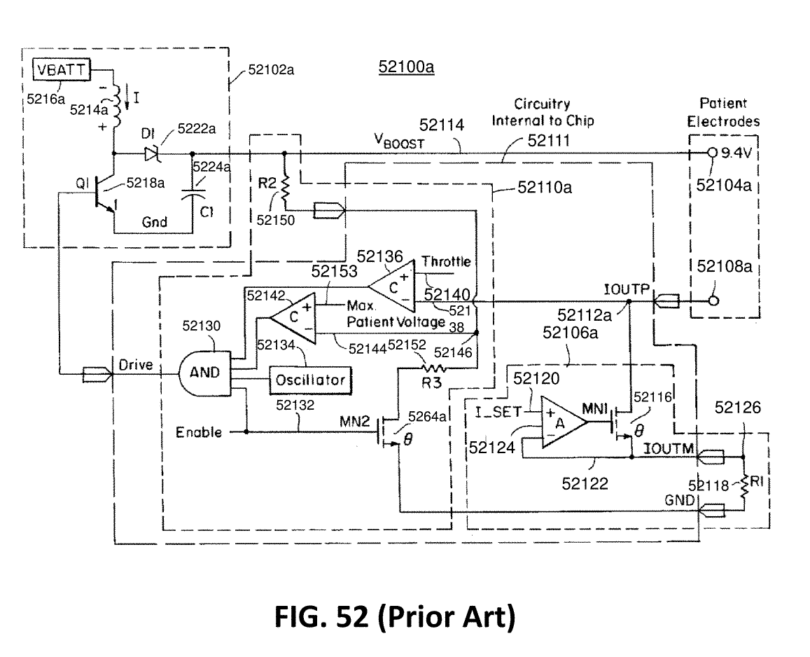

The subset of self-tests run and the scheduling of those tests may vary depending on the device's operating mode, as discussed above. FIG. 51 shows table 1, which shows self-tests that can be run in each mode and when those tests run. Standby Mode is not shown because self-tests are deferred until the return to Ready Mode. Standby lasts only 15 seconds, and with the most frequent tests running only once a minute in non-dosing modes, Standby mode would be exited before any tests would run.

The test scheduling indicated in FIG. 51 is in some cases more frequent than would be suggested by the detection times stated in the requirements. This allows for an implementation that requires several consecutive failures before a fault is set in cases where there may be significant variability of measured results from test to test. In the case of the Oscillator Accuracy Test, this allows fault detection within the required real time stated in the requirements, even if the oscillator is operating at the extreme low limit, just above the point of a hardware reset.

In many cases the correct execution of a particular test depends on the correct operation of other hardware, firmware and/or software elements that are checked by other tests. This may help determine an order in which tests must run for valid results. Predecessor tests are those that must pass before the result of a given test can be considered valid. For example, the ADC and Reference Voltage Test must pass before any test using the ADC runs.

One special case is the ROM Test. Because all code, including that for the ROM Test, is stored in ROM, it's not possible to pass the ROM Test prior to using ROM.

RAM Test

The RAM Test verifies that each address in RAM can be read and written to. The test is performed in assembly language startup code, before RAM and stack initialization or C startup. The values 0x55 and 0xAA are written to and then read from each byte of RAM to verify every bit is functioning. The test first writes 0x55 to each byte of RAM. Then it reads each byte, compares it to 0x55, and writes 0xAA to the byte. Finally, it reads each byte of RAM and compares the values to 0xAA. If any of the comparisons fail, the test fails. Otherwise, the test passes.

### ROM Test

The ROM Test verifies the contents of ROM. The test calculates an 8-bit checksum of ROM, which is a summation of all the values in ROM. At manufacture the last byte of ROM will be set so that the checksum will equal 0xFF. When the test is run, it calculates the checksum for the ROM and compares it to 0xFF. If the checksum is not equal to 0xFF, the test fails. Otherwise, the test passes.

### Calibration Data Integrity Test

The Calibration Data Integrity Test verifies the contents of calibration data stored in the internal EEPROM. These data include the boot flag, the oscillator limit values, the calibrated current source DAC setting, the Rsense reading when pulled-up, and trimming values for the ADC and oscillator. These values are encoded with error detection and correction codes. The first time the calibration data integrity check runs, it decodes all calibration values via the EEPROM driver and fails if the EEPROM driver detects uncorrectable data corruption in any of the values.

After being validated by a successful first integrity test, the ADC calibration values are stored in RAM to improve the performance of the ADC driver. On subsequent integrity checks of these values, the test compares the values stored in RAM with the values stored in EEPROM. This reduces processing time by avoiding the overhead of decoding error codes. The test passes if the values in RAM and EEPROM match and fails otherwise.

For all calibration data other than the ADC calibration, subsequent integrity tests behave the same as the first. Error codes are decoded for all values, and any uncorrectable corruption results in a test failure.

### Oscillator Accuracy Test

The Oscillator Accuracy Test verifies the accuracy of the oscillator frequency using the frequency-to-voltage conversion channel of the ADC. During manufacturing, the oscillator is calibrated to 2.048 MHz±1%, and frequency-to-voltage readings at high and low limits are stored in non-volatile memory. The stored limits are between +3% and +5% on the high side, and −3% and −5% on the low side. The tolerance on the frequency-to-voltage converter is ±5%. The stack-up of these tolerances may result in the detection threshold being close to but not more than 10% from nominal, which is within the required ±10% limits of the Oscillator Accuracy Test.

When the Oscillator Accuracy Test runs, the 12-bit ADC frequency-to-voltage reading is compared to the two 12-bit limit values stored in non-volatile memory. If the ADC reading is not within limits, the test fails. Otherwise, the test passes.

In order to detect an oscillator error within in the required real time in the case where the oscillator is running slow, the test will run more frequently than it would if the oscillator were running at a nominal frequency. Reset occurs at 0.8 MHz. This is a divider of 2.5 on the nominal value of 2.048 MHz, and the same divider must be applied to the test scheduling period. For example, in order to assure detection of a low-limit oscillator within 10 minutes, the test must run every 4 minutes.

### ADC and Reference Voltage Test

The ADC and Reference Voltage Test verifies the correct operation of the ADC, the ADC multiplexer, and the relative levels of the ADC reference voltage and the Main reference voltage. The test measures the Main reference voltage using the ADC and compares it to 1 volt. In order for the test to pass, the ADC, the ADC multiplexer, the Main voltage reference and the ADC voltage reference must all be functioning correctly. If the test fails, the component that is failing cannot be determined. The test fails if the Main reference voltage is greater than 1.1 volts or less than 0.9 volts. Otherwise the test passes.

### Software Timer Integrity Test

The Software Timer Integrity Test verifies the rate of the primary software timers using a secondary software timer. The secondary software timer is given a countdown length and the current value of one of the primary timers. During Ready mode, the secondary timer initiates a check of the primary system time every ten minutes. During Dosing mode, the secondary timer initiates a check of the primary doing timer every minute. After counting down for the specified length of time, the secondary timer compares the current primary timer value to the initial value. If the values differ by more than 10% the test fails. Otherwise, the test passes.

### Dose Count Integrity Test

The dose count integrity test verifies that the dose count value in RAM has not become corrupted. A redundant copy of dose count is stored in the internal EEPROM and initialized to zero. The test is run on successful dose competition. Before incrementing the dose count, the current value stored in RAM is compared against the copy in EEPROM. If the two values match, they are both incremented and the EEPROM value is committed. If the two values do not match the test fails.

### Rsense Accuracy Test

The Rsense Accuracy Test verifies the accuracy of the Rsense resistance value. The Rsense resistor has a tolerance of 1%. During manufacturing, the Rsense pull-up is enabled and the voltage at Rsense is measured with the ADC. The 12-bit ADC value is written to the RSENSE location in NVM. This test duplicates that manufacture measurement. The Rsense pull-up is enabled and the ADC is used to measure the Rsense voltage. The measurement is compared to the one stored in NVM. The test fails if the two values differ by more than 5%. Otherwise, the test passes.

### Battery Voltage Test

The Battery Voltage Test returns the state of the battery relative to several threshold values. The test measures the battery voltage using the ADC and compares it to the battery thresholds. The test reports the battery is good if the voltage measurement is greater than 2.7 volts+/−5%. The test reports the battery is low if the voltage measurement is less than 2.7 volts+/−5% and greater than 2.3 volts+/−5%. The test reports the battery is depleted if the voltage measurement is less than 2.3 volts+/−5%.

### Analog Switch Validation Test

The Analog Switch Validation Test measures the voltage levels on both the high and low sides of the dose button switch in order to detect potential problems that could lead to erroneous switch readings. Under normal conditions with the switch open, voltage on the high side of the switch will be slightly less than battery voltage after accounting for the small voltage drop caused by the electronic components connected to the switch circuit. Under normal conditions, the voltage on the low side of the switch will be very close to ground.

Some conditions, such as contamination or corrosion, can cause the high-side voltage to drop or the low-side voltage to rise. If the high-side voltage falls to less than (0.8×battery voltage), or the low-side voltage rises to greater than (0.2×battery voltage), the switch input is in a range of indeterminate digital logic level with respect to the digital switch input. A switch voltage in this range could result in erroneous switch readings, which could manifest as false button transitions that were not initiated by the user. The Analog Switch Validation Test detects the condition before the switch voltage levels reach the point where erroneous readings could occur.

The Analog Switch Validation Test must run when the switch is in its normally-open condition so that the high- and low-side voltages can both be measured. Any change in the switch state while the test is running could cause the test to falsely fail due to measurement of the high-side voltage while the switch is closed. The user may press or release the button at any time, but there are mechanical and human limits on the minimum time between presses. Therefore, the point where the switch state is known to be open with the greatest certainty is immediately following a detected release of the button.

The Analog Switch Validation Test runs immediately following each detected button release. It uses the ITSIC ADC to make sequential measurements of the high-side voltage, the low-side voltage, and the battery voltage. The ADC is configured to sample for 6.25 ms for each measurement. If the voltage on the high side of the switch is less than or equal to (0.8×battery voltage), or if the voltage on the low side is greater than or equal to (0.2×battery voltage), the test fails.

### Digital Switch Validation Test

The Digital Switch Validation Test is similar in purpose to the Analog Switch Validation Test, but it may be simpler, faster, and coarser in its measurements.

The test uses secondary digital inputs, connected to each side of the dose button switch, to confirm the digital logic levels while the switch is open (button not depressed). The secondary digital inputs are of the same type as the primary digital inputs, and the corresponding values are expected to match.

The Digital Switch Validation Test runs after the Analog Switch Validation Test of the second button release of a double-press that meets the criteria for a dose initiation sequence. If the secondary digital input on the high side of the switch is low, or if the secondary digital input on the low side of the switch is high, the test fails.

### Output Current Off Test

In some variations, the off-current module may be configured to perform an Output Current Off Test. The Output Current Off Test may verify that the leakage current is less than some threshold (e.g., 3 μA, 9 μA, etc.) when the current source is off. The test may calculate the leakage current from the measured Rsense voltage and the low-limit Rsense resistance of 3.96 kOhms.

$${I}_{leakage} = \frac{{V}_{Rsense}}{{R}_{Rsense}}$$

$${V}_{Rsense} = {I}_{leakage} * {R}_{Rsense}$$

$${V}_{Rsense} < ( 3 µA * 3.96 kOhms )$$

$${V}_{Rsense} < 12 mV$$

The test measures the Rsense voltage using the ADC while the current source is off. Thus, in some variations, if the Rsense voltage measurement is greater than some threshold (e.g., 12 mV, 36 mV, etc.) the test fails. Otherwise, the test passes.

### Anode/Cathode Voltage Difference Test

In some variations the off-current module may also be configured to perform an Anode/Cathode Voltage Difference Test. The Anode/Cathode Voltage Difference Test may verify that when S1 is open and the current source is disabled, there is little voltage difference between the anode and the cathode. This test may check for the failure case of current flow from anode to cathode resulting from any fault in the output circuit. The test measures the anode voltage and the cathode voltage using the ADC and calculates the voltage difference between the two points. The test fails if the voltage difference is greater than some threshold (e.g., 0.85 V, 2.5 V, etc.). Otherwise, the test passes.

### High Output Current Test

The High Output Current Test verifies that the dosing current is less than 187 μA. The test measures the voltage at Rsense using the ADC and uses that voltage to calculate the current.

$${I}_{dosing} = \frac{{V}_{Rsense}}{{R}_{Rsense}}$$

$${V}_{Rsense} = {I}_{dosing} * {R}_{Rsense}$$

$${V}_{Rsense} < ( 187 µA * 3.96 kOhms )$$

$${V}_{Rsense} < 741 mV$$

An Rsense resistance at the low limit of 3.96 kOhms will result in the lowest measured Rsense voltage at 187 μA. The test fails if the measured Rsense voltage is greater than 741 mV. Otherwise, the test passes.

### Poor Skin Contact Test

The Poor Skin-Contact Test verifies that the skin resistance is less than 432 kOhms+/−5%. The test measures the voltage at Rsense using the ADC and uses that voltage to calculate the skin resistance.

$${I}_{dosing} = \frac{{V}_{Anode} - {V}_{Cathode}}{{R}_{Skin}}$$

$${I}_{dosing} = \frac{9.25 V}{432 kOhms} = 21.4 µA$$

$${V}_{Rsense} = {I}_{dosing} * {R}_{Rsense}$$

$${V}_{Rsense} > 21.4 µA * 3.96 kOhms$$

$${V}_{Rsense} > 84.7 mV$$

At 432 kOhms, this example assumes that the difference between the anode and the cathode is 9.25 V. Since the Rsense has a tolerance of 1%, 3.96 kOhms is the lowest resistance it could have. The test fails if the voltage at Rsense is less than 84.7 mV. Otherwise, the test passes.

### Compromised Skin Barrier Test

The Compromised Skin Barrier Test verifies that the skin resistance is greater than 5000 Ohms+/−5%. The test measures the cathode voltage and the anode voltage using the ADC. The test uses these two measurements to calculate the skin resistance.

$${R}_{Skin} = \frac{{V}_{Anode} - {V}_{Cathode}}{{I}_{dosing}}$$

$${V}_{Anode} - {V}_{Cathode} = {I}_{dosing} * {R}_{Skin} \\ ( {V}_{Anode} - {V}_{Cathode} ) > ( 170 µA * 5000 Ohms )$$

$$( {V}_{Anode} - {V}_{Cathode} ) > 0.85 V$$

The test fails if the difference between the anode voltage and the cathode voltage is less than 0.85 V. Otherwise, the test passes.

### Drivers

The low-level hardware drivers provide functions to configure and use the corresponding system hardware. The drivers do not maintain timing information. Modules that use the drivers must manage any necessary timing. In some cases the drivers maintain state information pertaining to the hardware to which they provide an interface.

### Timer

The Timer driver uses the hardware timers in the CPU to provide a variety of timing functions, including: (a) a system tick driven by a periodic interrupt every 8 ms; (b) periodic ticks derived from the system tick and occurring every 50, 100, 250, 500, or 1000 ms; (c) a system timer that counts the number of seconds since power was applied to the system; (d) a dose timer that counts down the duration of a dose, in seconds; and (e) a button timer that counts down the time window for a button double-press for dose initiation.

The Timer driver uses hardware Timer0 as an 8-bit timer in auto-reload mode to provide the 8-ms system tick. Timer0 generates an interrupt each time it rolls over. To minimize interrupt processing time, the interrupt handler simply increments an 8-bit counter, sets a local flag indicating that the system tick occurred, and samples the button input (see Section 5.4.2 Dose Button). The driver provides a function for the main loop to check for occurrence of the tick. The 8-bit counter rolls over every 2.048 seconds. This allows generation of periodic ticks with periods up to that value.

When the main loop sees that the system tick has occurred, it calls the timer processing function, which updates software timers as appropriate. This function uses the system tick counter to decrement the dose and/or button timers once per second if they are active, and increment the system lifetime timer once per second. It also clears the system tick flag, indicating that processing is complete for that tick.

The Timer driver uses the system tick to calculate periodic ticks with periods that are multiples of the system tick. Nominally the periods available are 50, 100, 250, 500, or 1000 ms. However, not all these periods are integer multiples of 8 ms, so the exact period is less in some cases, due to truncation. The Timer driver provides functions to check for the occurrence of each periodic tick, as well as a function to synchronize all periodic ticks to the current system tick value.

### Dose Button

The Dose Button driver contains functions for sampling, debouncing, and detecting transitions on the button input.

The button input is sampled every 8 ms in the Timer driver periodic interrupt handler. This is necessary to achieve button sampling at a regular and sufficiently high rate. Execution of each iteration of the main loop spans several periodic interrupts and varies in duration with execution path.

The button is sampled into a circular buffer that holds eight samples. The six most recent samples are used by the debounce algorithm to determine the state of the button. All six samples must be the same to identify a valid button state. If the buffer contains a mix of low and high sample values, the button is determined to be in a bouncing or transition state. The Button driver keeps track of the state of the button from the previous time the debounce algorithm was applied and can thus identify transitions. A function is provided to check for a button release. It can be called approximately every 50 ms by tasks reading the button to provide acceptable user responsiveness to inputs. A release transition requires at least six samples with the button depressed, followed by at least six samples with it released. Therefore approximately 100 ms of sampling is required to identify a button press.

### LCD

The LCD driver provides the software interface for displaying a two-digit number on the LCD. The driver supports display of integers 0-99. Input values 0-9 do not display a leading zero. The driver also exposes the LCD control functions: enable, disable, and blank

The left and right digits are designated Digit 1 and Digit 2 respectively. Each of the two digits has seven segments. Segments are labeled A-F, starting with the top segment and moving clockwise; the center segment is labeled G. The ITSIC may be capable of driving up to 80 LCD segments. There are 20 segment control lines and 4 backplane lines (also called common lines) that are multiplexed to control each of the available 80 segments. In this application only 14 LCD segments are used with all 4 backplanes.

### LED

The LED driver provides the software interface for controlling green and red LEDs. Fixed current settings are used to drive the LEDs according to the device's power budget. The green LED is connected to the LED1 current source and driven at 2.5 mA. The red LED is connected to the LED2 current source and driven at 1.4 mA. The driver uses the LED_BEEP register to turn on, turn off or toggle each LED.

### Beeper

The Beeper driver provides the software interface for controlling the audio transducer. The operating frequency range is 1000-4875 Hz in 125 Hz steps.

When turning on the audio transducer, the driver configures the transducer to be driven by the voltage boost circuit. This allows for control of the audio volume by adjustment of the boost voltage. However, the driver does not set voltage boost. The application is responsible for setting the appropriate boost level before enabling the transducer. Voltage boost can be configured using the Boost Controller driver.

The driver controls the audio transducer through the LED_BEEP and BEEP_FC registers.

### Voltage Boost Controller

The Voltage Boost Controller driver provides the software interface for controlling the voltage boost block. This circuit is responsible for boosting battery voltage to the higher levels required to maintain dosing current output or drive the piezo audio transducer at sufficient volume.

The driver supports boost levels over the full operating range: 0.0 to 19.6875 volts in 0.3125 volt steps. The minimum boost voltage is determined by the battery voltage; settings below battery voltage result in output equal to battery voltage. The boost circuit charge time is configurable in hardware but set to a fixed value of 1.5 microseconds on driver initialization. Further, the driver provides functions for reading back the voltage control setting and enabling/disabling the boost circuit.

The driver provides a function to poll the boost over-voltage signal. The over-voltage signal is asserted if the voltage output exceeds 21.0 volts. The driver controls the boost circuit through the BOOST_0, BOOST_1, EOV and IT1 registers.

### Current Controller

The Current Controller driver provides the software interface for controlling the current source block. The current source output level is controlled by a 10-bit DAC. The driver allows for current output over the full operating range of the current source. The driver controls the current source through the ISRC_0, ISRC_1, EVL and IT0 registers.

The driver provides functions to enable or disable the current source, set the DAC value, read back the DAC value, and enable or disable the Rsense pull-up resistor.

The driver also provides an interface to the current control block's low voltage signal (VLOW). During initialization this signal is configured to monitor the gate voltage of M2. A function to monitor the state of the signal is provided.

### Trimming

The current controller requires trimming to achieve the desired accuracy at 170 μA. The uncalibrated current controller has an accuracy of ±5%, while the calibrated current controller has an accuracy of ±0.5%. The 10-bit DAC value to produce a current of 170 μA is determined and written to the ISRC_170 location in NVM during manufacture. This value is read from NVM and written to the ISRC registers when the current source is enabled.

### Analog to Digital Converter (ADC)

The ADC driver provides the software interface for configuring and using the ADC. The ADC has 12-bit resolution with three possible input ranges, configurable conversion time, and selectable inputs. The ADC inputs are grouped by full-scale range: low (0.0 to 2.0 volts), medium (0.0 to 3.6 volts) and high (0.0 to 24.0 volts).

The driver provides a function for configuring the input select, specifying the conversion time and starting a conversion. The conversion time range is 0.78125 to 100 ms. The start-conversion function is non-blocking and the conversion is asynchronous. Completion of the conversion is signaled by the ADC done interrupt. The driver is responsible for handling this interrupt and storing the counts. A function is provided for the application to determine if an ADC read is in progress.

The ADC driver is responsible for applying calibration gain and offsets for the appropriate input range. The calibrations are applied when the application reads the result of a completed conversion. Calibrations are stored locally to the driver and a function is provided to return a reference to the data structure. This reference is used to populate the calibration values from NVM and to conduct the Calibration Data Integrity Test.

The driver controls the ADC through the ADC_CTRL, ADC_MSB, ADC_LSB and EADC registers.

### Trimming

The output of the ADC must be trimmed to achieve the desired accuracy. The output of the ADC has a gain error of ±5% and an offset error of ±5%. After trimming, the ADC output has an accuracy of ±0.5%. The trimming calculation requires two 9-bit signed values from NVM for each of the three ADC ranges. Each gain and offset is stored as an 8-bit unsigned value in NVM and there is a 6-bit value that holds all the signed bits. Therefore, there are 7 values are written to NVM by the manufacture: ADC_GAIN_HIGH, ADC_OFFSET_HIGH, ADC_GAIN_MID, ADC_OFFSET_MID, ADC_GAIN_LOW, ADC_OFFSET_LOW, and ADC_SIGNS.

### High Range

$$ADC\_result = ADC\_out * ( 1 + \frac{ADC\_GAIN \_HIGH}{4096} ) + ADC\_OFFSET \_HIGH$$

$$ADC\_result = ( ADC\_MSB << 4 ) \vert ( ADC\_LSB >> 4 ) ;$$

$$ADC\_result += ( ( ADC\_GAIN \_HIGH * ADC\_MSB ) >> 8 ) \& 0 \times FF ;$$

$$ADC\_result += ADC\_OFFSET \_HIGH ;$$

### Medium Range

$$ADC\_result = ADC\_out * ( 1 + \frac{ADC\_GAIN \_MID}{4096} ) + ADC\_OFFSET \_MID$$

$$ADC\_result = ( ADC\_MSB << 4 ) \vert ( ADC\_LSB >> 4 ) ;$$

$$ADC\_result += ( ( ADC\_GAIN \_MID * ADC\_MSB ) >> 8 ) \& 0 \times FF ;$$

$$ADC\_result += ADC\_OFFSET \_MID ;$$

### Low Range

$$ADC\_result = ADC\_out * ( 1 + \frac{ADC\_GAIN \_LOW}{4096} ) + ADC\_OFFSET \_LOW$$

$$ADC\_result = ( ADC\_MSB << 4 ) \vert ( ADC\_LSB >> 4 ) ;$$

$$ADC\_result += ( ( ADC\_GAIN \_LOW * ADC\_MSB ) >> 8 ) \& 0 \times FF ;$$

$$ADC\_result += ADC\_OFFSET \_LOW ;$$

### Watchdog

The Watchdog driver provides the software interface for initializing and servicing the watchdog. The watchdog timeout is configured to 6.144 seconds by the initialization function. If the watchdog is not serviced within this period, the watchdog hardware resets the processor. The watchdog timer is started on driver initialization. The application is responsible for servicing the watchdog.

### ITSIC Core

The ITSIC Core driver provides the software interface for controller general functions related to the ITSIC. These functions include: enabling and disabling all interrupts via the general enable bit, and reading and writing the oscillator calibration value. The driver uses the EA and OSC_CAL registers. Upon initializing the driver the oscillator calibration is set to 0.0% with interrupts disabled.

### Oscillator Calibration

The oscillator requires calibration to achieve ±1% accuracy in the 2.048 MHz system clock. The uncalibrated oscillator has an accuracy of ±30%. The 8-bit calibration value adjusts the frequency of the oscillator and is determined and written to the OSC_CAL_VALUE location in NVM during manufacture. The OSC_CAL_VALUE is read from NVM and written directly to the OSC_CAL register. After writing to the register, the oscillator requires a settling time of 1 ms.

### Internal EEPROM

The ITSIC non-volatile memory is used by firmware for two purposes: persistent data storage and redundant storage of critical run-time data. The persistent storage includes device trimming data and the usage log. The redundant storage includes run-time data that has been identified as critical to safety through risk analysis. The firmware is designed to read from and write from non-volatile memory.

The ITSIC non-volatile memory is an 8k on-chip EEPROM organized as a 128×64 bit array. EEPROM access is always an entire page (64 bits wide). The EEPROM is memory mapped and referenced from code via external data addressing. External data addresses are declared in code using the C51 xdata keyword.

To improve reliability thus lowering the effective error rate, the firmware applies error detection and correction mechanisms over the EEPROM. Three mechanisms are used, each with different integrity properties. Hamming codes are used to encode entire pages. Hamming codes are used to encode specific data fields when entire page coding is not needed. Finally parity bits are used to check integrity over data that are not used by the device during operation. The two Hamming codes are capable of correcting all 1-bit errors and detecting all 2-bit errors. Parity bits are capable of detecting any odd number of bit errors.

The software interface to the EEPROM may influence the design of the system, particularly data locality. Read access is transparent to the firmware. The core reads the entire page into a 64 bit shadow register. If the requested page is already loaded, the EEPROM is not read at all. Write access requires the firmware to control the page commit timing. A write access first reads the corresponding EEPROM page into the shadow register. When the firmware is ready to commit the page, it asserts a page clear for 1 ms, a page write for 1 ms and then resets both the page write and clear bits.

### Driver Structure

The EEPROM driver encapsulates access to the EEPROM by providing functions to read from and write to the EEPROM. The driver provides functions to decode and read data from the EEPROM. These functions provide access to the device's calibration values, boot parameters and the device ID field.

The driver also provides function to validate the integrity of these values after device initialization. The validation functions compare the value stored in RAM with the value stored in EEPROM to ensure that the copy in RAM has not become corrupted.

Finally, the driver provides functions write to the EEPROM. These functions include usage logging and updating the device power-on-code. As needed the driver may handle Hamming encode and decode operations as well as calculating parity bits on write-only fields.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

One method for transdermal delivery of active agents involves the use of electrical current to actively transport the active agent into the body through intact skin by electrotransport. Electrotransport techniques may include iontophoresis, electroosmosis, and electroporation. Electrotransport devices, such as iontophoretic devices are known in the art. One electrode, which may be referred to as the active or donor electrode, is the electrode from which the active agent is delivered into the body. The other electrode, which may be referred to as the counter or return electrode, serves to close the electrical circuit through the body. In conjunction with the patient's body tissue, e.g., skin, the circuit is completed by connection of the electrodes to a source of electrical energy, and usually to circuitry capable of controlling the current passing through the device when the device is “on” delivering current. If the substance to be driven into the body is ionic and is positively charged, then the positive electrode (the anode) will be the active electrode and the negative electrode (the cathode) will serve as the counter electrode. If the ionic substance to be delivered is negatively charged, then the cathodic electrode will be the active electrode and the anodic electrode will be the counter electrode.

A switch-operated therapeutic agent delivery device can provide single or multiple doses of a therapeutic agent to a patient by activating a switch. Upon activation, such a device delivers a therapeutic agent to a patient. A patient-controlled device offers the patient the ability to self-administer a therapeutic agent as the need arises. For example, the therapeutic agent can be an analgesic agent that a patient can administer whenever sufficient pain is felt.

As described in greater detail below, any appropriate drug (or drugs) may be delivered by the devices described herein. For example, the drug may be an analgesic such as fentanyl (e.g., fentanyl HCL) or sufentanil.

In some variations, the different parts of the electrotransport system are stored separately and connected together for use. For example, examples of electrotransport devices having parts being connected together before use include those described in U.S. Pat. No. 5,320,597 (Sage, Jr. et al); U.S. Pat. No. 4,731,926 (Sibalis), U.S. Pat. No. 5,358,483 (Sibalis), U.S. Pat. No. 5,135,479 (Sibalis et al.), UK Patent Publication GB2239803 (Devane et al), U.S. Pat. No. 5,919,155 (Lattin et al.), U.S. Pat. No. 5,445,609 (Lattin et al.), U.S. Pat. No. 5,603,693 (Frenkel et al.), WO1996036394 (Lattin et al.), and U.S. Pat. No. 2008/0234628 A1 (Dent et al.).

In general, the systems and devices described herein include an anode and cathode for the electrotransport of a drug or drugs into the patient (e.g., through the skin or other membrane) and a controller for controlling the delivery (e.g., turning the delivery on or off); all of the variations described herein may also include an off-current module for monitoring the anode and cathode when the device is off (but still powered) to determine if there is a potential and/or current (above a threshold value) between the anode and cathode when the controller for device has otherwise turned the device “off” so that it should not be delivering drug to the patient. The controller may include an activation controller (e.g., an activation module or activation circuitry) for regulating the when the device is on, applying current/voltage between the anode and cathode and thereby delivering drug.

Throughout this specification, unless otherwise indicated, singular forms “a”, “an” and “the” are intended to include plural referents. Thus, for example, reference to “a polymer” includes a single polymer as well as a mixture of two or more different polymers, “a contact” may refer to plural contacts, “a post” may indicate plural posts, etc.

As used herein, the term “user” indicates anyone who uses the device, whether a healthcare professional, a patient, or other individual, with the aim of delivering a therapeutic agent to a patient.

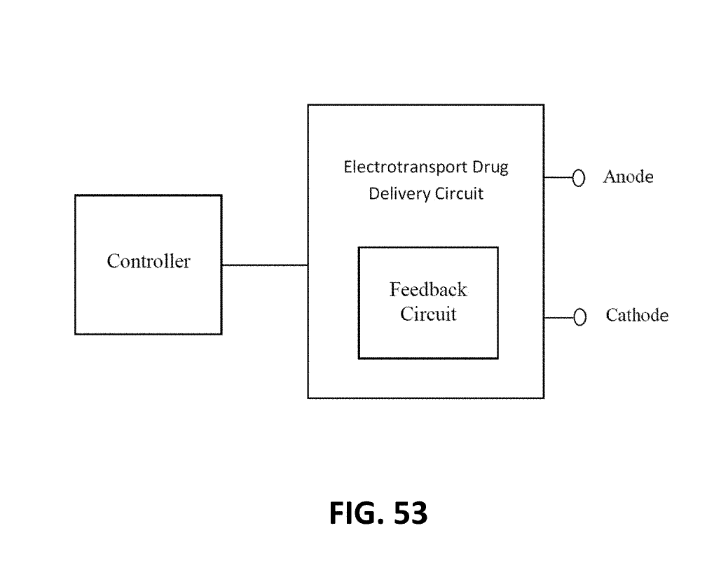

In general, the devices described herein may include control logic and/or circuitry for regulating the application of current by the device. For example, FIG. 53 illustrates a schematic for controlling the application of current to deliver drug. A feedback circuit may be controlled or regulated by a controller and be part of (or separate from) the drug delivery circuit. The controller and circuit may include hardware, software, firmware, or some combination thereof (including control logic). For example, as illustrated in FIG. 53, a system may include an anode, cathode and feedback circuit. The feedback circuit may form part (or be used by) the drug delivery module to provide current between the anode and cathode and deliver drug. The device may also include a controller controlling operation of the device. The controller may include a processor or ASIC.

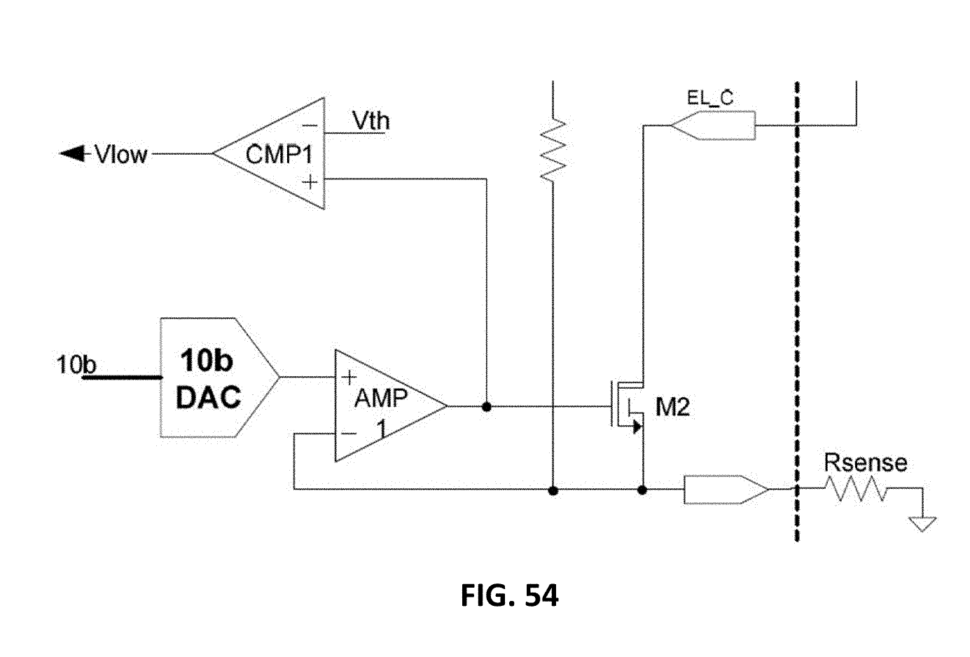

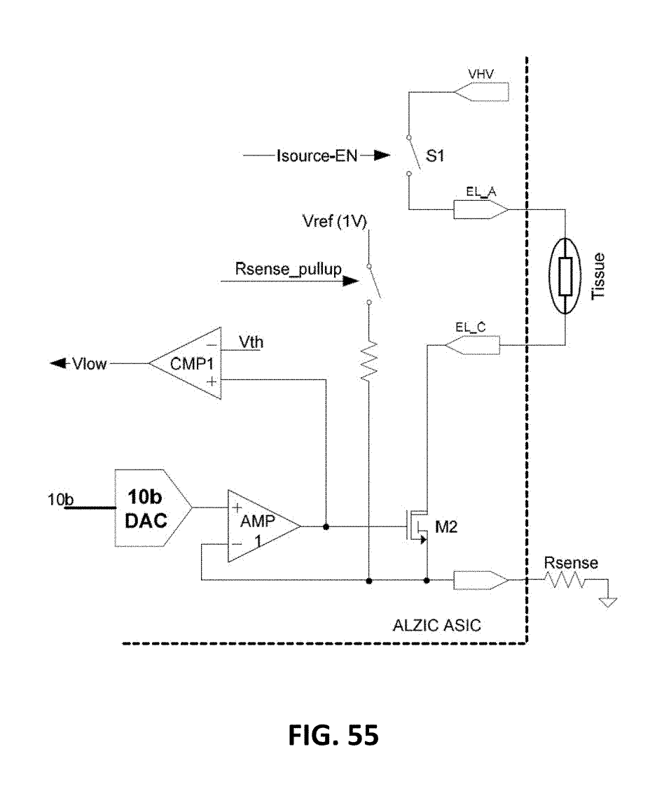

In general, the feedback circuit may be referred to as a type of self-test that is performed by the device. FIG. 54 illustrates one variation of a feedback circuit for controlling the current and/or voltage applied across the patient electrodes (anode and cathode), and is included and described in greater detail below in the context of FIG. 55.

FIG. 55 illustrates one variation of a diagram illustrating the circuit controlling the application of current to deliver drug to a patient. In this example, the drug dose is regulated by the control of the current through the electrodes (anode to cathode). The current in this example is programmable (e.g., using a 10 bit DAC), but may be preset. For example, the target current may be preset to deliver a dose using a current of 170 μA, as illustrated.

In FIG. 55, the dotted line schematically indicates ASIC components; this integrated circuit may be separate from the Rsense resistor. The patient tissue (“tissue”) completes the circuit between the anode and cathode elements. In some variations Rsense is on the printed circuit board. The Rsense may be on the circuit board (e.g., but not within the ASICS). For example, the Rsense and everything within the dotted box may be on the printed circuit board. The anode and the cathode may be connected to the patient.

In FIG. 55, above the anode is a VHV, which is the voltage source that applies the voltage to deliver a current and therefore drive delivery of drug. In some variations a Vboost may also be included as part of (or in connection with) the VHV. In addition, a switch, S1, may be included as a software-enabled switch to control the application of voltage to the anode. The S1 switch may act as a safety feature to control (via software) when current is not delivered. The voltage may be turned off completely and the switch opened so that even if there were some other voltage present, the anode would be floating. Thus, current could not be pulled through the anode to cathode because the anode is floating (and there is no source of electrons to pull current through, since it is a completely open circuit).

In this example, when the Slswitch is closed, current may then flow from the anode, through the tissue, and return through the cathode to the M2 switch (transistor). In this example, the M2 switch is a transistor (e.g., a field effect transistor) that acts as a valve to control the flow of current. The M2 switch may be referred to as a current control valve or throttle that regulates the current flow down to Rsense resistor, where it goes to the ground. Schematically, the current is throttled by the M2 switch, which may allow control of the current at various levels. For example, in FIG. 55, the current level is set to be approximately 170 uA. In this example, Rsense may be used to set the current range, and/or the maximum value. A square wave of current may be delivered.

Further, M2 may be regulated by an amplifier (e.g., Amp1). In this example, Amp1 is an analogue amplifier; the input to Amp1 is a digital to analogue converter (DAC), which is set with the target 170 μA level. Thus, a microcontroller may be used to set a digital signal using an analogue to digital converter, corresponding to the target delivery current (e.g., 170 μA).

Thus, in operation, the controller (e.g., microcontroller) may be configured so that when no current is to be delivered the DAC may be set to 0 and when current is to be delivered, it may be set to 170 uA, providing an analogue output to AMP1 that allows current to flow to Rsense. Thus, the input to M2 (gate) may be used to monitor the voltage at the transistor gate using a comparator, e.g., CMP1. In some variations the voltage at the gate is compared to a threshold (Vthreshold). If the voltage at the gate of M2 is low, it may be increased, and if it is high, it may be decreased. This feedback may be used to adjust VHV, as illustrated in FIG. 55.

Because the cathode is connected only to the current control transistor and not directly connected to the sensing circuit, potential faults in the sensing circuit are isolated from the second patient contact and could not result in additional current flow from anode to cathode, and therefore could not result in additional drug delivered to the patient.

The voltage may be changed to set the current with that DAC and AMP1. For example, the current may be set to 170 uA, and the control system described herein prevents it from going over 170 uA, providing a constant current source. That DAC, AMP1 and M2 limit the maximum amount of current that can flow through M2. Setting the DAC to 170 uA prevents the current from exceeding 170 uA regardless of the voltage. In this configuration, if the voltage is higher than it has to be, then by Ohms law, V=IR, where R is the skin resistance and I is the target 170 uA, the voltage can be limited. The M2 throttles the maximum amount of current to limit it to 170 uA, allowing the voltage to be adjusted. Since the power equals the current times the voltage, when the current is fixed (e.g., to 170 μA) the amount of power can be minimized by providing only the minimum amount of voltage. This may help conserve the batter power by using just use the minimum amount of voltage required. In practice the control and monitoring circuit may do this by adjusting the voltage to automatically drop the voltage as necessary. Monitoring the gate at M2 to maintain saturation so that the source voltage VHV value is kept above the level sufficient to deliver current at the set (e.g., 170 μA) value. Below the saturation level, the gate may deliver less than 170 uA. To prevent this, the voltage is allowed to drop until it reaches a limit at which it is saturated; and as soon as this threshold is reached, the comparator may sense that the saturation and may adjust the voltage back up. This feedback (voltage feedback) takes place at the level of the M2 gate (throttle) and provides a constant feedback loop where it is constantly comparing the gate of the M2 to a threshold value.

Because this feedback loop occurs at the throttle, e.g., rather than the cathode (by, for example, monitoring the voltage at the cathode), additional benefits may be realized. Monitoring the voltage at that gate (M2) to control the VHV allows control of the boost voltage without altering (e.g., touching) the cathode at all, e.g., maintaining the monitoring and control aspects of the system in electrical isolation. This allows separation of the control aspect from a risk management aspect of the device, preventing the device from applying inappropriate current and thereby drug. In operation, self-checks measuring the anode-cathode voltage may be performed independently of the control of the voltage and/or current across the anode and cathode, since the cathode (and/or anode) is not used for monitoring. Instead, the cathode is used to deliver the drug.

This configuration allows control of the voltage to reduce power consumption, and/or monitoring and controlling the voltage without having to monitor at the cathode, resulting in an efficiency of the system by monitoring at the throttle point where the system can meet safety objectives while only making measurements at the anode and cathode that are related to current flow through the anode cathode. Thus, the cathode does not require connection of a measurement line to the electrode (e.g., cathode). Control of the voltage is therefore independent of safety features such as error detection (e.g., leak current detection). Further, this architecture separates the voltage control mechanism from such an error detection mechanism. Error detection mechanisms may include (e.g., within the ASICS) an analogue to digital convertor and the analogue to digital convertor multiplexed to measure the voltage at the anode, the cathode, the VHV voltage, or the like. However, the feedback detection and control of the voltage and current may be regulated from the cathode (e.g., gate M2) rather than the level of the cathode.

The advantage of this logical separation may include making only measurements on that anode and cathode that are related to whether or not there is leakage current present (whether or not there is a safety problem). Measurements at the gate M2 may be constantly ongoing (e.g., every couple of clock ticks); it is not necessary to measure at the cathode in this configuration, so that the cathode remains isolated from the feedback circuit through the gate M2. The anode cathode measurement is independent verification that there is no current flowing there. By configuring the system in this manner, the cathode is isolated from the feedback that is controlling the voltage. Separation permits the feedback mechanism to be separate from the actual patient connection delivering the current. Thus, the voltage is less critical for patient safety and the monitoring and controlling of the voltage is configured to provide efficiency of the system and the battery power. Current may flow through the drain to the source of the transistor without requiring additional circuitry between the cathode and ground, reducing the chance for malfunction (e.g., additional current flow) through this additional current path. Patient safety can be dramatically affected by even small errors in the circuitry. Thus, in some variations, the system is limited so that the only connections to the anode/cathode are those that must be there, as shown and described herein.

In some variations, the systems and methods described herein use a gate (e.g., transistor M2) to isolate the patient connections, e.g., anode and cathode, from the feedback module used to control the applied voltage and to regulate the current between the patient connections. In this example, the feedback module is configured as a circuit including a comparator that compares the voltage on the transistor to a threshold voltage.

In some variations this circuit regulates the current between the patient connections so that it rides at or below the target current level (e.g., 170 μA). The circuit may sense when the current is above 170 uA.

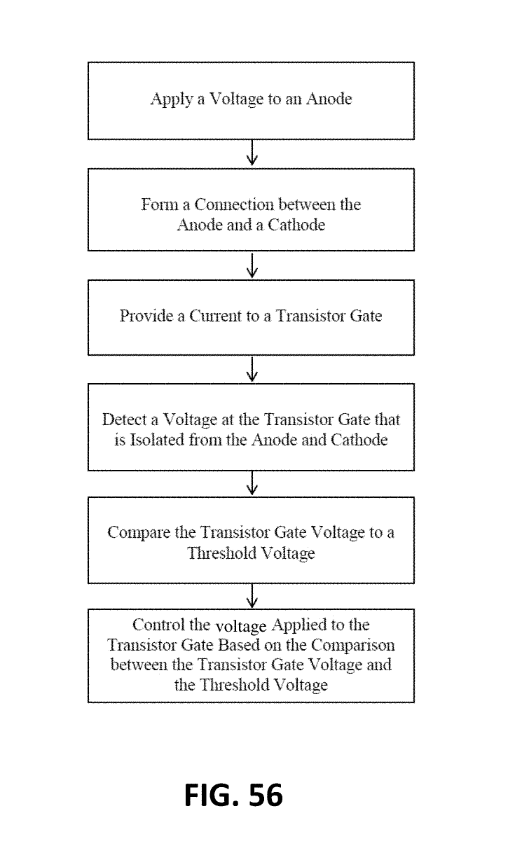

FIG. 56 schematically illustrates one variation of a method for regulating the voltage and/or current across the patient connections (e.g., anode and cathode) of an electrotransport drug delivery system. In this example, a pair of patient connections are configured to contact a patient tissue (e.g., skin) to complete the patient circuit. The first patient connection (in some configurations the anode, in other configurations, the cathode) is connected to a driving voltage source. The connection between the driving voltage source and the first patient connection may be regulated by a switch or gate, which may be regulated or controlled (e.g., by a microcontroller). The second patient connection (e.g., in some variations a cathode, in other variations an anode) is then connected in series to a transistor drain (or other throttle element), and a feedback module for monitoring and controlling the current and voltage applied between the first and second patient connections are isolated from the second patient connection by this transistor gate.

In operation, a voltage is first applied to the first patient connection (e.g., anode) after or before a connection is made by skin contact between the first and second patient connections. Current is then provided to the transistor drain downstream of the second patient contact, and a feedback module determines the voltage at the transistor gate in isolation of the first and second patient contacts. The voltage at the transistor gate is compared to a threshold voltage and this comparison is used to adjust the applied voltage at the first patient connection. In the same example, a target current may be provided (e.g., from a microcontroller) to the transistor to regulate the current between the first and second patient connections.

The constant current supply described above may be used to regulate the dosing of the system to deliver a target current (e.g., drug delivery current) at a low voltage even with variable patient resistances. For example, the circuit shown in FIGS. 54 and 55 may be used to provide a dose of drug by delivering a predetermined 170 μA drug delivery current over a dosing period (e.g., a 10 minute dose). The circuit controlling the anode and cathode shown in FIGS. 54 and 55 includes a control block containing circuitry to connect the output of the voltage boost converter (VHV) to the anode electrode (EL_A) through the switch S1. A 10 bit DAC is used to configure the current output to a set value proportional to the desired dosing current. The DAC drives AMP1 which controls the current flowing through EL_A and EL_C by driving the gate of M2. The source of M2 determines the current flow through Rsense which causes the voltage drop that is fed back into AMP1. As the skin resistance between EL_A and EL_C varies, so does the current through Rsense, which triggers a change in the output of AMP1. AMP1 becomes saturated if there is not sufficient voltage to deliver the programmed current with the resistance between EL_A and EL_C. Driver functions are available to control and monitor various the points of this circuit.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

What is claimed is:

1. A drug delivery device adapted to validate the operation of a user-selectable activation switch to deliver a dose of drug, the device comprising:
a battery;
a switch configured to be activated by a user to deliver a dose of drug;
a controller configured validate operation of the switch, wherein the switch is user-activated to deliver a dose of a drug from a drug delivery device, the controller configured to:
monitor the switch to determine a release event;
perform a digital validation of the switch following the release event using a dose switch circuit and failing the digital validation if a secondary digital input on a high side of the switch is low or if a secondary digital input on a low side of the switch is high;
perform an analog validation of the switch if the digital validation passes and failing the analog validation if a measurement of a high side voltage is less than a first predetermined fraction of a battery voltage for the drug delivery device or if a measurement of a low side voltage is greater than a second predetermined fraction of the battery voltage; and
initiate a failure mode for the drug delivery device if the analog validation of the switch fails.

2. The device of claim 1, wherein the controller monitors the switch by sequentially sampling a switch input, storing a window of sequential samples, and comparing a plurality of more recent sequential samples to a plurality of older sequential samples within the stored window of samples to detect the release event.

3. The method of claim 1, wherein the controller monitors the switch by sequentially sampling a switch input, storing a window of sequential samples, and comparing three or more recent sequential samples to three or more older sequential samples within the stored window of samples to detect the release event.

4. The method of claim 1, wherein the controller initiates the failure mode by turning off the delivery device.

5. The method of claim 1, wherein the controller initiates the failure mode by inactivating the delivery device.

6. The method of claim 1, wherein the controller re-starts a button sampling process of the drug delivery device if the digital validation of the switch fails.

7. An electrotransport drug delivery system comprising an electrical module and a reservoir module that are combined to form a unitary, activated drug delivery system prior to use, wherein:
the electrical module comprises:
control circuitry;
an electrical output connected to the control circuitry;
two or more power-on contacts between the control circuitry and a battery; and
the battery, which is isolated from the control circuitry by the two or more power-on contacts while at least one of the two or more power-on contacts remains open, and which is connected into the control circuitry when all of the two or more power-on contacts are closed by a battery contact actuator on the reservoir module when the electrical module and the reservoir module are combined; and
the reservoir module comprises:
a pair of electrodes;
an electrical input that is separate from the electrical output until the electrical module and reservoir module are combined, wherein the electrical input connects the control circuitry to the pair of electrodes when the electrical module is combined with the reservoir module; and
two or more battery contact actuators each configured to close a corresponding power-on contacts of the two or more power-on contacts when the electrical module is combined with the reservoir module, such that the battery is connected into the control circuitry, powering the system.

8. The system of claim 7, wherein the reservoir module includes a reservoir comprising fentanyl.

9. The system of claim 7, further comprising a flexible polymeric cover over each of the two or more power-on contacts.

10. The system of claim 7 further comprising a flexible polymeric cover over each of the two or more power-on contacts, wherein the seal is configured to be deformed by the two or more battery contact actuators when the electrical module is combined with the reservoir module.

11. The system of claim 7, further comprising a water-tight seal sealing the electrical output.

12. The system of claim 7, wherein the electrical output is configured to flex while continuously applying a force on the electrical input of the reservoir module to ensure good electrical connection between the two.

13. An electrotransport drug delivery device that prevents unwanted delivery of drug while in an off state when the device is powered on, the device comprising:
an anode and a cathode;
an activation circuit configured to apply current between the anode and cathode to deliver a drug by electrotransport when the device is in an on state and not in the off state; and
wherein the device is configured to shut down when there is a current flowing between the anode and cathode that is greater than an Output Current Off threshold when the device is in an off state while powered on;
further wherein the device is configured to automatically and periodically determine if there is a current flowing between the anode and cathode when the activation circuit is in the off state while powered on.

14. The device of claim 13, wherein the device is configured to determine if there is a potential difference between the anode and the cathode when the activation circuit is in the off state while powered on.

15. The device of claim 13, further configured to determine if there is a change in capacitance between the anode and cathode when the activation circuit is in the off state while powered on.

16. The device of claim 13, further configured to determine if there is a change in inductance between the anode and cathode when the activation circuit is in the off state while powered on.

17. The device of claim 13, further comprising a sensing circuit that independently determines an anode voltage and a cathode voltage and compares the potential difference between the anode voltage and cathode voltage to a threshold value.

18. The device of claim 17, further including a switch connected between a reference voltage source and a sense resistor, the off-current module configured to close the switch periodically to determine the potential difference between the anode voltage and cathode voltage.

19. An electrotransport drug delivery system having a constant current supply, the system comprising:
a power source;
a first patient contact connected to power source;
a second patient contact connected to a current control transistor; and
a sensing circuit configured to measure voltage at the transistor, wherein the sensing circuit is configured to provide feedback controlling power at the first patient contact, wherein the second patient contact is connected to the sensing circuit only through the current control transistor so that the second patient contact is electrically isolated from the sensing circuit.

20. The system of claim 19, wherein the current control transistor is controlled by an amplifier receiving input from a microcontroller.

21. The system of claim 19, wherein the sensing circuit is configured to compare the voltage applied to the transistor to a threshold voltage.

22. The system of claim 19, wherein the sensing circuit provides input to a feedback circuit.

23. The system of claim 22, wherein the feedback circuit automatically controls the power source based on the comparison between the voltage at the transistor and the threshold voltage to maintain constant current while minimizing power consumption.
